(19) United States
(12) Patent Application Publication
(10) Pub. No.: US 2005/0089914 A1
(43) Pub. Date: Apr. 28, 2005

(54) Methods for determining and measuring risk of arteriosclerotic disease, microarray, apparatus and program for determining risk of arteriosclerotic disease

(75) Inventor: Yoshimitsu Yamasaki, Hyogo (JP)

Correspondence Address: ARMSTRONG, KRATZ, QUINTOS, HANSON & BROOKS, LLP, 1725 K STREET, NW, SUITE 1000, WASHINGTON, DC, 20006, US

(73) Assignee: OSAKA INDUSTRIAL PROMOTION ORGANIZATION, Osaka-shi (JP)

(21) Appl. No.: 10/961,043

(22) Filed: Oct. 12, 2004

Related U.S. Application Data
(63) Continuation-in-part of application No. PCT/IB03/01368, filed on Apr. 14, 2003.

(30) Foreign Application Priority Data
Apr. 12, 2002 (JP) 2002-111132

(51) Int. Cl. C12Q 1/68

(52) U.S. Cl. 435/6

(57) ABSTRACT

An object is to provide a method for determining the risk of arteriosclerotic disease which can accurately determine the tendency to develop an arteriosclerotic disease or tendency for advance thereof as the risk of arteriosclerotic disease and can be utilized in prevention and treatment of arteriosclerosis and to provide, for example, a kit for analyzing genetic polymorphism and apparatus for determining the risk of arteriosclerotic disease for use in determination of the risk. A method for determining the risk of arteriosclerotic disease includes a risk evaluation process for evaluating the risk of arteriosclerosis caused by genetic polymorphisms, based on the risk of arteriosclerosis inherent to a combination of plural genetic polymorphisms, from the genotype of a subject on the genetic polymorphisms, in which the combination of plural genetic polymorphisms includes at least one combination of genetic polymorphisms having a significant positive correlation with the carotid arterial intima-media thickness.

### CROSS-REFERENCE TO RELATED APPLICATIONS

This is a continuation-in-part of Application PCT/IB03/01368, filed on Apr. 14, 2003.

### BACKGROUND OF THE INVENTION

1. Field of the Invention

The present invention relates to determination of the risk of arteriosclerotic disease. More specifically, it relates to a method for determining the risk of arteriosclerotic disease, a method for revealing an arteriosclerotic disease-associated factor, a method for measuring the risk of arteriosclerotic disease, a method for detecting a genetic polymorphism, a genetic marker, a kit for analyzing genetic polymorphism, an array, apparatus and a program for determining the risk of arteriosclerotic disease. These can be utilized for prevention, treatment and diagnosis of arteriosclerotic disease.

2. Description of the Related Art

Environmental factors such as hypertension, diabetes, hyperlipemia, obesity and smoking relate to the onset of arteriosclerotic disease (ischemic heart disease) as risk factors. In addition, family history is one of risk factors. Molecular biological techniques have recently developed and revealed various genetic polymorphisms on genes relating to arteriosclerosis, and their relations with diseases have been studied.

If the risk of an arteriosclerotic disease such as tendency to develop the disease and tendency for the advance thereof can be determine based on information on genotypes of genetic polymorphisms of a subject by taking genetic polymorphisms involved in the disease as risk factors, a subject having a high risk can be careful of preventing the disease early and routinely and predict advance after onset, which leads to more detail diagnosis and treatment.

A single genetic polymorphism is studied in clinical relation studies which have been reported on genetic polymorphisms including SNP in arteriosclerotic diseases. Then the ratio of patients with myocardial infarction to healthy subjects is determined to calculate the odds ratio of tendency to develop myocardial infarction in a population of one genotype and in a population of another genotype on the genetic polymorphism, respectively. According to such a survey method, most of polymorphisms, however, do not show a significant difference, and risks such as tendency to develop a disease and tendency for the advance of the disease cannot be predicted from the genetic polymorphisms (Yamada Y, Izawa H, Ichihara S, Takatsu F, Ishihara H, Hirayama H, Sone T, Tanaka M, Yokota M. Prediction of the risk of myocardial infarction from polymorphisms in candidate genes. N.Engl.J.Med. 2002; 347(24):1916-23).

### SUMMARY OF THE INVENTION

An object of the present invention is to solve problems on conventional determination of the risk of arteriosclerotic disease and to achieve the following objects. Specifically, an object of the present invention is to provide a method for determining the risk of arteriosclerotic disease which can accurately determine the tendency to develop an arteriosclerotic disease or tendency for the advance of the disease as the risk of arteriosclerotic disease and can be utilized in prevention and treatment of arteriosclerotic disease. Another object of the present invention is to provide a method for revealing an arteriosclerotic disease-associated factor, a method for measuring the risk of arteriosclerotic disease, a method for detecting a genetic polymorphism, a genetic marker, a kit for analyzing genetic polymorphism, an array for determining the risk of arteriosclerotic disease, an apparatus for determining the risk of arteriosclerotic disease and a program for determining the risk of arteriosclerotic disease which are used in, for example, determination of the risk.

The present inventors have quantitatively analyzed the relation between a multiplicity of genetic polymorphisms and the carotid arterial intima-media thickness and have found that a combination of plural the genetic polymorphisms significantly affects the carotid arterial intima-media thickness additively or synergistically.

The present invention has been accomplished based on these findings of the present inventors, and the means for solving the problems are as follows.

<1> A method for determining the risk of arteriosclerotic disease, comprising a risk evaluation process for evaluating the risk of arteriosclerosis caused by genetic polymorphisms, based on the risk of arteriosclerosis inherent to a combination of plural genetic polymorphisms, from the genotype of a subject on the genetic polymorphisms,
wherein the combination of plural genetic polymorphisms includes at least one combination of plural genetic polymorphisms having a significant positive correlation with the carotid arterial intima-media thickness.

<2> A method for determining the risk of arteriosclerotic disease according to the above-mentioned <1>, wherein the risk of arteriosclerosis inherent to the combination of plural genetic polymorphisms is set in accordance with whether or not the combination has a significant positive correlation with the carotid arterial intima-media thickness.

<3> A method for determining the risk of arteriosclerotic disease according to the above-mentioned <1>, wherein the risk of arteriosclerosis inherent to the combination of plural genetic polymorphisms is set in accordance with the odds ratio where the combination has a significant positive correlation with the carotid arterial intima-media thickness.

<4> A method for determining the risk of arteriosclerotic disease according to the above-mentioned <1>, wherein the risk of arteriosclerosis inherent to the combination of plural genetic polymorphisms is set in accordance with an an amount of increase in the carotid arterial intima-media thickness.

<5> A method for determining the risk of arteriosclerotic disease according to any one of the above-mentioned <1> to <4>, wherein the combination of plural genetic polymorphisms comprises at least one set of arteriosclerosis-associated genetic polymorphisms selected from sets of arteriosclerosis-associated genetic polymorphisms shown in following Tables 9-1 and 9-2.

TABLE 9-1

| Poly- | | | Poly- | | | | | |
|---|---|---|---|---|---|---|---|---|
| morphism | | Name of Genetic | morphism | | Name of Genetic | | Chi- | Odds |
| No. | Category | Polymorphism | No. | Category | Polymorphism | Frequency | square | Ratio |
| 42 | 12 | hepatic_lipase | 61 | 1 | RAGE(Gly82Ser) | 0.016 | 11.8 | 99 |
| 41 | 1 | PPAR_gamma | 61 | 1 | RAGE(Gly82Ser) | 0.014 | 9.7 | 99 |
| 18 | 23 | E-selectin | 58 | 1 | LTA(C804A(Thr26Asn)) | 0.014 | 9.6 | 99 |
| 18 | 23 | E-selectin | 57 | 3 | LTA(A252G) | 0.013 | 9.4 | 99 |
| 61 | 1 | RAGE(Gly82Ser) | 70 | 12 | Thrombopoietin(A5713G) | 0.013 | 9 | 99 |
| 7 | 12 | TGF beta | 61 | 1 | RAGE(Gly82Ser) | 0.012 | 8.8 | 99 |
| 2 | 23 | Enos 298 | 11 | 1 | MCP-1(A-2518G) | 0.011 | 7.9 | 99 |
| 27 | 3 | GPIIbIIIa | 61 | 1 | RAGE(Gly82Ser) | 0.018 | 9.1 | 11.9 |
| 61 | 1 | RAGE(Gly82Ser) | 63 | 12 | CYP2C9_3(Leu359Ile) | 0.018 | 9 | 11.8 |
| 61 | 1 | RAGE(Gly82Ser) | 68 | 23 | IL-10(C-819T) | 0.017 | 8.3 | 11.1 |
| 10 | 12 | MMP-12 | 61 | 1 | RAGE(Gly82Ser) | 0.016 | 8.1 | 10.8 |
| 47 | 12 | serotonin_2A_receptor | 61 | 1 | RAGE(Gly82Ser) | 0.016 | 8 | 10.8 |
| 6 | 12 | interleukin6(C-634G) | 61 | 1 | RAGE(Gly82Ser) | 0.015 | 7.3 | 10 |
| 51 | 23 | FactorXII | 61 | 1 | RAGE(Gly82Ser) | 0.015 | 7.1 | 9.8 |
| 38 | 3 | ABCA1 | 20 | 1 | ACE | 0.02 | 7.6 | 6.3 |
| 20 | 1 | ACE | 52 | 3 | glycoproteinIa(C807T) | 0.02 | 7.5 | 6.3 |
| 37 | 3 | MTHFR(C677T) | 58 | 1 | LTA(C804A(Thr26Asn)) | 0.029 | 10.4 | 6 |
| 20 | 1 | ACE | 54 | 1 | GP Ia(G873A) | 0.019 | 7 | 6 |
| 37 | 3 | MTHFR(C677T) | 57 | 3 | LTA(A252G) | 0.027 | 9.2 | 5.5 |
| 1 | 23 | Enos786 | 57 | 3 | LTA(A252G) | 0.134 | 12.7 | 4.4 |
| 60 | 3 | Adiponectin(G276T) | 66 | 23 | Methionine | 0.03 | 8.3 | 4.4 |
| | | | | | synthase(A2756G(Asp919gly)) | | | |
| 36 | 12 | PAI-1 | 62 | 1 | Thrombospondin-1(A2210G) | 0.482 | 88.9 | 4.3 |
| 57 | 3 | LTA(A252G) | 69 | 3 | IL-18(G-137C) | 0.111 | 10.4 | 4.3 |
| 58 | 1 | LTA(C804A(Thr26Asn)) | 69 | 3 | IL-18(G-137C) | 0.111 | 10.4 | 4.3 |
| 7 | 12 | TGF beta | 62 | 1 | Thrombospondin-1(A2210G) | 0.395 | 76.9 | 4 |
| 49 | 1 | matrilyn promoter(A-181G) | 62 | 1 | Thrombospondin-1(A2210G) | 0.488 | 79 | 3.9 |
| 62 | 1 | Thrombospondin-1(A2210G) | 71 | 1 | LDL receptor related protein(C766T) | 0.451 | 77.9 | 3.9 |
| 21 | 1 | AT2-receptor | 62 | 1 | Thrombospondin-1(A2210G) | 0.482 | 77 | 3.8 |
| 62 | 1 | Thrombospondin-1(A2210G) | 68 | 23 | IL-10(C-819T) | 0.492 | 76.7 | 3.8 |
| 55 | 12 | bradykinin B2 | 62 | 1 | Thrombospondin-1(A2210G) | 0.381 | 68.1 | 3.8 |
| | | receptor(C-58T) | | | | | | |
| 57 | 3 | LTA(A252G) | 64 | 1 | interleukin 1 beta(C3953T) | 0.139 | 11.5 | 3.8 |

TABLE 9-2

| Poly- | | | Poly- | | | | | |
|---|---|---|---|---|---|---|---|---|
| morphism | | Name of Genetic | morphism | | | | Chi- | Odds |
| No. | Category | Polymorphism | No. | Category | Name of Genetic Polymorphism | Frequency | square | Ratio |
| 50 | 1 | p22phox | 62 | 1 | Thrombospondin-1(A2210G) | 0.481 | 73.6 | 3.7 |
| 24 | 1 | beta2 | 62 | 1 | Thrombospondin-1(A2210G) | 0.482 | 72.6 | 3.7 |
| | | Adrenoreceptor(C79T) | | | | | | |
| 53 | 1 | apolipoproteinE(E3 | 57 | 3 | LTA(A252G) | 0.14 | 10.7 | 3.7 |
| | | inexon 4(Arg 158Cys) | | | | | | |
| 51 | 23 | FactorXII | 62 | 1 | Thrombospondin-1(A2210G) | 0.499 | 72.8 | 3.6 |
| 62 | 1 | Thrombospondin-1(A2210G) | 65 | 12 | IL-18(C-607A) | 0.439 | 68.2 | 3.6 |
| 8 | 3 | TNFalfa(G-238A) | 57 | 3 | LTA(A252G) | 0.149 | 11.6 | 3.6 |
| 57 | 3 | LTA(A252G) | 59 | 23 | Thrombospondin4(G1186C(Ala387Pro)) | 0.147 | 11.4 | 3.6 |
| 1 | 23 | Enos786 | 58 | 1 | LTA(C804A(Thr26Asn)) | 0.135 | 10.6 | 3.6 |
| 68 | 23 | IL-10(C-819T) | 72 | 1 | PGC-1(G1302A(Thr394Thr)) | 0.044 | 9.9 | 3.6 |
| 29 | 23 | HPA-2(Thr145Met) | 40 | 1 | PON1(Gly192Arg) | 0.031 | 7 | 3.6 |
| 42 | 12 | hepatic_lipase | 62 | 1 | Thrombospondin-1(A2210G) | 0.425 | 67 | 3.5 |
| 17 | 12 | ICAM1(E469K) | 62 | 1 | Thrombospondin-1(A2210G) | 0.457 | 66.4 | 3.5 |
| 47 | 12 | serotonin_2A_receptor | 62 | 1 | Thrombospondin-1(A2210G) | 0.401 | 64 | 3.5 |
| 14 | 23 | CRP(G1059C) | 57 | 3 | LTA(A252G) | 0.145 | 11.2 | 3.5 |
| 41 | 1 | PPAR_gamma | 72 | 1 | PGC-1(G1302A(Thr394Thr)) | 0.043 | 9.2 | 3.5 |
| 6 | 12 | interleukin6(C-634G) | 62 | 1 | Thrombospondin-1(A2210G) | 0.493 | 64.2 | 3.4 |
| 25 | 12 | beta-adrenergic | 62 | 1 | Thrombospondin-1(A2210G) | 0.42 | 64 | 3.4 |
| | | receptor(A46G) | | | | | | |
| 57 | 3 | LTA(A252G) | 65 | 12 | IL-18(C-607A) | 0.076 | 16 | 3.4 |
| 26 | 23 | HANP(T2238C) | 57 | 3 | LTA(A252G) | 0.143 | 10.7 | 3.4 |
| 62 | 1 | Thrombospondin-1(A2210G) | 67 | 23 | von Willebrand Factor (G-1051A) | 0.415 | 56.9 | 3.2 |
| 58 | 1 | LTA(C804A(Thr26Asn)) | 65 | 12 | IL-18(C-607A) | 0.078 | 15.1 | 3.2 |
| 58 | 1 | LTA(C804A(Thr26Asn)) | 64 | 1 | interleukin 1 beta(C3953T) | 0.137 | 9.2 | 3.2 |
| 56 | 3 | resistin(ATG repeat) | 57 | 3 | LTA(A252G) | 0.137 | 9 | 3.2 |
| 36 | 12 | PAI-1 | 72 | 1 | PGC-1(G1302A(Thr394Thr)) | 0.041 | 7.9 | 3.2 |
| 35 | 23 | beta Fib(C148T) | 58 | 1 | LTA(C804A(Thr26Asn)) | 0.039 | 7.5 | 3.2 |
| 58 | 1 | LTA(C804A(Thr26Asn)) | 59 | 23 | Thrombospondin4(G1186C(Ala387Pro)) | 0.147 | 9.7 | 3.1 |
| 8 | 3 | TNFalfa(G-238A) | 58 | 1 | LTA(C804A(Thr26Asn)) | 0.147 | 9.4 | 3.1 |
| 35 | 23 | beta Fib(C148T) | 57 | 3 | LTA(A252G) | 0.039 | 7.3 | 3.1 |
| 26 | 23 | HANP(T2238C) | 58 | 1 | LTA(C804A(Thr26Asn)) | 0.145 | 9.2 | 3 |
| 14 | 23 | CRP(G1059C) | 58 | 1 | LTA(C804A(Thr26Asn)) | 0.144 | 9.1 | 3 |
| 53 | 1 | apolipoproteinE(E3 | 58 | 1 | LTA(C804A(Thr26Asn)) | 0.137 | 8.2 | 3 |
| | | inexon 4(Arg 158Cys) | | | | | | |
| 21 | 1 | AT2-receptor | 72 | 1 | PGC-1(G1302A(Thr394Thr)) | 0.044 | 7.9 | 3 |
| 20 | 1 | ACE | 44 | 23 | microsomal triglyceride transfer | 0.043 | 7.5 | 3 |
| | | | | | protein(G-493T) | | | |

In Tables 9-1 and 9-2, “Polymorphism No.” represents a genetic polymorphism having the same number in following Tables 10; the number of “Category” represents a genotype constituting the combination among genotypes of genetic polymorphisms, in which “1” represents homozygosis of the polymorphism having an anterior base in alphabetic order of substituted bases of genetic polymorphism; “2” represents heterozygosis of the polymorphism; “3” represents homozygosis of the polymorphism having a posterior base in alphabetic order of substituted bases of genetic polymorphism; “1 2” represents a genotype as a collection of 1 and 2; and “2 3” represents a genotype as a collection of 2 and 3 in the sites of genetic polymorphisms shown in Tables 10-1 and 10-2. Taking MMP-12 (A82G) as an example, A is anterior to G in alphabetic order of A and G which are substituted bases, thus “1” represents homozygosis of A, “2” represents heterozygosis of AG and 3″ represents homozygosis of G.

TABLE 10-1

| Polymorphism | | Name of Genetic Polymorphism | | |
|---|---|---|---|---|
| No. | Symbol | Name of Gene | site | ref. SNP ID |
| 1 | N1 | e NOS | T-786C + 4repeat | rs2070744 |
| 2 | NOS3 | e NOS | G894T(Glu298Asp) | rs1799983 |
| 6 | IL62 | Interleukin-6 | C-634G | rs1800796 |
| 7 | N10 | TGF -beta 1 | T29C(Leu10Pro) | 329th of AY330201 |
| 8 | TNFa2 | Tumor necrosis factor- α | G-308A | rs1800629 |
| 10 | MMP12 | MMP-12 | A-82G | rs2276109 |
| 11 | MCP1 | MCP-1(chemokine) | A-2518G | rs1024611 |
| 14 | CRP1 | C-reactive protein | G1059C | rs1800947 |
| 16 | ESL2 | E-selectin | A561C(Ser128Arg) | rs5361 |
| 17 | ICAM1 | intercellular adhesin molecule 1 | G/A(E469K) | rs5498 |
| 18 | ESL1 | E-selectin | G98T | rs1805193 |
| 20 | ACE ID | ACE | I/D type | 1451 to 1738th |
| | | | | deletion of X62855 |
| 21 | AGTR1-3 | ATI receptor | A1166C | rs5186 |
| 24 | β 2AR4 | β 2-Adrenergic Receptor | C79G | rs1042714 |
| 25 | β 2AR-1 | β 2-Adrenergic Receptor | A46G(Arg16Gly) | rs1042713 |
| 26 | HANP1 | Human Atrial Natriuuretic | T2238C | rs5065 |
| 27 | GP3A | GP IIB IIIa | C1565T (PIA2) | rs5918 |
| 29 | GP1ba | Human Platelet Antigen-2 | C1018T(Thr145Met) | rs6065 |
| 35 | FGB3 | beta Fibrinogen | C148T | rs1800787 |
| 36 | PAI | PAI-I | 4G/5G at -668 | rs1799889 |
| 37 | MTHFR | MTHFR | C677T | rs1801133 |
| 38 | ABCA1 | ABCA 1 | G1051A(Arg219Lys) | rs2230806 |
| 40 | PONA1 | PON1 | A584G(Gln192Arg) | rs662, (2003/8/7 |
| | | | | site correction 575→ |
| | | | | 584) |
| 41 | PAR2 | PPAR gamma | C/G(Pro12Ala) | rs1801282 |
| 42 | HL1 | hepatic lipase | C-480T | rs1800588 |
| 44 | MTP1 | microsomal trigyceride transfer | G-493T | rs1800591 |
| | | protein | | |
| 47 | S2AR | serotonin 2A receptor | T102C | rs6313 |
| 49 | MMP71 | matrilysin promoter | A-181G | 1022th of L22525 |
| 50 | N7 | p 22phox | C242T(His72Tyr) | rs4673 |
| 51 | CF12 | Factor XII | C46T(Arg 353Gln) | rs1801020 |
| 52 | GP1a1 | Glycoprotien I a | C807T | rs1126643 |
| 53 | APE3 | Apolipoprotein E ε 3 in exon 4 | C/T(Arg158Cy) | rs7412 |
| 54 | 1A2 | Glycoprotien I a | G873A | rs1062535 |
| 55 | BKR1 | bradykinin B2receptor | C-58T | rs1799722 |
| 56 | REG1 | Resistin | ATG 6 | rs3833230 |
| | | | repeat(1:6/6, 2:6/7, | |
| | | | 3:7/7, 4:7/8, 5:8/8) | |
| 57 | LTA1 | Lymphotoxin-alfa | A252G | 1069th of M16441 |
| 58 | LTA2 | Lymphotoxin-alfa | C804A(Thr26Asn) | rs1041981 |
| 59 | TS41 | Thrombospondin-4 | G1186C(Ala387Pro) | rs1866389 |
| 60 | APM12 | ADIPONECTIN | G276T | rs1501299, IMS- |
| | | | | JST013728 |

TABLE 10-2

| Polymorphism | | Name of Genetic Polymorphism | | |
|---|---|---|---|---|
| No. | Symbol | Name of Gene | site | ref. SNP ID |
| 61 | RAGE3 | RAGE | G/A(Gly82Ser ) | rs2070600 |
| 62 | TS11 | Thrombospondin-1 | A2210G(Asn700Ser) | 55322th of AC037198 |
| 63 | 2C9-3 | CYP2C9*3 | A1075C(Leu359Ile) | rs 1057910 |
| 64 | IL1B2 | IL-1 β | C3953T | rs 1143634 |
| 65 | IL-181 | Interleukin-18 | C-607A | rs 1946518 |
| 66 | MS1 | Methionine synthase | A2756G(Asp919Gly) | rs 1805087 |
| 67 | VWF2 | von Willebrand Factor | G-1051A | rs 7965413 |
| 68 | IL102 | Interleukin-10 | C-819T | rs 1800871 |
| 69 | IL-182 | Interleukin-18 | G-137C | rs 187238 |
| 70 | TPO1 | Thrombopoietin | A5713G | rs6141 |
| 71 | LRP1 | LDL receptor related protein | C766T | 516th of AF058399 |
| 72 | PGC11 | Peroxisome proliferation activated | G1302A(Thr394Thr) | rs2970847 |
| | | receptor γ coactivator-1(PGC-1) | | |

<6> A method for determining the risk of arteriosclerotic disease according to the above-mentioned <5>, wherein at least 50% of the genetic polymorphisms shown in Table 10-1 and Table 10-2, are used.

<7> A method for determining the risk of arteriosclerotic disease according to the above-mentioned <5>, wherein at least 90% of the genetic polymorphisms shown in Table 10-1 and 10-2, are used.

<8> A method for determining the risk of arteriosclerotic disease according to the above-mentioned <6>, wherein the combination of plural genetic polymorphisms further includes at least one set of arteriosclerosis-associated genetic polymorphisms selected from sets of arteriosclerosis-associated genetic polymorphisms shown in following Tables 1-1 to 1-4.

TABLE 1-1

| Polymorphism | | Polymorphism | | Polymorphism | | Polymorphism | |
|---|---|---|---|---|---|---|---|
| No. | Category | No. | Category | No. | Category | No. | Category |
| 3 | 12 | 46 | 12 | | | | |
| 1 | 3 | 21 | 1 | 28 | 12 | | |
| 1 | 3 | 28 | 12 | 29 | 1 | | |
| 1 | 3 | 28 | 12 | 40 | 12 | | |
| 1 | 3 | 28 | 12 | 34 | 23 | | |
| 3 | 12 | 4 | 1 | 7 | 23 | | |
| 3 | 12 | 7 | 23 | 17 | 12 | | |
| 3 | 12 | 7 | 2 3 | 31 | 12 | | |
| 3 | 12 | 7 | 23 | 34 | 12 | | |
| 3 | 12 | 8 | 3 | 46 | 12 | | |
| 3 | 12 | 9 | 12 | 46 | 12 | | |
| 3 | 12 | 12 | 23 | 31 | 12 | | |
| 3 | 12 | 12 | 23 | 46 | 12 | | |
| 3 | 12 | 13 | 23 | 46 | 12 | | |
| 3 | 12 | 14 | 23 | 46 | 12 | | |
| 3 | 12 | 15 | 12 | 25 | 12 | | |
| 3 | 12 | 15 | 12 | 46 | 12 | | |
| 3 | 12 | 22 | 12 | 31 | 12 | | |
| 3 | 12 | 24 | 12 | 46 | 12 | | |
| 3 | 12 | 25 | 12 | 31 | 12 | | |
| 3 | 12 | 26 | 3 | 46 | 12 | | |
| 3 | 12 | 28 | 23 | 31 | 12 | | |
| 3 | 12 | 28 | 23 | 46 | 12 | | |
| 3 | 12 | 29 | 12 | 46 | 12 | | |
| 3 | 12 | 30 | 12 | 46 | 12 | | |
| 3 | 12 | 31 | 12 | 41 | 1 | | |
| 3 | 12 | 41 | 1 | 42 | 12 | | |
| 3 | 12 | 41 | 1 | 46 | 12 | | |
| 3 | 12 | 43 | 23 | 46 | 12 | | |
| 3 | 12 | 44 | 12 | 46 | 12 | | |
| 3 | 12 | 42 | 12 | 47 | 23 | | |
| 3 | 12 | 46 | 12 | 49 | 12 | | |
| 4 | 1 | 7 | 12 | 28 | 12 | | |
| 4 | 1 | 28 | 12 | 40 | 12 | | |
| 4 | 23 | 25 | 1 | 40 | 12 | | |
| 6 | 1 | 7 | 1 | 20 | 1 | | |
| 7 | 1 | 37 | 3 | 45 | 1 | | |
| 11 | 3 | 20 | 3 | 32 | 12 | | |
| 11 | 1 | 35 | 1 | 38 | 3 | | |
| 11 | 1 | 38 | 3 | 40 | 12 | | |
| 17 | 1 | 36 | 12 | 37 | 3 | | |
| 17 | 1 | 37 | 3 | 48 | 1 | | |
| 17 | 23 | 28 | 12 | 40 | 12 | | |
| 20 | 3 | 22 | 1 | 24 | 23 | | |

TABLE 1-2

| Polymorphism | | Polymorphism | | Polymorphism | | Polymorphism | |
|---|---|---|---|---|---|---|---|
| No. | Category | No. | Category | No. | Category | No. | Category |
| 20 | 3 | 24 | 23 | 33 | 12 | | |
| 20 | 3 | 24 | 23 | 38 | 12 | | |
| 20 | 3 | 24 | 23 | 48 | 1 | | |
| 17 | 1 | 20 | 3 | 32 | 12 | | |
| 20 | 3 | 32 | 12 | 40 | 12 | | |
| 20 | 3 | 32 | 12 | 38 | 3 | | |
| 21 | 1 | 28 | 12 | 40 | 12 | | |
| 11 | 12 | 25 | 1 | 38 | 3 | | |
| 17 | 1 | 25 | 1 | 38 | 3 | | |
| 23 | 12 | 25 | 1 | 38 | 3 | | |
| 25 | 1 | 36 | 1 | 38 | 3 | | |
| 25 | 1 | 38 | 3 | 40 | 12 | | |
| 28 | 12 | 32 | 3 | 40 | 12 | | |
| 7 | 12 | 28 | 12 | 33 | 12 | | |
| 28 | 12 | 38 | 23 | 40 | 12 | | |
| 31 | 3 | 32 | 3 | 47 | 1 | | |
| 4 | 12 | 31 | 3 | 45 | 23 | | |
| 15 | 1 | 31 | 3 | 45 | 23 | | |
| 31 | 3 | 45 | 23 | 46 | 12 | | |
| 34 | 3 | 36 | 12 | 39 | 12 | | |
| 34 | 3 | 37 | 1 | 47 | 1 | | |
| 17 | 12 | 34 | 3 | 39 | 12 | | |
| 34 | 3 | 39 | 12 | 41 | 1 | | |
| 34 | 3 | 39 | 12 | 45 | 1 | | |
| 16 | 1 | 34 | 1 | 40 | 1 | | |
| 18 | 1 | 34 | 1 | 40 | 1 | | |
| 24 | 1 | 34 | 1 | 40 | 1 | | |
| 34 | 1 | 40 | 1 | 45 | 1 | | |
| 12 | 12 | 39 | 12 | 43 | 3 | | |
| 1 | 23 | 11 | 3 | 31 | 23 | 39 | 12 |
| 2 | 1 | 7 | 3 | 20 | 3 | 38 | 3 |
| 2 | 1 | 25 | 12 | 38 | 3 | 39 | 12 |
| 2 | 1 | 33 | 23 | 38 | 3 | 39 | 12 |
| 2 | 1 | 11 | 3 | 31 | 23 | 39 | 12 |
| 2 | 1 | 35 | 1 | 38 | 3 | 39 | 12 |
| 4 | 23 | 20 | 3 | 33 | 12 | 40 | 12 |
| 4 | 23 | 23 | 3 | 33 | 12 | 40 | 12 |
| 4 | 23 | 33 | 12 | 40 | 12 | 47 | 12 |
| 4 | 23 | 11 | 3 | 20 | 3 | 40 | 12 |
| 4 | 23 | 20 | 3 | 31 | 23 | 40 | 12 |
| 4 | 23 | 29 | 1 | 38 | 3 | 40 | 12 |
| 5 | 1 | 11 | 3 | 39 | 12 | 43 | 3 |
| 5 | 1 | 12 | 12 | 20 | 3 | 23 | 3 |
| 5 | 1 | 15 | 1 | 20 | 3 | 32 | 12 |

TABLE 1-3

| Polymorphism | | Polymorphism | | Polymorphism | | Polymorphism | |
|---|---|---|---|---|---|---|---|
| No. | Category | No. | Category | No. | Category | No. | Category |
| 5 | 1 | 20 | 3 | 23 | 3 | 31 | 3 |
| 5 | 1 | 20 | 3 | 37 | 23 | 39 | 12 |
| 5 | 1 | 12 | 12 | 25 | 1 | 44 | 1 |
| 5 | 1 | 25 | 1 | 39 | 12 | 45 | 1 |
| 6 | 12 | 11 | 3 | 37 | 23 | 39 | 12 |
| 6 | 12 | 11 | 3 | 39 | 12 | 43 | 3 |
| 6 | 1 | 15 | 1 | 17 | 3 | 25 | 1 |
| 6 | 1 | 17 | 3 | 33 | 12 | 40 | 12 |
| 6 | 1 | 11 | 3 | 20 | 1 | 42 | 12 |
| 6 | 1 | 20 | 1 | 23 | 3 | 40 | 12 |
| 6 | 23 | 9 | 1 | 22 | 23 | 25 | 1 |
| 6 | 23 | 22 | 23 | 25 | 1 | 42 | 12 |
| 6 | 23 | 20 | 3 | 25 | 1 | 46 | 12 |
| 7 | 3 | 9 | 12 | 20 | 3 | 38 | 3 |
| 7 | 3 | 13 | 3 | 20 | 3 | 38 | 3 |
| 7 | 3 | 14 | 3 | 20 | 3 | 38 | 3 |
| 7 | 3 | 20 | 3 | 22 | 12 | 38 | 3 |
| 7 | 3 | 20 | 3 | 23 | 3 | 38 | 3 |
| 7 | 3 | 20 | 3 | 27 | 3 | 38 | 3 |
| 7 | 3 | 20 | 3 | 29 | 1 | 38 | 3 |
| 7 | 3 | 20 | 3 | 30 | 1 | 38 | 3 |
| 7 | 3 | 20 | 3 | 33 | 12 | 40 | 12 |
| 7 | 3 | 20 | 3 | 35 | 1 | 38 | 3 |
| 7 | 3 | 20 | 3 | 38 | 3 | 49 | 12 |
| 7 | 23 | 17 | 23 | 33 | 1 | 40 | 12 |
| 9 | 1 | 12 | 12 | 34 | 3 | 36 | 12 |
| 9 | 1 | 20 | 3 | 31 | 3 | 36 | 12 |
| 9 | 1 | 31 | 3 | 33 | 12 | 40 | 12 |
| 9 | 1 | 12 | 12 | 24 | 1 | 34 | 3 |
| 9 | 12 | 25 | 1 | 39 | 12 | 45 | 1 |
| 9 | 12 | 25 | 1 | 39 | 12 | 46 | 12 |
| 11 | 3 | 16 | 1 | 31 | 23 | 39 | 12 |
| 11 | 3 | 17 | 12 | 31 | 23 | 39 | 12 |
| 11 | 3 | 18 | 1 | 31 | 23 | 39 | 12 |
| 11 | 3 | 22 | 12 | 31 | 23 | 39 | 12 |
| 11 | 3 | 23 | 23 | 31 | 23 | 39 | 12 |
| 11 | 3 | 31 | 23 | 36 | 12 | 39 | 12 |
| 11 | 3 | 39 | 12 | 42 | 12 | 43 | 3 |
| 11 | 3 | 39 | 12 | 43 | 3 | 47 | 12 |
| 12 | 12 | 20 | 3 | 23 | 3 | 36 | 12 |
| 12 | 12 | 23 | 3 | 32 | 12 | 41 | 1 |
| 12 | 12 | 40 | 12 | 42 | 1 | 47 | 12 |
| 12 | 12 | 25 | 1 | 34 | 12 | 44 | 1 |
| 13 | 3 | 25 | 12 | 38 | 3 | 39 | 12 |

TABLE 1-4

| Polymorphism | | Polymorphism | | Polymorphism | | Polymorphism | |
|---|---|---|---|---|---|---|---|
| No. | Category | No. | Category | No. | Category | No. | Category |
| 13 | 3 | 36 | 12 | 38 | 3 | 39 | 12 |
| 13 | 3 | 38 | 3 | 39 | 12 | 43 | 3 |
| 14 | 3 | 15 | 1 | 20 | 3 | 32 | 12 |
| 14 | 3 | 20 | 3 | 23 | 3 | 31 | 3 |
| 15 | 1 | 25 | 1 | 31 | 3 | 32 | 3 |
| 15 | 1 | 17 | 23 | 31 | 3 | 40 | 12 |
| 15 | 1 | 31 | 3 | 33 | 12 | 40 | 12 |
| 15 | 1 | 20 | 3 | 23 | 3 | 32 | 12 |
| 15 | 1 | 20 | 3 | 32 | 12 | 36 | 12 |
| 17 | 1 | 20 | 3 | 25 | 1 | 31 | 23 |
| 20 | 3 | 23 | 3 | 31 | 3 | 32 | 3 |
| 20 | 3 | 23 | 3 | 31 | 3 | 38 | 23 |
| 20 | 23 | 28 | 12 | 40 | 12 | 42 | 23 |
| 20 | 3 | 28 | 12 | 29 | 1 | 46 | 23 |
| 20 | 3 | 29 | 1 | 37 | 23 | 39 | 12 |
| 20 | 3 | 29 | 1 | 39 | 12 | 42 | 12 |
| 20 | 3 | 29 | 1 | 39 | 12 | 43 | 3 |
| 20 | 3 | 22 | 1 | 23 | 3 | 32 | 12 |
| 20 | 3 | 36 | 12 | 37 | 23 | 39 | 12 |
| 20 | 3 | 36 | 12 | 39 | 12 | 45 | 1 |
| 20 | 3 | 38 | 3 | 39 | 12 | 43 | 3 |
| 20 | 3 | 23 | 3 | 32 | 12 | 46 | 12 |
| 20 | 3 | 37 | 23 | 39 | 12 | 46 | 12 |
| 20 | 3 | 39 | 12 | 42 | 12 | 46 | 12 |
| 20 | 3 | 39 | 12 | 45 | 1 | 46 | 12 |
| 20 | 3 | 39 | 12 | 46 | 12 | 47 | 12 |
| 20 | 3 | 37 | 23 | 39 | 12 | 48 | 1 |
| 23 | 3 | 37 | 3 | 38 | 3 | 48 | 1 |
| 23 | 3 | 31 | 23 | 39 | 12 | 47 | 12 |
| 23 | 3 | 37 | 23 | 39 | 12 | 47 | 12 |
| 25 | 1 | 34 | 12 | 38 | 3 | 44 | 1 |
| 25 | 1 | 33 | 23 | 39 | 12 | 45 | 1 |
| 25 | 1 | 39 | 12 | 45 | 1 | 47 | 12 |
| 25 | 1 | 33 | 23 | 39 | 12 | 46 | 12 |
| 28 | 12 | 29 | 1 | 33 | 12 | 40 | 12 |
| 28 | 12 | 29 | 1 | 40 | 12 | 42 | 23 |
| 28 | 12 | 34 | 23 | 40 | 12 | 42 | 23 |
| 28 | 12 | 33 | 12 | 40 | 12 | 48 | 1 |
| 28 | 12 | 40 | 12 | 42 | 23 | 48 | 1 |
| 31 | 3 | 32 | 3 | 33 | 12 | 37 | 23 |
| 35 | 1 | 38 | 3 | 39 | 12 | 43 | 3 |
| 35 | 1 | 37 | 3 | 44 | 23 | 48 | 1 |
| 35 | 1 | 39 | 12 | 43 | 3 | 44 | 23 |
| 36 | 1 | 37 | 3 | 40 | 12 | 42 | 23 |

In Tables 1-1 to 1-4, “Polymorphism No.” represents a genetic polymorphism having the same number in following Tables 2-1 and 2-2; the number of “Category” represents a genotype constituting the combination among genotypes of genetic polymorphisms, in which “1” represents homozygosis of the polymorphism having an anterior base in alphabetic order of substituted bases of genetic polymorphism; “2” represents heterozygosis of the polymorphism; “3” represents homozygosis of the polymorphism having a posterior base in alphabetic order of substituted bases of genetic polymorphism; “1 2” represents a genotype as a collection of 1 and 2; and “2 3” represents a genotype as a collection of 2 and 3 in the names of genetic polymorphisms shown in Tables 2-1 and 2-2. Taking MMP-12 (A82G) as an example, A is anterior to G in alphabetic order of A and G which are substituted bases, thus “1” represents homozygosis of A, “2” represents heterozygosis of AG and 3″ represents homozygosis of G.

TABLE 2-1

| Polymorphism | | Name of Genetic Polymorphism | | | Reference |
|---|---|---|---|---|---|
| No. | Symbol | Name of Gene | site | ref. SNP ID | Number |
| 1 | N1 | e NOS | T-786C + 4repeat | rs2070744 | 1 |
| 2 | NOS3 | e NOS | G894T(Glu298Asp) | rs1799983 | |
| 3 | IRS1 | IRS-1 | G3494A(Gly971Arg) | rs1801278 | |
| 4 | GSY | glycogen synthase | A/G(M416V) | rs5447 | 2 |
| 5 | | p22phox | C242T | | 3 |
| 6 | IL62 | Interleukin-6 | C-634G | rs1800796 | |
| 7 | N10 | TGF -beta 1 | T29C(Leu10Pro) | 329th of | 4 |
| | | | | AY330201 | |
| 8 | TNFa2 | Tumor necrosis factor- α | G-308A | rs1800629 | 25 |
| 9 | MMP9 | MMP-9 = gelatinase B | C-1562T | rs3918242 | 5 |
| 10 | MMP12 | MMP-12 | A-82G | rs2276109 | 6 |
| 11 | MCP1 | MCP-1(chemokine) | A-2518G | rs1024611 | 7 |
| 12 | MPO | myeloperoxidase | G-463A | rs2333227 | 8 |
| 13 | FR1 | Flactalkine receptor CX3CR1 | G84635A(Val249Ile) | rs3732379 | |
| 14 | CRP1 | C-reactive protein | G1059C | rs1800947 | 26 |
| 15 | CX37 | connexin37(gap junction protein) | C1019T(Pro319Ser) | rs1764391 | 21 |
| 16 | ESL2 | E-selectin | A561C(Ser128Arg) | rs5361 | 9 |
| 17 | ICAM1 | intercellular adhesin molecule 1 | G/A(E469K) | rs5498 | |
| 18 | ESL1 | E-selectin | G98T | rs1805193 | 22 |
| 19 | DRD2 | Dopamine D2 receptor | C/G(Ser311Cys) | rs1801028 | |
| 20 | ACE ID | ACE | I/D type | 1451 to | 23 |
| | | | | 1738th | |
| | | | | deletion of | |
| | | | | X62855 | |
| 21 | AGTR1-3 | AT1 receptor | A1166C | rs5186 | |
| 22 | AGT1 | Angiotensinogen | T704C(M235T) | JST050962, | 10 |
| | | | | rs699 | |
| 23 | 3AR | beta 3 adreno receptor | T/C(Trp64Arg) | rs4994 | 27 |
| 24 | β 2AR4 | β 2-Adrenergic Receptor | C79G | rs1042714 | |
| 25 | β 2AR-1 | β 2-Adrenergic Receptor | A46G(Arg16Gly) | rs1042713 | |
| 26 | HANP1 | Human Atrial Natriuuretic Peptide | T2238C | rs5065 | |
| 27 | GP3A | GP IIB IIIa | C1565T (PIA2) | rs5918 | 24 |
| 28 | G1A3 | GP Ia | A1648G | rs1801106 | 11 |
| 29 | GP1ba | Human Platelet Antigen-2 | C1018T(Thr145Met) | rs6065 | 28 |
| 30 | GP61 | Glycoprotein VI | T/C(Ser219Pro) | rs1613662 | |

TABLE 2-2

| Polymorphism | | Name of Genetic Polymorphism | | | Reference |
|---|---|---|---|---|---|
| No. | Symbol | Name of Gene | site | ref. SNP ID | Number |
| 31 | | Glycoprotien I a | C807T | | 12 |
| 32 | Tbm3 | thrombomodulin | G33A | 1487th of | 13 |
| | | | | M74564 | |
| 33 | | Factor XII | C46T | rs1801020 | |
| 34 | FGA1 | alpha fibrinogen | A/G(Thr312Ala) | rs6050 | 29 |
| 35 | FGB3 | beta Fibrinogen | C148T | rs1800787 | 14 |
| 36 | PAI | PAI-I | 4G/5G at -668 | rs1799889 | 15 |
| 37 | MTHFR | MTHFR | C677T | rs1801133 | |
| 38 | ABCA1 | ABCA 1 | G1051A(Arg219Lys) | rs2230806 | 16 |
| 39 | PONA2 | HUMPONA | A172T(Met55Leu) | rs3202100 | |
| 40 | PONA1 | PON1 | A584G(Gln192Arg) | rs662, | |
| | | | | (2003/8/7 | |
| | | | | site correction | |
| | | | | 575→584) | |
| 41 | PAR2 | PPAR gamma | C/G(Pro12Ala) | rs1801282 | |
| 42 | HL1 | hepatic lipase | C-480T | rs1800588 | 17 |
| 43 | APE2 | Apolipoprotein E | T/C(Cys112Arg) | rs429358 | 30 |
| 44 | MTP1 | microsomal trigyceride | G-493T | rs1800591 | 31 |
| | | transfer protein | | | |
| 45 | LPL3 | Lipoprotein lipase | C/G(Ser 447 STOP) | rs328 | |
| 46 | ESRa1 | alfa estrogen receptor | T/C(P vuII) | rs2234693 | 18 |
| 47 | S2AR | serotonin 2A receptor | T102C | rs6313 | 19 |
| 48 | GCLM1 | glutamate-cysteine ligase, | C588T | 2670th of | 32 |
| | | modifier subunit | | U72210 | |
| 49 | MMP71 | matrilysin promoter | A-181G | 1022th of | 20 |
| | | | | L22525 | |

<9>A method for determining the risk of arteriosclerotic disease according to any one of the above-mentioned <1> to <4>, wherein the combination of plural genetic polymorphisms includes at least one set of arteriosclerosis-associated genetic polymorphisms selected from sets of arteriosclerosis-associated genetic polymorphisms shown in following Tables 1-1 to 1-4. In Tables 1-1 to 1-4, “Polymorphism No.” represents a genetic polymorphism having the same number in following Tables 2-1 and 2-2; the number of “Category” represents a genotype constituting the combination among genotypes of genetic polymorphisms, in which “1” represents homozygosis of the polymorphism having an anterior base in alphabetic order of substituted bases of genetic polymorphism; “2” represents heterozygosis of the polymorphism; “3” represents homozygosis of the polymorphism having a posterior base in alphabetic order of substituted bases of genetic polymorphism; “1 2” represents a genotype as a collection of 1 and 2; and “2 3” represents a genotype as a collection of 2 and 3 in the names of genetic polymorphisms shown in Tables 2-1 and 2-2.

<10> A method for determining the risk of arteriosclerotic disease according to the above-mentioned <9>, wherein at least 50% of the genetic polymorphisms shown in Tables 2-1 and 2-2, are used.

<11> A method for determining the risk of arteriosclerotic disease according to the above-mentioned <9>, wherein at least 90% of the genetic polymorphisms shown in Tables 2-1 and 2-2, are used.

<12> A method for determining the risk of arteriosclerotic disease according to any one of the above-mentioned <1> to <11>, wherein the combination of plural genetic polymorphisms is selected so that,
when cases having a carotid arterial intima-media thickness at least 0.2 mm larger than the average of carotid arterial intima-media thickness of healthy subjects are defined as arteriosclerotic disease cases and the other cases are defined as non-arteriosclerotic disease cases,
cases having a combination of plural genetic polymorphisms showing a significant positive correlation with the carotid arterial intima-media thickness occupy 30% or more of a population of arteriosclerotic disease cases comprising at least 150 cases, and cases having a combination of plural genetic polymorphisms showing a significant positive correlation with the carotid arterial intima-media thickness occupy 15% or less of a population of non-arteriosclerotic disease cases comprising at least 150 cases.

<13> A method for determining the risk of arteriosclerotic disease according to any one of the above-mentioned <1> to <12>, wherein the combination of plural genetic polymorphisms is selected so that,
when cases having a carotid arterial intima-media thickness at least 0.2 mm larger than the average of carotid arterial intima-media thickness of healthy subjects are defined as arteriosclerotic disease cases and the other cases are defined as non-arteriosclerotic disease cases,
cases having a combination of plural genetic polymorphisms showing a significant positive correlation with the carotid arterial intima-media thickness occupy 70% or more of a population of arteriosclerotic disease cases comprising at least 150 cases, and cases having a combination of plural genetic polymorphisms showing a significant positive correlation with the carotid arterial intima-media thickness occupy 35% or less of a population of non-arteriosclerotic disease cases comprising at least 150 cases.

<14> A method for determining the risk of arteriosclerotic disease according to the above-mentioned <12> or <13>, wherein the population of arteriosclerotic disease cases and the population of non-arteriosclerotic disease cases are each a population of patients with diabetes having no myocardial infarction clinical history.

<15> A method for determining the risk of arteriosclerotic disease according to any one of the above-mentioned <1> to <14>, wherein the combination of plural genetic polymorphisms is a combination of 5 or less of genetic polymorphisms.

<16> A method for determining the risk of arteriosclerotic disease according to any one of the above-mentioned <1> to <14>, wherein the combination of plural genetic polymorphisms comprises at least three genetic polymorphisms.

<17> A method for determining the risk of arteriosclerotic disease according to any one of the above-mentioned <1> to <16>, wherein the combination of plural genetic polymorphisms includes at least two genetic polymorphisms belonging to any of following groups a) to l):
a) group of genetic polymorphisms relating to the renin-angiotensin system;
b) group of genetic polymorphisms relating to the platelet function-coagulation system;
c) group of genetic polymorphisms relating to lipid;
d) group of genetic polymorphisms relating to nitrogen-monoxide synthase;
e) group of genetic polymorphisms relating to TNF-α gene;
f) group of genetic polymorphisms relating to IRS-1 gene;
g) group of genetic polymorphisms relating to FABP2 gene;
h) group of genetic polymorphisms relating to the muscle glycogen synthase gene;
i) group of genetic polymorphisms relating to NADP-NADPH oxidase p22phox;
j) group of genetic polymorphisms relating to methylenetetrahydrofolate reductase;
k) group of genetic polymorphisms relating to heat shock protein 70-1; and
l) group of genetic polymorphisms relating to TGF-β1 gene.

<18> A method for determining the risk of arteriosclerotic disease according to the above-mentioned <17>, wherein the combination of plural genetic polymorphisms includes at least a genetic polymorphism belonging to the group of genetic polymorphisms relating to the platelet function-coagulation system, and
a genetic polymorphism belonging to at least any of the group of genetic polymorphisms relating to the renin-angiotensin system, the group of genetic polymorphisms relating to methylenetetrahydrofolate reductase (MTHFR) and the group of genetic polymorphisms relating to lipid.

<19> A method for determining the risk of arteriosclerotic disease according to the above-mentioned <18>, wherein the genetic polymorphism belonging to the group of genetic polymorphisms relating to the platelet function-coagulation system is a genetic polymorphism relating to PAI-1 gene.

<20> A method for determining the risk of arteriosclerotic disease according to one of the above-mentioned <18> and <19>, wherein the genetic polymorphism belonging to the group of genetic polymorphisms relating to the renin-angiotensin system is a genetic polymorphism relating to ACE gene.

<21> A method for determining the risk of arteriosclerotic disease according to any one of the above-mentioned <18> and <19>, wherein the genetic polymorphism belonging to the group of genetic polymorphisms relating to lipid is a genetic polymorphism relating to HUMPOMA gene.

<22> A method for determining the risk of arteriosclerotic disease according to any one of the above-mentioned <1> to <21>, further comprising a risk evaluation process for evaluating the risk of arteriosclerosis caused by an environmental factor from the information of the subject on the environmental factor based on the risk of arteriosclerosis inherent to the environmental factor.

<23> A method for determining the risk of arteriosclerotic disease according to any one of the above-mentioned <1> to <22>, further comprising a risk evaluation process for evaluating the risk of arteriosclerosis depending on the carotid arterial intima-media thickness from the carotid arterial intima-media thickness of the subject based on the risk of arteriosclerosis inherent to the carotid arterial intima-media thickness.

<24> A method for determining the risk of arteriosclerotic disease according to any one of the above-mentioned <1> to <23>, comprising plural risk evaluation processes and further comprising a process for calculating the risk of arteriosclerotic disease by considering all the risks of arteriosclerosis determined in the individual risk evaluation processes.

<25> A method for determining the risk of arteriosclerotic disease according to any one of the above-mentioned <1> to <24>, further comprising a detection process for detecting the genotype of the subject on the plural genetic polymorphisms before a revealing process.

<26> A method for measuring the risk of arteriosclerotic disease comprising a detection process for detecting the genotype of a subject on the plural genetic polymorphisms, and
a risk evaluation process for evaluating the risk of arteriosclerosis caused by genetic polymorphisms from the genotype of the subject on the genetic polymorphisms detected in the detection process based on the risk of arteriosclerosis inherent to the combination of plural genetic polymorphisms,
wherein the combination of plural genetic polymorphisms includes at least one combination of genetic polymorphisms having a significant positive correlation with the carotid arterial intima-media thickness.

<27> A method for revealing an arteriosclerotic disease-associated factor, comprising a revealing process for revealing a set of arteriosclerosis-associated genetic polymorphisms in genetic polymorphisms of a subject by selectively uncovering the genotype of plural genetic polymorphisms relating to the set of arteriosclerosis-associated genetic polymorphisms among genetic polymorphisms of the subject,
wherein the set of arteriosclerosis-associated genetic polymorphisms is a combination of plural genetic polymorphisms having a significant positive correlation with the carotid arterial intima-media thickness and is an arteriosclerotic disease-associated factor.

<28> A method for revealing an arteriosclerotic disease-associated factor according to the above-mentioned <27>, wherein the revealing process is represented by whether or not the selectively uncovered genotype of plural genetic polymorphisms corresponds to the set of arteriosclerosis-associated genetic polymorphisms.

<29> A method for revealing an arteriosclerotic disease-associated factor according to the above-mentioned <27>, wherein the revealing process is represented by the odds ratio of having a significant positive correlation with the carotid arterial intima-media thickness inherent to the set of arteriosclerosis-associated genetic polymorphisms when the selectively uncovered genotype of plural genetic polymorphisms corresponds to the set of arteriosclerosis-associated genetic polymorphisms.

<30> A method for revealing an arteriosclerotic disease-associated factor according to the above-mentioned <27>, wherein the revealing process is represented by an an amount of increase in the carotid arterial intima-media thickness inherent to the set of arteriosclerosis-associated genetic polymorphisms when the selectively uncovered genotype of plural genetic polymorphisms corresponds to the set of arteriosclerosis-associated genetic polymorphisms.

<31> A method for revealing an arteriosclerotic disease-associated factor according to any one of the above-mentioned <27> to <30>, wherein the set of arteriosclerosis-associated genetic polymorphisms includes at least one set of arteriosclerosis-associated genetic polymorphisms selected from the sets of arteriosclerosis-associated genetic polymorphisms shown in Tables 1-1 to 1-4.

<32> A method for revealing an arteriosclerotic disease-associated factor according to any one of the above-mentioned <27> to <31>,
wherein the set of arteriosclerosis-associated genetic polymorphisms is selected so that,
when cases having a carotid arterial intima-media thickness at least 0.2 mm larger than the average of carotid arterial intima-media thickness of healthy subjects are defined as arteriosclerotic disease cases and the other cases are defined as non-arteriosclerotic disease cases,
cases having at least one set of arteriosclerosis-associated genetic polymorphisms occupy 30% or more of a population of arteriosclerotic disease cases comprising at least 150 cases, and cases having at least one set of arteriosclerosis-associated genetic polymorphisms occupy 15% or less of a population of non-arteriosclerotic disease cases comprising at least 150 cases.

<33> A method for revealing an arteriosclerotic disease-associated factor according to the above-mentioned <32>, wherein the population of arteriosclerotic disease cases and the population of non-arteriosclerotic disease cases are each a population of patients with diabetes having no myocardial infarction clinical history.

<34> A method for revealing an arteriosclerotic disease-associated factor according to any one of the above-mentioned <27> to <33>, wherein the set of arteriosclerosis-associated genetic polymorphisms comprises two to five of at least any of genetic polymorphisms.

<35> A method for revealing an arteriosclerotic disease-associated factor according to any one of the above-mentioned <27> to <34>, wherein the set of arteriosclerosis-associated genetic polymorphisms comprises at least three genetic polymorphisms.

<36> A method for revealing an arteriosclerotic disease-associated factor according to any one of the above-mentioned <27> to <35>, wherein the set of arteriosclerosis-associated genetic polymorphisms includes at least two genetic polymorphisms belonging to any of following groups a) to 1):
a) group of genetic polymorphisms relating to the renin-angiotensin system;
b) group of genetic polymorphisms relating to the platelet function-coagulation system;
c) group of genetic polymorphisms relating to lipid;
d) group of genetic polymorphisms relating to nitrogen-monoxide synthase;
e) group of genetic polymorphisms relating to TNF-α gene;
f) group of genetic polymorphisms relating to IRS-1 gene;
g) group of genetic polymorphisms relating to FABP2 gene;
h) group of genetic polymorphisms relating to the muscle glycogen synthase gene;
i) group of genetic polymorphisms relating to NADP-NADPH oxidase p22phox;
j) group of genetic polymorphisms relating to methylenetetrahydrofolate reductase;
k) group of genetic polymorphisms relating to heat shock protein 70-1; and
l) group of genetic polymorphisms relating to TGF-β1 gene.

<37> A method for revealing an arteriosclerotic disease-associated factor according to any one of the above-mentioned <27> to <36>, further comprising a detection process for detecting the genotype of the subject on the plural genetic polymorphisms before the revealing process.

<38> A method for detecting genetic polymorphisms comprising a process for detecting a genotype of a subject on genetic polymorphisms constituting at least one set of arteriosclerosis-associated genetic polymorphisms selected from the sets of arteriosclerosis-associated genetic polymorphisms shown in Tables 9-1 and 9-2,
wherein the detection result is used for determining the risk of arteriosclerotic disease.

<39> A method for detecting genetic polymorphisms comprising a process for detecting a genotype of a subject on genetic polymorphisms constituting at least one set of arteriosclerosis-associated genetic polymorphisms selected from the sets of arteriosclerosis-associated genetic polymorphisms shown in Tables 1-1 to 1-4,
wherein the detection result is used for determining the risk of arteriosclerotic disease.

<40> A genetic marker comprising genetic polymorphisms constituting at least one set of arteriosclerosis-associated genetic polymorphisms selected from
the sets of arteriosclerosis-associated genetic polymorphisms shown in Tables 9-1 and 9-2.

<41> A genetic marker comprising genetic polymorphisms constituting at least one set of arteriosclerosis-associated genetic polymorphisms selected from
the sets of arteriosclerosis-associated genetic polymorphisms shown in Tables 1-1 to 1-4.

<42> A kit for analyzing genetic polymorphisms comprising a pair of primers capable of specifically amplifying genes constituting at least one set of arteriosclerosis-associated genetic polymorphisms selected from the sets of arteriosclerosis-associated genetic polymorphisms shown in Tables 9-1 and 9-2 or a nucleic acid probe capable of specifically hybridizing the genes,
wherein the kit is so configured as to detect at least one of the sets of arteriosclerosis-associated genetic polymorphisms shown in Tables 9-1 and 9-2.

<43> A kit for anlalyzing genetic polymorphisms according to the above-mentioned <42>, wherein the sets of arteriosclerosis-associated genetic polymorphisms comprises at least 50% of the genetic polymorphisms shown in Tables 10-1 and 10-2.

<44> A kit for anlalyzing genetic polymorphisms according to the above-mentioned <42>, wherein the sets of arteriosclerosis-associated genetic polymorphisms comprises at least 90% of the genetic polymorphisms shown in Tables 10-1 and 10-2.

<45> A kit for analyzing genetic polymorphisms according to any one of the above-mentioned <42> to <44>, comprising a primer or a probe for detecting at least two genetic polymorphisms selected from the genetic polymorphisms shown in Tables 10-1 and 10-2,
when cases having a carotid arterial intima-media thickness at least 0.2 mm larger than the average of carotid arterial intima-media thickness of healthy subjects are defined as arteriosclerotic disease cases and the other cases are defined as non-arteriosclerotic disease cases,
cases having a combination of genotypes having a significant positive correlation with the carotid arterial intima-media thickness occupy 70% or more of a population of arteriosclerotic disease cases comprising at least 150 cases, and
occupy 35% or less of a population of non-arteriosclerotic disease cases comprising at least 150 cases, the combination of genotypes being in sets of arteriosclerosis-associated genetic polymorphisms shown in Tables 9-1 to 9-2 which are capable of including the selected genotypes.

<46> A kit for analyzing genetic polymorphisms comprising a pair of primers capable of specifically amplifying genes constituting at least one set of arteriosclerosis-associated genetic polymorphisms selected from the sets of arteriosclerosis-associated genetic polymorphisms shown in Tables 1-1 to 1-4 or a nucleic acid probe capable of specifically hybridizing the genes,
wherein the kit is so configured as to detect at least one of the sets of arteriosclerosis-associated genetic polymorphisms shown in Tables 1-1 to 1-4.

<47> A kit for anlalyzing genetic polymorphisms according to the above-mentioned <46>, wherein the sets of arteriosclerosis-associated genetic polymorphisms comprises at least 50% of the genetic polymorphisms shown in Tables 2-1 and 2-2.

<48> A kit for anlalyzing genetic polymorphisms according to the above-mentioned <46>, wherein the sets of arteriosclerosis-associated genetic polymorphisms comprises at least 90% of the genetic polymorphisms shown in Tables 2-1 and 2-2.

<49> A kit for analyzing genetic polymorphisms according to any one of the above-mentioned <46> to <48>, comprising at least any of primers and probes for detecting at least two genetic polymorphisms selected from the genetic polymorphisms shown in Tables 2-1 and 2-2,
wherein, when cases having a carotid arterial intima-media thickness at least 0.2 mm larger than the average of carotid arterial intima-media thickness of healthy subjects are defined as arteriosclerotic disease cases and the other cases are defined as non-arteriosclerotic disease cases,
cases having at least one combination of genotypes having a significant positive correlation with the carotid arterial intima-media thickness occupy 30% or more of a population of arteriosclerotic disease cases comprising at least 150 cases, and occupy 15% or less of a population of non-arteriosclerotic disease cases comprising at least 150 cases, the combination of genotypes being in sets of arteriosclerosis-associated genetic polymorphisms shown in Tables 1-1 to 1-4 which are capable of including the selected genotypes.

<50> A kit for analyzing genetic polymorphisms according to any one of the above-mentioned <42> to <49>, comprising plural primers or probes for detecting genetic polymorphisms belonging to different ones of following groups a) to l):
a) group of genetic polymorphisms relating to the renin-angiotensin system;
b) group of genetic polymorphisms relating to the platelet function-coagulation system;
c) group of genetic polymorphisms relating to lipid;
d) group of genetic polymorphisms relating to nitrogen-monoxide synthase;
e) group of genetic polymorphisms relating to TNF-α gene;
f) group of genetic polymorphisms relating to IRS-1 gene;
g) group of genetic polymorphisms relating to FABP2 gene;
h) group of genetic polymorphisms relating to the muscle glycogen synthase gene;
i) group of genetic polymorphisms relating to NADP-NADPH oxidase p22phox;
j) group of genetic polymorphisms relating to methylenetetrahydrofolate reductase;
k) group of genetic polymorphisms relating to heat shock protein 70-1; and
l) group of genetic polymorphisms relating to TGF-β1 gene.

<51> A kit for analyzing genetic polymorphisms according to the above-mentioned <42> to <50>, wherein the set of arteriosclerosis-associated genetic polymorphisms includes at least three genetic polymorphisms.

<52> An array for determining the risk of arteriosclerotic disease comprising a probe for detecting genetic polymorphisms constituting at least one set of arteriosclerosis-associated genetic polymorphisms selected from the sets of arteriosclerosis-associated genetic polymorphisms shown in Tables 9-1 and 9-2.

<53> An array for determining the risk of arteriosclerotic disease comprising a probe for detecting genetic polymorphisms constituting at least one set of arteriosclerosis-associated genetic polymorphisms selected from the sets of arteriosclerosis-associated genetic polymorphisms shown in Tables 1-1 to 1-4.

<54> An apparatus for determining the risk of arteriosclerotic disease using a computer, comprising:
a data table on the risk of arteriosclerosis in which combinations of plural genetic polymorphisms are listed with corresponding risks of arteriosclerosis, and
detection means for checking inputted combinations of plural genetic polymorphisms in a subject against the combinations of plural genetic polymorphisms in the data table on the risk of arteriosclerosis and, when there is a combination of genetic polymorphisms matching between the two, detecting the risk of arteriosclerosis corresponding to the combination of genetic polymorphisms.

<55> An apparatus for determining the risk of arteriosclerotic disease according to the above-mentioned <54>, wherein 1 unit is coordinated as the risk of arteriosclerosis with a combination of plural genetic polymorphisms having a significant positive correlation with the carotid arterial intima-media thickness in the data table on the risk of arteriosclerosis in which the combinations of plural genetic polymorphisms are listed with corresponding risks of arteriosclerosis.

<56> An apparatus for determining the risk of arteriosclerotic disease according to the above-mentioned <54>, wherein an odds ratio for the carotid arterial intima-media thickness of exceeding a normal range is coordinated as the risk of arteriosclerosis with a combination of plural genetic polymorphisms having a significant positive correlation with the carotid arterial intima-media thickness in the data table on the risk of arteriosclerosis in which the combination of plural genetic polymorphisms are listed with corresponding risks of arteriosclerosis.

<57> An apparatus for determining the risk of arteriosclerotic disease according to the above-mentioned <54>, wherein an increase in carotid arterial intima-media thickness is coordinated as the risk of arteriosclerosis with a combination of plural genetic polymorphisms having a significant positive correlation with the carotid arterial intima-media thickness in the data table on the risk of arteriosclerosis in which the combination of plural genetic polymorphisms are listed with corresponding risks of arteriosclerosis.

<58> An apparatus for determining the risk of arteriosclerotic disease according to any one of the above-mentioned <54> to <57>, wherein a combination of genetic polymorphisms is defined to have a significant positive correlation with the carotid arterial intima-media thickness when the odds ratio for the carotid arterial intima-media thickness of exceeding a normal range stands at a specific level or more and/or when the average of the carotid arterial intima-media thickness shows a significant difference.

<59> An apparatus for determining the risk of arteriosclerotic disease according to any one of the above-mentioned <54> to <58>, further comprising:
a data table on the risk of arteriosclerosis in which the presence or absence of, or the numerical value of an environmental factor is listed with a corresponding risk of arteriosclerosis; and
detection means for checking an inputted presence or absence of, or the numerical value of an environmental factor of a subject against the presence or absence of, or the numerical value of an environmental factor in the data table on the risk of arteriosclerosis, and detecting a risk of arteriosclerosis corresponding to the presence or absence of, or the numerical value of the environmental factor.

<60> An apparatus for determining the risk of arteriosclerotic disease according to any one of the above-mentioned <54> to <59>, comprising plural detection means and further comprising determination means for determining the risk of arteriosclerotic disease based on the additional value of plural risks of carotid artery detected by the plural detection means.

<61> An apparatus for determining the risk of arteriosclerotic disease according to any one of the above-mentioned <54> to <60>, further comprising:
a data table on the risk of arteriosclerosis in which a carotid arterial intima-media thickness is listed with a corresponding risk of arteriosclerosis, and
detection means for checking an inputted carotid arterial intima-media thickness of a subject against the carotid arterial intima-media thickness in the data table on the risk of arteriosclerosis and detecting a risk of arteriosclerosis corresponding to the carotid arterial intima-media thickness.

<62> An apparatus for determining the risk of arteriosclerotic disease according to any one of the above-mentioned <54> to <61>, further comprising determination means for determining the risk of arteriosclerotic disease based on an additional value of the inputted carotid arterial intima-media thickness of the subject with one of the carotid artery risk detected by the detection means and an additional value of carotid artery risks extracted by the plural extraction means.

<63> An apparatus for determining the risk of arteriosclerotic disease according to any one of the above-mentioned <54> to <62>, comprising vascular membrane thickness measuring means for measuring the carotid arterial intima-media thickness of a subject and supplying the carotid arterial intima-media thickness to the computer.

<64> A recording medium readable by computer, comprising a program for determining the risk of arteriosclerotic disease recorded thereon,
wherein the program comprises the steps of:
checking inputted combinations of plural genetic polymorphisms of a subject against the combinations of plural genetic polymorphisms in a data table on the risk of arteriosclerosis in which combinations of plural genetic polymorphisms are listed with corresponding risks of arteriosclerosis, the data table being recorded in a computer; and
detecting the risk of arteriosclerosis corresponding to the combination of genetic polymorphisms when there is a combination of genetic polymorphisms matching between the two as a result of checking.

<65> A recording medium according to the above-mentioned <64>, wherein 1 unit is coordinated as the risk of arteriosclerosis with a combination of plural genetic polymorphisms having a significant positive correlation with the carotid arterial intima-media thickness in the data table on the risk of arteriosclerosis in which the combinations of plural genetic polymorphisms are listed with corresponding risks of arteriosclerosis.

<66> A recording medium according to the above-mentioned <64>, wherein an odds ratio for the carotid arterial intima-media thickness of exceeding a normal range is coordinated as the risk of arteriosclerosis with a combination of plural genetic polymorphisms having a significant positive correlation with the carotid arterial intima-media thickness in the data table on the risk of arteriosclerosis in which the combinations of plural genetic polymorphisms are listed with corresponding risks of arteriosclerosis.

<67> A recording medium according to the above-mentioned <64>, wherein an increase in the carotid arterial intima-media thickness is coordinated as the risk of arteriosclerosis with a combination of plural genetic polymorphisms having a significant positive correlation with the carotid arterial intima-media thickness in the data table on the risk of arteriosclerosis in which the combinations of plural genetic polymorphisms are listed with corresponding risks of arteriosclerosis.

<68> A recording medium according to any one of the above-mentioned <64> to <67>, wherein a combination of genotypes is defined to have a significant positive correlation with the carotid arterial intima-media thickness when the odds ratio for the carotid arterial intima-media thickness of exceeding a normal range stands at a specific level or more and/or when the average of the carotid arterial intima-media thickness shows a significant difference.

<69> A recording medium according to any one of the above-mentioned <64> to <68>, wherein, when cases having a carotid arterial intima-media thickness at least 0.2 mm larger than the average of carotid arterial intima-media thickness of healthy subjects are defined as arteriosclerotic disease cases and the other cases are defined as non-arteriosclerotic disease cases,
cases having at least 1 unit of the risk of arteriosclerosis occupy 30% or more of a population of arteriosclerotic disease cases comprising at least 150 cases, and
the cases having at least 1 unit of the risk of arteriosclerosis occupy 15% or less of a population of non-arteriosclerotic disease cases comprising at least 150 cases.

### BRIEF DESCRIPTION OF THE DRAWINGS

FIG. 1 illustrates the ratio of adopted cases based on the number of genetic polymorphisms constituting a combination in Example 4.

### DESCRIPTION OF THE PREFERRED EMBODIMENTS

(Method for Determining the Risk of Arteriosclerotic Disease)

The method for determining the risk of arteriosclerotic disease of the present invention comprises a risk evaluation process for evaluating the risk of arteriosclerosis caused by genetic polymorphisms from genotype of a subject on the genetic polymorphisms, based on the risk of arteriosclerosis inherent to the combination of plural genetic polymorphisms. In the method, the combination of plural genetic polymorphisms includes at least one combination of plural genetic polymorphisms having a significant positive correlation with the carotid arterial intima-media thickness. The method can further comprise any other process as long as it comprise this process.

The arteriosclerotic disease refers to an ischaemic disease and includes cardiac angina, myocardial infarction, cerebral infarction and peripheral arterial obstruction. The risk of arteriosclerotic disease is an indicator which indicates the tendency to develop an arteriosclerotic disease or tendency for the advance of the disease.

The genetic polymorphism means a diversity in which plural allelic genes (alleles) lie in one gene locus. The “gene” used herein, however, is not limited to a region which will be transcribed as an RNA but includes all DNAs that can be specified in the human genome, including control regions such as promoter or enhancer. Of human genome DNAs, 99.9% are in common between individuals, and the residual 0.1% causes such a diversity and relates to a sensibility to a specific disease and individual difference in responsiveness to a medicament or environmental factor. If there is a genetic polymorphism, the phenotype does not always show difference. SNP (single nucleotide polymorphism) is one of the genetic polymorphisms, but genetic polymorphisms in the present invention are not limited to this.

Genetic polymorphisms for use in the present invention can be appropriately selected from known genetic polymorphisms which are supposed to be involved in the arteriosclerotic disease so as to satisfy following requirements for a plurality of genetic polymorphisms.

The combination of plural genetic polymorphisms for use in the present invention preferably includes at least one set of arteriosclerosis-associated genetic polymorphisms selected from the sets of arteriosclerosis-associated genetic polymorphisms shown in Tables 1-1 to 1-4. Tables 1-1 to 1-4 show the results of the following procedures, which will be illustrated in detail in Example 4. Specifically, a case control study was carried out in a population of patients with diabetes having no myocardial infarction clinical history on about 437 cases with early arteriosclerosis while taking about 195 cases without early arteriosclerosis as control. In this study, the significance level was set at an odds ratio of 10 or more and a chi-square of 6.635 (P<0.01) or more, and combinations of plural genetic polymorphisms having a positive correlation with the carotid arterial intima-media thickness were extracted. Each line represents a set of arteriosclerosis-associated genetic polymorphisms. The plurality of genetic polymorphisms for use in the present invention may include any of the sets of arteriosclerosis-associated genetic polymorphisms shown in Tables 1-1 to 1-4 and further include other genetic polymorphisms.

The combination of plural genetic polymorphisms for use in the present invention also preferably includes at least one set of arteriosclerosis-associated genetic polymorphisms selected from the sets of arteriosclerosis-associated genetic polymorphisms shown in Tables 9-1 to 9-2. Tables 9-1 to 9-2 show the results of the following procedures, which will be illustrated in detail in Example 5.

The plurality of genetic polymorphisms for use in the present invention can also be genetic polymorphisms listed in Table 3. References for the genetic polymorphisms listed in Table 3 are shown in Table 4. In Tables 3 and 4, the same number is applied to the same genetic polymorphism.

TABLE 3

| | | | | | Allele Type Serving as Risk | Amino Acid |
|---|---|---|---|---|---|---|
| No. | Associated Factor | Symbol | Cyto | Site | Factor | Variation |
| | Genetic polymorphisms relating to nitrogen monoxide synthase | | | | | |
| 1 | eNOS | NOS3 | 7q36 | Exon 7 | Type D (Glu298 → Asp) | GLu298 → Asp |
| 2 | eNOS | NOS3 | 7q36 | 5′ noncoding | Type C (T-786 → C) | |
| | | | | region | | |
| 3 | eNOS | NOS3 | 7q36 | Intron 4 | 4 repeat type (4 repeats → 5 | |
| | | | | | repeats) | |
| | Genetic polymorphisms relating to renin-angiotensin system | | | | | |
| 4 | ACE | ACE | 17q23 | Intron 16 | Type D [ I, D (I: insertion | |
| | | | | | type, D: deletion type) ] | |
| 5 | angiotensinogen | AGT | 1q42-q43 | Exon 2 | Type T (Met 235 → Thr) | Met235Thr |
| 6 | AT1 R | AGTR1 | 3q21-q25 | 3′ noncoding | Type C (A-1.166→ C) | |
| | | | | region | | |
| | Genetic polymorphisms relating to the platelet function-coagulation system | | | | | |
| 7 | GP Ib, ′ | GP1BA | 17pter-p 12 | Iba chain | Type M (Thr 145 → Met) | Thr145Met |
| 8 | GP′b, ′a | ITGB3 | 17q21.32 | III a chain | Type A2 (P1:A1 → A2) | |
| 9 | PAF acetyl hydrolase | LOC130273 | 2q35 | exon 9 | Type T (G 994′ T) | |
| 10 | coagulation factor VII | F7 | 13q34 | Intron 4 | H7H5 (H7, H6, H5) | |
| 11 | coagulation factor VII | F7 | 13q34 | exon 8 | G353 → A | Type R (Arg → |
| | | | | | | Gln) |
| 12 | Coagulation factor VIII | F8 | Xq28 | exon 2 | Type G (G, T) | |
| | subunit A | | | | | |
| 13 | coagulation factor XII | F12 | 5q33-qter | 5′-noncoding | Type C (C46 → T) | |
| | | | | region | | |
| 14 | fibrinogen | FGB | 4q28 | β-chain | Type A (G-455′ A) | |
| 15 | PAI-1 | SERPINE1 | 7q21.3-q22 | promoter | Type 4G (4G, 5G) | |
| | Genetic polymorphisms relating to lipid | | | | | |
| 16 | apoprotein E | APOE | 19q13.2 | exon 4 | Type E (E2,E3,E4) | |
| 17 | apoprotein A | APOA1 | 11q23-q24 | Kringle′ | Type T (Met 66 → Thr) | Met66Thr |
| 18 | | APOA2 | 1q21-q23 | | | |
| 19 | | APOA4 | 11q23 | | | |
| 20 | | APOAV | 11q23 | | | |
| 21 | apoprotein B | APOB | 2p24-p23 | | X+ type (X+, X−) | |
| 22 | LPL | LPL | 8p22 | exon 9 | Type X (Ser 477 → stop) | Ser477Ter |
| 23 | CETP | CEPT | 16q21 | intron 1 | B1 type (B1, B2) | |
| 24 | HUMPONA | PON1 | 7q21.3 | codon 192 | Arg type (Glul92 → Arg) | Glu192 → Arg |
| 25 | | | | | | Met55 → Leu |
| | Other factors | | | | | |
| 26 | heat shock protein 70-1 | HSPA1A | 6q21.3 | promoter | Type A (C-110 → A) | |
| 27 | | HSPA1B | 6q21.3 | | | |
| 28 | TNF-a | TNF | 6q21.3 | 5′-noncoding | Type A (G-308 → A) | |
| | | | | region | | |
| 29 | p22phox | LOC113690 | 16q24.2 | exon 4 | C242 → T | His72 → Tyr |
| 30 | MTHFR | MTHFR | 1q36.3 | | Val type (polymorphism of | Ala → Val |
| | | | | | Ala → Val by C677 → T) | |
| 31 | TGF-β1 | TGFB1 | 19q13.1 | exon 1 | T29 → C | Leu10Pro |
| 32 | | | | exon 1 | G74 → C | Arg25Pro |
| 33 | IRS-1 | IRS1 | 2q36 | | codon 972 | Gly972Arg |
| 34 | glycogen synthase | GYS1 | | exon 10 | | Met416Val |
| 35 | fatty acid binding | FABP2 | 4q28_q31 | | codon 54 | Ala54Thr |
| | protein | | | | | |

TABLE 4

| | Reference 1: polymorphism | Reference 2: relation with |
|---|---|---|
| No. | and activity | arteriosclerosis |
| 1 | J Am Coll Cardiol 1998; 31: | J Am Coll Cardiol 1998; |
| | 1506-10 | 31: 1506-10 |
| 2 | Circulation 1999; 99: 2864-70 | Circulation 1999; 99: 2864-70 |
| 3 | | |
| 4 | | |
| 5 | | |
| 6 | Hypertension 1994; 24: 63-9 | Lancet 1994; 344: 910-3 With |
| | | ACE-DD |
| 7 | Blood 1992; 79: 3086-90 | |
| 8 | | N Engl J Med 1996; 334: 1090-4 |
| 9 | | |
| 10 | | |
| 11 | Arterioscler Theromb 1991; | N Engl J Med 1998; 338: 79-85 |
| | 11: 540-6 | |
| 12 | | |
| 13 | | |
| 14 | | |
| 15 | Arterioscler Thromb 1991; | Proc Natl Acad Sci USA 1995; |
| | 11: 183-90 | 92: 1851-5 |
| 16 | Atherosclerosis 1988; 8: 1-21 | Lancet 1992; 340: 879-80 |
| 17 | | |
| 18 | | |
| 19 | | |
| 20 | | |
| 21 | | Lancet 1988; 2: 1442-5 |
| 22 | | |
| 23 | Atherosclerosis 1994; | N Engl J Med 1998; 338: 86-93 |
| | 110: 35-44 | |
| 24 | | |
| 25 | Nat Genetics 1993; 3: 73-6 | Metabolism 2001; 50: 1095-101 |
| 26 | | |
| 27 | | |
| 28 | | Atherosclerosis 2001; 154: 691-7 |
| | | negative |
| 29 | Am J Hum Genet 1992; 51: | Circulation 1998; 97: 135-7 |
| | 1127-35 | |
| 30 | Nat Genetics 1995; 10: 111-3 | Circulation 1997; 95: 21-3 |
| 31 | Nucleic Acid Res 1987; | Circulation 2000; 101: 2783-7 |
| | 15: 3188-9 | |
| 32 | Nucleic Acid Res 1987; | Hypertension 1996; 28: 881-7 |
| | 15: 3188-9 | |
| 33 | Lancet 1993; 342: 828-32 | Arterioscler Thromb Vasc Biol |
| | | 1999; 19: 2975-80 |
| 34 | Diabetologia 1997; 40: 947-52 | Diabetologia 1997; 40: 947-52 |
| 35 | J Clin Invest 1995; 95: 1281-7 | J Clin Invest 1995; 95: 1281-7 |

The “plural genetic polymorphisms” refer to two or more genetic polymorphisms in different gene loci. When they comprise two different genetic polymorphisms, they refer to, for example, SERPINE1 and ACE shown in Table 3, and when they comprise three different genetic polymorphisms, they refer to, for example, SERPINE1, APOA1 and APOA2.

The “combination of genetic polymorphisms” refers to a combination of genotypes of the plural genetic polymorphisms. For example, SERPINE1 polymorphism shown in Table 3 is a polymorphism of a promoter site with PAI-1 as an associated factor, and includes allelic genes, Type 4G and Type 5G, which have different numbers of repetitive G. Among them, Type 4G acts as a risk factor. The genotype of a subject on this SERPINE1 polymorphism stands as any of 4G/4G, 4G/5G and 5G/5G. Likewise, ACE polymorphism shown in Table 3 is a polymorphism of intron 16 with ACE as an associated factor and includes allelic genes, insertion type (Type I) and deletion type (Type D). Among them, Type D acts as a risk factor. The genotype of a subject on this ACE polymorphism stands any of D/D, D/I and I/I. When SERPINE1 polymorphism and ACE polymorphism are selected as the plural genetic polymorphisms, combinations of genetic polymorphisms on the two polymorphisms are a total of nine cases such as one having 4G/4G and D/D and one having 4G/4G and D/I. The risk can be set on each of the nine cases. However, the genotypes of the subject on SERPINE1 polymorphism, for example, can be classified as 4G/4G group having the risky allelic gene homozygously and other 5G/? group having 4G and 5G heterozygously or having 5G homozygously. Likewise, on ACE polymorphism, the genotypes are classified as D/D group and I/? group. Then, combinations of genotypes on the two polymorphisms are integrally classified as one having 4G/4G and D/D, one having 4G/4G and I/?, one having 5G/? and D/D, and one having 5G/? and I/? as combinations of genetic polymorphisms. The combinations of genotypes may further be classified as two groups, i.e., a combination of two genotypes each having the genotype homozygously(the combination of 4G/4G and D/D), and other combinations, and the risk can be set on each group. The combinations may be classified under a predetermined rule and the risk can be set on each group, as described in detail in Example 4. Such classification is not specifically limited.

The combination of plural genetic polymorphisms must comprise at least one combination of plural genetic polymorphisms having a significant positive correlation with the carotid arterial intima-media thickness.

For example, the combination of 4G/4G, Val/Val and D/D on SERPINE1 polymorphism with PAI-1 as an associated factor, MTHFR polymorphism with MTHFR as an associated factor, and ACE polymorphism with ACE as an associated factor has a significant positive correlation (relation in a direction to increase the carotid arterial intima-media thickness) with the carotid arterial intima-media thickness. Thus, one including this combination of plural genetic polymorphisms satisfies the requirement.

The measurement value of the carotid arterial intima-media thickness used herein is used for determining whether or not one has a significant correlation and is a measurement value measured with a high-resolution ultrasonic tomography apparatus. The carotid arterial intima-media thickness may be any one of the average thickness (IMT) as an average of carotid arterial intima-media thickness in one subject measured and the maximum thickness (PIMT) as a maximum of the carotid arterial intima-media thickness in one subject measured.

One is defined to have a significant relation in at least one of the case where the result in a statistically general hypothesis testing with a significance level of 0.05 is significant, the case where the increment in average thickness of carotid arterial intima media complex (ΔIMT) as an empiric value of significance is 0.2 mm or more in a multiple regression analysis, and the case where the increment in maximum thickness of carotid arterial intima media complex (ΔPIMT) as an empiric value of significance is 0.3 mm or more in a multiple regression analysis.

On the selection of the sets of plural genetic polymorphisms and the number of sets, the combination of plural genetic polymorphisms is preferably selected in the following manner. When cases having a carotid arterial intima-media thickness at least 0.2 mm larger than the average of carotid arterial intima-media thickness of healthy subjects are defined as arteriosclerotic disease cases and the other cases are defined as non-arteriosclerotic disease cases, cases having a combination of plural genetic polymorphisms showing a significant positive correlation with the carotid arterial intima-media thickness occupy 30% or more, preferably 50% or more, and further preferably 60% or more, of a population of arteriosclerotic disease cases comprising at least 150 cases, and cases having a combination of plural genetic polymorphisms showing a significant positive correlation with the carotid arterial intima-media thickness occupy 15% or less of a population of non-arteriosclerotic disease cases comprising at least 150 cases.

The combination of plural genetic polymorphisms is also preferably selected so that, under the same conditions, cases having a combination of plural genetic polymorphisms showing a significant positive correlation with the carotid arterial intima-media thickness occupy 70% or more of a population of arteriosclerotic disease cases comprising at least 150 cases, and cases having a combination of plural genetic polymorphisms showing a significant positive correlation with the carotid arterial intima-media thickness occupy 35% or less of a population of non-arteriosclerotic disease cases comprising at least 150 cases.

The number of genetic polymorphisms constituting the combination of plural genetic polymorphisms is preferably from about two to about five. A combination comprising six or more genetic polymorphisms can be used, but such a combination may possibly invite an increased error in the risk, because the percentage covered by the total of the combination in arteriosclerotic disease cases does not so increase, and the percentage in a control having the combination tends to increase. With an increasing number of polymorphisms constituting the combination, combinations having a significance remarkably increases, but the incidence in each combination markedly decreases. Thus, analysis may become complicated.

The combination can also be selected from combinations comprising at least three genetic polymorphisms.

The plural genetic polymorphisms preferably includes two or more, more preferably three or more, genetic polymorphisms belonging to any of the following groups a) to l):
a) group of genetic polymorphisms relating to the renin-angiotensin system;
b) group of genetic polymorphisms relating to the platelet function-coagulation system;
c) group of genetic polymorphisms relating to lipid;
d) group of genetic polymorphisms relating to nitrogen-monoxide synthase;
e) group of genetic polymorphisms relating to TNF-α gene;
f) group of genetic polymorphisms relating to IRS-1 gene;
g) group of genetic polymorphisms relating to FABP2 gene;
h) group of genetic polymorphisms relating to the muscle glycogen synthase gene;
i) group of genetic polymorphisms relating to NADP-NADPH oxidase p22phox;
j) methylenetetrahydrofolate reductase (MTHFR)-related genetic polymorphisms group;
k) group of genetic polymorphisms relating to heat shock protein 70-1; and
l) group of genetic polymorphisms relating to TGF-β1 gene.

The group of genetic polymorphisms relating to “a gene” is not limited to a polymorphism present in an exon or intron of the gene, but also includes a polymorphism present in, for example, a promoter region, 3′-noncoding region or 5′-noncoding region. In general, polymorphism in a coding region may vary the amino acid sequence or the expression level of mRNA. A polymorphism in a control element may vary the expression level of mRNA or splicing. Both polymorphisms may possibly vary the expression level or properties of a protein.

Concrete examples of the genetic polymorphisms belonging to any one of the groups a) to l) include, but are not limited to, the genetic polymorphisms shown in Tables 1-1 to 1-4. Of the groups a) to l), the group d) of genetic polymorphisms relating to nitrogen-monoxide synthase, group f) of genetic polymorphisms relating to IRS1 gene, group h) of genetic polymorphisms relating to the muscle glycogen synthase gene, and group i) of genetic polymorphisms relating to NADP-NADPH oxidase p22phox can be totally grouped as a group relating to the insulin resistance-vascular endothelial function. The group e) of genetic polymorphisms relating to TNF-α gene, group k) of genetic polymorphisms relating to heat shock protein 70-1, and group 1) of genetic polymorphisms relating to TGF-β1 gene can be totally grouped as a group relating to inflammatory response. The group a) of genetic polymorphisms relating to the renin-angiotensin system can be understood as a group relating to sympathetic nerve blood pressure. The group b) of genetic polymorphisms relating to the platelet function-coagulation system, and group j) of genetic polymorphisms relating to methylenetetrahydrofolate reductase can be totally grouped as a group relating to coagulation-fibrinolytic system. The group c) of genetic polymorphisms relating to lipid, and group g) of genetic polymorphisms relating to FABP2 gene can be totally grouped as a group relating to lipid. They can also be classified as follows.
(A) Insulin resistance-vascular endothelial function-related
(B) Inflammatory response
(C) Adhesion factor
(D) Sympathetic nerve-blood pressure
(E) Coagulation-fibrinolytic system
(F) Lipid

Genes constituting the renin-angiotensin system are observed in the blood vessel and cardiac muscle and are reported to play important roles in arteriosclerosis, cardiac hypertrophy and remodeling of the blood vessel and cardiac muscle. Accordingly, polymorphisms relating to the individual constitutional genes of the renin-angiotensin system are preferably used in the present invention.

Polymorphisms relating to genes associated with GP (glycoprotein) Ib, IX receptors and vWF (von Willebrand factor) on the platelet surface and genes relating to fibrinogen receptors GPIIb, IIIa on the platelet surface are suitably used in the present invention as polymorphisms belonging to the group of genetic polymorphisms relating to the platelet function-coagulation system. These genes are involved in the binding between the platelet and an injury site in the blood vessel in endothelial cell.

Regarding the coagulation factors, polymorphisms relating to genes relating to coagulation factor VII and fibrinogen β-chain are also suitably used in the present invention as polymorphisms belonging to the group of genetic polymorphisms relating to the platelet function-coagulation system. The gene relating to fibrinogen β-chain has been reported to have a significant relation with the serum fibrinogen level.

The group of genetic polymorphisms relating to nitrogen-monoxide synthase, the group of genetic polymorphisms relating to TNF-α gene, the group of genetic polymorphisms relating to IRS-1 gene, the group of genetic polymorphisms relating to FABP2 gene and the group of genetic polymorphisms relating to the muscle glycogen synthase gene are involved in insulin resistance.

MTHFR (methylenetetrahydrofolate reductase) is a metabolic enzyme for homocysteine. It has been reported that an increase in blood homocysteine level is an independent risk factor of cardiac blood vessel disease.

The plural genetic polymorphisms preferably includes at least two genetic polymorphisms belonging to different groups of the groups a) to l), for higher synergistic effect or additional effect of the combination against the risk and higher contribution to the risk. The plural genetic polymorphisms more preferably includes at least three genetic polymorphisms belonging to different groups of the groups a) to l).

The plural genetic polymorphisms specifically preferably includes at least one genetic polymorphism belonging to the group of genetic polymorphisms relating to the platelet function-coagulation system, and at least one genetic polymorphism belonging to at least one of the group of genetic polymorphisms relating to the renin-angiotensin system, the group of genetic polymorphisms relating to methylenetetrahydrofolate reductase (MTHFR) and the group of genetic polymorphisms relating to lipid, for higher contribution to the risk. The combination including at least the group of genetic polymorphisms relating to methylenetetrahydrofolate reductase (MTHFR) and the group of genetic polymorphisms relating to lipid is also preferred.

A genetic polymorphism relating to PAI-1 gene is suitably used as the genetic polymorphism belonging to the group of genetic polymorphisms relating to the platelet function-coagulation system. A genetic polymorphism relating to ACE gene is suitably used as the genetic polymorphism belonging to the group of genetic polymorphisms relating to the renin-angiotensin system. A genetic polymorphism relating to HUMPONA gene is suitably used as the genetic polymorphism belonging to the group of genetic polymorphisms relating to lipid.

The risk of arteriosclerosis inherent to a combination of plural genetic polymorphisms can be determined by analyzing the relation between the measured risk of arteriosclerosis in the population and the combination of plural genetic polymorphisms. Thus, the risk of arteriosclerosis inherent to the combination can be previously set based on the determined value.

The risk of arteriosclerosis can be appropriately selected according to the purpose from known factors of carotid arteriosclerosis which are determined, for example, by measuring the thickness of the carotid artery. The process for measuring the thickness of the carotid artery is not specifically limited, but is generally a process of measuring the carotid arterial intima-media thickness (IMT) using an ultrasonic tomography apparatus. This process is a non-invasive and quantitative measurement process for measuring the thickness of the carotid artery to which ultrasounds can arrive. The measurement of the carotid arterial intima-media thickness (IMT) has been taken as an example, but the measurement process for determining the risk of arteriosclerosis is not limited thereto.

The ultrasonic tomography apparatus preferably has a linear type pulse echo probe with a center frequency of 7.5 MHz or more. The carotid artery outside the crania lies in a shallow layer under the skin, and an apparatus with a frequency of 7.5 MHz or more can be used and can yield a high resolution (range resolution: 0.1 mm).

The blood vessel is analyzed in an echo image as a two-layer structure comprising an inner layer with a low echo intensity on the inner side of the blood vessel, and an outer layer with a high echo intensity. The invetors have verified, based on the observation of 104 healthy cases, that IMT substantially linearly increases with ageing from teens to seventies and does not exceed 1.1 mm. They have determined IMT of healthy subjects in terms of age according to the following equation.
IMT=0.08×Age+0.3 (3<Age<80 yr) [1]

The known indicators of carotid arteriosclerosis that can indicate the risks of arteriosclerosis include, for example, maximum IMT (Max-IMT) indicating the maximum of IMT, average IMT (AvgIMT) indicating the average of IMT, plaque score (PS) and carotid artery stiffness. A specific indicator has not yet been decided. Further, various measurement methods are used for the individual indicators.

For example, some investigators define MaxIMT as the maximal intima-media thickness in longitudinal sectional images in anterior oblique, side and posterior oblique positons and AvgIMT as the average of three points at proximal position of 1 cm and distal position of 1 cm with the site showing the MaxIMT as center; some other define AvgIMT as the maximum of a total of 12 values in thickness of the proximal wall (near wall) and distal wall (far wall) with respect to the skin in three longitudinal sections from the left and right common carotid arteries (common carotid: CC) to the carotid artery bifurcation and the internal carotid artery (internal carotid: IC); and some other define AvgIMT as the average of the thickness of the left and right carotid arteries. Further, some investigators deine mean IMT as an average thickness of a specific area in far wall, and some other define the thickness of the far wall at proximal position of 10 mm from the bifurcation of one carotid artery as the indicator.

The plaque score (PS) refers to the total thickness of plaques having IMT of 1.1 mm or more in individual sections in the left and right carotid arteries, in which each carotid artery is divided into 4 sections of 15 mm size with the bifurcation as the basis. In addition, the total of the number of plaques (IMT: 1.1 mm or more) in the sections 3 to 4 is defined as the plaque number (PN) and is used as an indicator.

The carotid artery stiffness is a numerical value determined from the diameters of the carotid artery in the systole and diastole.

The method using the thickness of the far wall at proximal position of 10 mm from the bifurcation of one carotid artery as the indicator employs a simple measurement procedure and is believed to have less measurement error, since the common carotid artery shows less lesion. IMT is an indicator indicating the maximal lesion of the carotid artery. PS can provide an overview of the carotid artery with advanced arteriosclerosis, but stands 0 in non-advanced cases (thickness: less than 1.1 mm). Thus, a suitable indicator varies depending on the subject to be measured and the disease. In cases with diabetes and/or hyperlipemia, the carotid artery wall often thickens relatively uniformly, and AvgIMT and mean IMT serve as important indicators. In cases with hypertension, plaque is often observed, and PS, PN and MaxIMT act as efficacious indicators.

The risk of arteriosclerosis inherent to the combination of plural genetic polymorphisms can be set using a variety of known indicators for carotid arteriosclerosis as described above. Preferred examples of the setting procedure are setting depending on whether or not the combination has a significant positive correlation with the carotid arterial intima-media thickness (for example 1 or 0); and setting depending on the odds ratio in which the combination has a significant positive correlation with the carotid arterial intima-media thickness. The setting depending on an increase in the carotid arterial intima-media thickness is also preferred. This setting can indicate a general risk of arteriosclerotic disease. The increment in average IMT (ΔIMT) and the increment in maximum IMT (ΔPIMT), for example, can be used as the increase in the carotid arterial intima-media thickness. ΔIMT is typically preferred as an indicator indicating the general risk of arteriosclerotic disease. Many reports have been made on the relation between the increase in the carotid arterial intima-media thickness and the arteriosclerotic disease. In particular, regarding ΔIMT, the odds of myocardial infarction increases 4.9 times per 0.339-mm increase of ΔIMT (Yamasaki. Diabetes Care 2000 (9)). Accordingly, an aspect using ΔIMT as the risk of arteriosclerosis can determine the risk of arteriosclerotic disease very efficaciously. As is described above, the increase in the carotid arterial intima-media thickness can be used as the risk of arteriosclerosis to indicate the risk of arteriosclerotic disease. Alternatively, the risk of arteriosclerosis can be calculated from the increase in the carotid arterial intima-media thickness using an appropriate function.

The increase in the carotid arterial intima-media thickness (e.g., ΔIMT and ΔPIMT) can be indicated by a partial regression coefficient calculated from IMT measurements or PIMT measurements measured in the population according to the multiple regression analysis method.

The risk evaluation process for evaluating the risk of arteriosclerosis caused by genetic polymorphisms may comprise plural processes. More specifically, the risk of arteriosclerosis can be decided on each of plural sets of the genetic polymorphisms, respectively.

When the risk evaluation process comprises a single process, the risk of arteriosclerosis decided by the process can be used as intact as the risk of arteriosclerotic disease. When the risk evaluation process comprises two or more processes, the risks of arteriosclerosis decided by the processes can be used as the risk of arteriosclerotic disease by synthetically subjecting them to linear operation.

The method for determining the risk of arteriosclerotic disease of the present invention may further comprise a risk evaluation process for evaluating the risk of arteriosclerosis caused by environmental factor from the information of the subject on the environmental factor based on the risk of arteriosclerosis inherent to the environmental factor.

Such environmental factors include, for example, age, sexuality, hypertension, diabetes, hyperlipemia, obesity, smoking, and hemoglobin Alc level, as reported.

Vitelli et al. have reported that an increase of 1% in hemoglobin Alc increases the risk of arteriosclerosis 1.77 times. This report is an estimated result of comparison of 208 non-diabetes subjects with carotid artery hyperplasia (average IMT: 1.21 mm) with 208 non-diabetes subjects without hyperplasia (average IMT: 0.63 mm) in an arteriosclerosis risk in communities study (ARIC Study) [Vitelli L L. Diabetes Care 1997; 20: 1454-8].

Smoking is believed to be a risk factor of arteriosclerosis. An arteriosclerosis risk in communities study (ARIC Study) indicates a strong correlation between smoking history and IMT and indicates that smoking acts as a further strong acceleration factor in diabetes or hypertension patients [Howard G, JAMA 1998; 279: 119-24.].

Sutton-Tyrrell et al. have reported that menopause accelerates arteriosclerosis of women. They have investigated on IMT and plaque lesions in premenopausal and postmenopausal women of similar age and found that the average IMT significantly increases from 0.69 mm to 0.77 mm and the ratio of women with plaque significantly increases from 25% to 54% after menopause [Sutton-Tyrrell K, Stroke 1998; 29: 1116-21].

Various infectious diseases are considered as a cause of arteriosclerosis. Nieto et al. indicates the possibility of cytomegalovirus as an advance factor of arteriosclerosis by extracting an IMT-advanced group and a non-advanced group and searching antibody titers of cytomegalovirus and found that the odds ratio for a group of cases with an antibody titer of 20 or more compared with a group of cases with an antibody titer less than 4 was significantly high of 5.3 in a in an atherosclerosis risk in communities study (ARIC Study) [Nieto F J, Circulation 1996; 94: 922-7].

The invetors have made a multiple regression analysis using IMT of subjects with type I diabetes, type II diabetes and borderline cases as dependent variables and have reported that the age, diabetes morbidity period and hemoglobin Alc level are independent risk factors in the subjects with type I diabetes (younger than 30-year-old); that the age, hemoglobin Alc level, non-HDL cholesterol, systolic blood pressure and smoking history are independent risk factors in patients with Type II diabetes (30-year-old or older); and that the systolic blood pressure and smoking, in addition to ageing are risk factors in borderline diabetes cases [Yamasaki Y, Diabetes 1994; 43: 634-639].

The age, sexuality, diabetes morbidity period and hemoglobin Alc level are important among the environmental factors. The increase in the carotid arterial intima-media thickness caused by these environmental factors can be used as the risk of arteriosclerosis.

The risk evaluation process for evaluating the risk of arteriosclerosis caused by environmental factor may comprise plural processes.

The method for determining the risk of arteriosclerotic disease of the present invention may further comprise a risk evaluation process for evaluating the risk of arteriosclerosis depending on the carotid arterial intima-media thickness from an actually measured carotid arterial intima-media thickness of the subject based on the risk of arteriosclerosis inherent to the carotid arterial intima-media thickness. The risk of arteriosclerosis inherent to the carotid arterial intima-media thickness can also be set, for example, at one time the carotid arterial intima-media thickness of the subject. When the carotid arterial intima-media thickness of the subject is directly measured, the risk of development or progress of arteriosclerotic disease at the time of measurement can be determined using the measurement value. In the present invention, risks such as risk of development in the future and tendency for the advance of the disease can be predicted, because risks caused by the combination of genetic polymorphisms are combined. In particular, predicting future risks in young subjects with not so advanced hyperplasia at the time of measurement allows one having a high risk to prevent the disease typically by improving the lifestyle to thereby prevent onset of arteriosclerotic disease.

When the method for determining the risk of arteriosclerotic disease comprises plural risk evaluation processes such as a risk evaluation process for evaluating the risk of arteriosclerosis caused by environmental factors and a risk evaluation process for evaluating the risk of arteriosclerosis depending on the carotid arterial intima-media thickness, the method may include a process for calculating the risk of arteriosclerotic by synthetically subjecting the risks of arteriosclerosis decided in the individual risk evaluation processes to linear operation.

According to the method for determining the risk of arteriosclerotic disease of the present invention, genetic polymorphisms are combined and grasped as risk factors which affect the risk of arteriosclerotic disease, and thereby the risk can be determined highly precisely. This is based on the findings of the present inventors that a combination of plural genetic polymorphisms affects the risk not only additionally but also synergistically, even when a single genetic polymorphism does not significantly affect the risk. The method for determining the risk of arteriosclerotic disease is a determination method which is realized by finely investigating the relation between the genetic polymorphisms and the carotid arterial intima-media thickness, an indicator that can be quantitatively measured both in healthy subjects and subjects with disease.

(Method for Measuring the Risk of Arteriosclerotic Disease)

The method for measuring the risk of arteriosclerotic disease of the present invention comprises a detection process for detecting the genotype of a subject on plural genetic polymorphisms, and a risk evaluation process for evaluating the risk of arteriosclerosis caused by genetic polymorphisms from the genotype of the subject on genetic polymorphisms decided in the detection process based on the risk of arteriosclerosis inherent to the combination of plural genetic polymorphisms. In the method, at least one combination of the plural genetic polymorphisms should have a significant relation with the carotid arterial intima-media thickness. Other configurations are not specifically limited.

The detection process can employ any procedure that detects the genotype of a subject on plural genetic polymorphisms. A specimen including DNA of the subject is used according to a general procedure. Examples of the specimen are blood, sputum, skin, bronchoalveolar washing, other body fluids, or tissues. As analytical procedure, many procedures are known, of which typical examples are as follows (Clin. Chem. 43: 1114-1120,1997). The sequence method directly sequences DNA regions including genetic polymorphisms. The PCR method specifically amplifies a certain genetic polymorphism alone using a primer specific to the genetic polymorphism. In the PCR method, nucleic acids regarding the genetic polymorphism is generally arranged farthest to the 3′-end. However, the design of the primer is not specifically limited, as long as the genetic polymorphism can be distinguished. The design includes in which region of the primer the genetic polymorphism is arranged and which other nucleotide sequences than the gene to be detected. For example, in the allele specific primer (ASP)-PCR method, the primer regarding genetic polymorphism is arranged next to the 3′-end. In the TaqMan method, an allele-specific probe is labeled with a fluorescent dye and a quencher at both ends and is hybridized with a target site, then a PCR is carried out by using a primer so designed as to amplify a region including the site. When the PCR from the primer reaches the region hybridized with the allele-specific probe, the fluorescent dye at the 5′-end of the hybridized probe is cleaved by the five prime nuclease activity of Taq polymerase and leaves the quencher to thereby emit fluorescence. This technique reveals how much the allele-specific probe is hybridized. The invader assay can determine which allele probe is hybridized in accordance with the same principle as the TaqMan method. This technique employs three oligonucleotides, i.e., an allele probe, an invader probe and a FRET probe. The allele probe comprises a specific sequence at 5′ side and a flap sequence at 3′ side from a template genetic polymorphism site. The invader probe comprises a specific sequence at 3′ side from a template genetic polymorphism site. The FRET probe includes a sequence complementary to the flap sequence. The MALDI-TOF/MS method identifies a genetic polymorphism in the following manner. A neighboring primer is prepared in a genetic polymorphism site, this region is amplified, only one base in the genetic polymorphism site is then amplified using ddNTP, and the type of the added ddNTP is identified using MALDI-TOF/MS. In the DNA chip method such as Hybrigene method, an oligonucleotide probe including genetic polymorphism is arranged on an array, and hybridization with a sample DNA which has been subjected to PCR amplification is detected. In addition, the molecular beacon method and ligation method are exemplified as known methods.

The risk evaluation process can employ processes similar to the processes described in the method for determining the risk of arteriosclerotic disease.

(Method for Revealing an Arteriosclerotic Disease-Associated Factor)

The method for revealing an arteriosclerotic disease-associated factor of the present invention comprises a process for revealing a set of arteriosclerosis-associated genetic polymorphisms from genetic polymorphisms of a subject by selectively uncovering the genotype of plural genetic polymorphisms relating to the set of arteriosclerosis-associated genetic polymorphisms among genetic polymorphisms of the subject,
in which the set of arteriosclerosis-associated genetic polymorphisms is a combination of plural genetic polymorphisms having a significant positive correlation with the carotid arterial intima-media thickness and is an arteriosclerotic disease-associated factor. Other configurations are not specifically limited. Even if the method further comprises any additional processes, it is within the scope of the present invention as long as it comprises the revealing process.

The human genome includes a great many genetic polymorphisms. When only one of them is employed, the odds ratio is low and incidence is limited. This disables the risk of arteriosclerosis to be predicted. Accordingly, if such genetic polymorphisms are grasped separately, factors relating to arteriosclerosis present as a combination in genetic polymorphisms of an individual cannot be found. According to the present invention, a multiplicity of populations have been analyzed to find that there are combinations of plural genetic polymorphisms having a significant positive correlation with the carotid arterial intima-media thickness. Based on these findings, these sets of arteriosclerosis-associated genetic polymorphisms are grasped as arteriosclerotic disease-associated factors. Genetic polymorphisms relating to these specific combinations of a tested specimen are selectively uncovered and are dealt as a whole. Thus, the arteriosclerotic disease-associated factors have been revealed for the first time.

The revealed arteriosclerotic disease-associated factor is very highly valuable as information for the determination of the risk of arteriosclerotic disease.

The “selectively uncover” means that a specific one is selected from a great many combinations of genetic polymorphisms and is uncovered.

The revealing process may include not only uncovering a set of genotypes relating to the combination of plural genetic polymorphisms as a whole, but also showing whether or not the selectively uncovered genotype of plural genetic polymorphisms corresponds to the set of arteriosclerosis-associated genetic polymorphisms (for example, represented by 0 or 1). It may also include showing the odds ratio of having a significant positive correlation with the carotid arterial intima-media thickness which is inherent to the set of arteriosclerosis-associated genetic polymorphisms, when the selectively uncovered genotype of plural genetic polymorphisms corresponds to the set of arteriosclerosis-associated genetic polymorphisms; and showing an increase in the carotid arterial intima-media thickness inherent to the set of arteriosclerosis-associated genetic polymorphisms, when the selectively uncovered genotype of plural genetic polymorphisms corresponds to the set of arteriosclerosis-associated genetic polymorphisms. The method is not specifically limited, as long as it can reveal the set of arteriosclerosis-associated genetic polymorphisms from among genetic polymorphisms of the subject.

The set of arteriosclerosis-associated genetic polymorphisms preferably includes at least one set of arteriosclerosis-associated genetic polymorphisms selected from the sets of arteriosclerosis-associated genetic polymorphisms shown in Tables 1-1 to 1-4 and Tables 9-1 to 9-2.

The set of arteriosclerosis-associated genetic polymorphisms is typically preferably selected so that, when cases having a carotid arterial intima-media thickness at least 0.2 mm larger than the average of carotid arterial intima-media thickness of healthy subjects are defined as arteriosclerotic disease cases and the other cases are defined as non-arteriosclerotic disease cases, cases having at least one set of arteriosclerosis-associated genetic polymorphisms occupy 30% or more of a population of arteriosclerotic disease cases comprising at least 150 cases, and cases having at least one set of arteriosclerosis-associated genetic polymorphisms occupy 15% or less of a population of non-arteriosclerotic disease cases comprising at least 150 cases.

The set of arteriosclerosis-associated genetic polymorphisms preferably comprises two to five genetic polymorphisms and may comprise at least three genetic polymorphisms.

The set of arteriosclerosis-associated genetic polymorphisms also preferably include at least two genetic polymorphisms belonging to any of following groups a) to l):
a) group of genetic polymorphisms relating to the renin-angiotensin system;

b) group of genetic polymorphisms relating to the platelet function-coagulation system;
c) group of genetic polymorphisms relating to lipid;
d) group of genetic polymorphisms relating to nitrogen-monoxide synthase;
e) group of genetic polymorphisms relating to TNF-α gene;
f) group of genetic polymorphisms relating to IRS-1 gene;
g) group of genetic polymorphisms relating to FABP2 gene;
h) group of genetic polymorphisms relating to the muscle glycogen synthase gene;
i) group of genetic polymorphisms relating to NADP-NADPH oxidase p22phox;
j) group of genetic polymorphisms relating to methylenetetrahydrofolate reductase;
k) group of genetic polymorphisms relating to heat shock protein 70-1; and
l) group of genetic polymorphisms relating to TGF-β1 gene.

The method may further include a detection process for detecting the genotype of the subject on the plural genetic polymorphisms before the revealing process.

(Method for Detecting Genetic Polymorphisms)

The method for detecting genetic polymorphisms of the present invention comprises a process for detecting the genotype of a subject on genetic polymorphisms constituting at least one set of arteriosclerosis-associated genetic polymorphisms selected from the sets of arteriosclerosis-associated genetic polymorphisms shown in Tables 1-1 to 1-4 and Tables 9-1 to 9-2, in which the detection result is used for the determination of the risk of arteriosclerotic disease. Other configurations are not specifically limited.

(Genetic Marker)

The genetic marker of the present invention includes genetic polymorphisms constituting at least one set of arteriosclerosis-associated genetic polymorphisms selected from the sets of arteriosclerosis-associated genetic polymorphisms shown in Tables 1-1 to 1-4 and Tables 9-1 to 9-2. Other configurations are not specifically limited. The gene marker is an aspect in which the combination of genetic polymorphisms is used as markers for arteriosclerotic disease.

(Kit for Analyzing Genetic Polymorphisms)

The kit for analyzing genetic polymorphisms of the present invention comprises a pair of primers capable of specifically amplifying genes constituting at least one set of arteriosclerosis-associated genetic polymorphisms selected from the sets of arteriosclerosis-associated genetic polymorphisms shown in Tables 1-1 to 1-4 or nucleic acid probe capable of specifically hybridizing the gene, wherein the kit is so configured as to detect at least one of the sets of arteriosclerosis-associated genetic polymorphisms shown in Tables 1-1 to 1-4. The kit is not specifically limited as long as it has the above configuration.

The kit must have any of primers and probes for detecting at least two genetic polymorphisms selected from the genetic polymorphisms relating to the set of genetic polymorphisms shown in (Tables 2-1 and 2-2). This enables the kit to detect at least one set of arteriosclerosis-associated genetic polymorphisms selected from the sets of arteriosclerosis-associated genetic polymorphisms shown in Tables 1-1 to 1-4. The kit for analyzing genetic polymorphisms of the present invention can be any kit as long as it can analyze the plural genetic polymorphisms. For example, the kit may comprise a primer for detecting one genetic polymorphism and a probe for detecting another genetic polymorphism. The individual genetic polymorphisms shown in Tables 2-1 and 2-2 can be detected by any of the methods or procedures described in the genetic polymorphisms detection process. Among them, the hybrigene method using PCR, TaqMan method, invader method and ASP-PCR method using a nucleic acid probe capable of specifically hybridizing with a gene having genetic polymorphism are suitably used. Accordingly, the kit for analyzing genetic polymorphisms must include at least one of primers and probes for use in the process for detecting these genetic polymorphisms. In the PCR method for detecting genetic polymorphisms, nucleic acids regarding the genetic polymorphism is generally arranged farthest to the 3′-end. However, the design of the primer is not specifically limited, as long as the genetic polymorphisms can be distinguished. The design includes in which region of the primer the genetic polymorphism is arranged and which other nucleotide sequences than the gene to be detected. For example, in the Allele Specific Primer (ASP)-PCR method, the primer regarding genetic polymorphism is arranged next to the 3′-end. Likewise, the probe can be designed in any way regarding its sequence, as long as the genetic polymorphisms can be distinguished.

The set of arteriosclerosis-associated genetic polymorphisms to be detected by the kit for analyzing genetic polymorphisms can be any set shown in Tables 1-1 to 1-4. Among them, a group including three or more genetic polymorphisms is preferred for higher sensitivity for detecting the risk.

For predicting the risk, the kit more preferably comprises a primer or probe for detecting at least two genetic polymorphisms selected from the genetic polymorphisms shown in Tables 2-1 and 2-2 and detects such a set of arteriosclerosis-associated genetic polymorphisms that, when cases having a carotid arterial intima-media thickness at least 0.2 mm larger than the average of carotid arterial intima-media thickness of healthy subjects are defined as arteriosclerotic disease cases and the other cases are defined as non-arteriosclerotic disease cases, cases having a combination of genotypes having a significant positive correlation with the carotid arterial intima-media thickness in the sets of arteriosclerosis-associated genetic polymorphisms shown in Tables 1-1 to 1-4 occupy 30% or more of a population of arteriosclerotic disease cases comprising at least 150 cases, and occupy 15% or less of a population of non-arteriosclerotic disease cases comprising at least 150 cases, where the selected genetic polymorphisms can constitute the sets of arteriosclerosis-associated genetic polymorphisms.

Another aspect of the kit for analyzing genetic polymorphisms of the present invention comprises a pair of primers capable of specifically amplifying genes constituting at least one set of arteriosclerosis-associated genetic polymorphisms selected from the sets of arteriosclerosis-associated genetic polymorphisms shown in Tables 9-1 to 9-2 or nucleic acid probe capable of specifically hybridizing the gene, wherein the kit is so configured as to detect at least one of the sets of arteriosclerosis-associated genetic polymorphisms shown in Tables 9-1 to 9-2. The kit is not specifically limited as long as it has the above configuration.

For predicting the risk, the kit also preferably comprises a primer or probe for detecting at least two genetic polymorphisms selected from the genetic polymorphisms shown in Tables 10-1 and 10-2, and is so configured that,
when cases having a carotid arterial intima-media thickness at least 0.2 mm larger than the average of carotid arterial intima-media thickness of healthy subjects are defined as arteriosclerotic disease cases and the other cases are defined as non-arteriosclerotic disease cases, cases having at least one combination of genotypes having a significant positive correlation with the carotid arterial intima-media thickness in the sets of arteriosclerosis-associated genetic polymorphisms shown in Tables 1-1 to 1-4 occupy 70% or more of a population of arteriosclerotic disease cases comprising at least 150 cases, and occupy 35% or less of a population of non-arteriosclerotic disease cases comprising at least 150 cases, in which the selected genetic polymorphisms can constitute the sets of arteriosclerosis-associated genetic polymorphisms.

The kit also preferably comprises plural primers and/or probes for detecting genetic polymorphisms belonging to different groups of following groups a) to l):
a) group of genetic polymorphisms relating to the renin-angiotensin system;
b) group of genetic polymorphisms relating to the platelet function-coagulation system;
c) group of genetic polymorphisms relating to lipid;
d) group of genetic polymorphisms relating to nitrogen-monoxide synthase;
e) group of genetic polymorphisms relating to TNF-α gene;
f) group of genetic polymorphisms relating to IRS1 gene;
g) group of genetic polymorphisms relating to FABP2 gene;
h) group of genetic polymorphisms relating to the muscle glycogen synthase gene;
i) group of genetic polymorphisms relating to NADP-NADPH oxidase p22phox;
j) group of genetic polymorphisms relating to methylenetetrahydrofolate reductase;
k) group of genetic polymorphisms relating to heat shock protein 70-1; and
l) group of genetic polymorphisms relating to TGF-β1 gene.
   (Array for Determining the Risk of Arteriosclerotic Disease)

The array for determining the risk of arteriosclerotic disease of the present invention comprises a probe for detecting genetic polymorphisms constituting at least one set of arteriosclerosis-associated genetic polymorphisms selected from the sets of arteriosclerosis-associated genetic polymorphisms shown in Tables 1-1 to 1-4 and Tables 9-1 to 9-2. Other configurations are not specifically limited, and the array can comprise any material appropriately selected from known materials as long as not adversely affecting the advantages of the present invention.

The array for determining the risk of arteriosclerotic disease can be used in any technique such as a technique of immobilizing a prepared probe to a substrate or a technique of Affimetrix of synthesizing the array on a substrate. The substrate to which the probe is immobilized is not specifically limited and may comprise a known material such as glass plate or filter. The length of the probe and the type of nucleic acids to be used are not specifically limited, as long as genetic polymorphisms can be detected. For better sensitivity, the region including genetic polymorphisms is preferably amplified by PCR previously. In particular, a technique of amplifying the region including genetic polymorphisms using a labeled primer is preferably used for better sensitivity and convenience. In the hybrigene method, for example, the region including genetic polymorphisms is amplified using a primer labeled with biotin, this is added to the array for hybridization, and nucleic acids not hybridized are removed by rinsing. Then, a hybridized probe is detected using an avidin-labeled fluorescent dye. This technique can detect the genetic polymorphisms with high sensitivity.

The array for determining the risk of arteriosclerotic disease preferably comprises probes for detecting genetic polymorphisms belonging to different ones of following groups a) to l):
a) group of genetic polymorphisms relating to the renin-angiotensin system;
b) group of genetic polymorphisms relating to the platelet function-coagulation system;
c) group of genetic polymorphisms relating to lipid;
d) group of genetic polymorphisms relating to nitrogen-monoxide synthase;
e) group of genetic polymorphisms relating to TNF-α gene;
f) group of genetic polymorphisms relating to IRS-1 gene;
g) group of genetic polymorphisms relating to FABP2 gene;
h) group of genetic polymorphisms relating to the muscle glycogen synthase gene;
i) group of genetic polymorphisms relating to NADP-NADPH oxidase p22phox;
j) methylenetetrahydrofolate reductase (MTHFR)-related genetic polymorphisms group;
k) group of genetic polymorphisms relating to heat shock protein 70-1; and
l) group of genetic polymorphisms relating to TGF-β1 gene.
   (Apparatus for Determining the Risk of Arteriosclerotic Disease)

The apparatus for determining the risk of arteriosclerotic disease of the present invention uses a computer and comprises a data table on the risk of arteriosclerosis in which combinations of plural genetic polymorphisms are listed with corresponding risks of arteriosclerosis, and detection means for checking inputted combinations of plural genetic polymorphisms of a subject against the combinations of plural genetic polymorphisms in the data table on the risk of arteriosclerosis and, when there is a combination of genetic polymorphisms matching between the two, detecting the risk of arteriosclerosis corresponding to the combination of genetic polymorphisms. Other configurations are not specifically limited. The detected risk of carotid artery can be used as intact as the risk of arteriosclerotic disease or can be appropriately converted into, for example, a simple numerical value for use as the risk of arteriosclerotic disease.

The plural genetic polymorphisms in the data table on the risk of arteriosclerosis preferably include at least one combination of genetic polymorphisms having a significant relation with the carotid arterial intima-media thickness.

The data table on the risk of arteriosclerosis may coordinate, as the risk of arteriosclerosis, 1 unit with a combination of plural genetic polymorphisms having a significant positive correlation with the carotid arterial intima-media thickness.

Further, the data table on the risk of arteriosclerosis may coordinate, as the risk of arteriosclerosis, an odds ratio for the carotid arterial media-media thickness of exceeding a normal range with a combination of plural genetic polymorphisms having a significant positive correlation with the carotid arterial intima-media thickness.

A combination of genetic polymorphisms is preferably defined to have a significant positive correlation with the carotid arterial intima-media thickness, for example, in the case where the odds ratio for the carotid arterial media-media thickness of exceeding a normal range stands at a specific level or more, or in the case where the average of the carotid arterial intima-media thickness shows a significant difference.

The apparatus for determining the risk of arteriosclerotic disease of the present invention may further comprise:
a data table on the risk of arteriosclerosis in which the presence or absence of, or the numerical value of an environmental factor is listed corresponding to a risk of arteriosclerosis, and
detection means for checking inputted presence or absence of, or numerical value of an environmental factor of a subject against the presence or absence of, or the numerical value of an environmental factor in the data table on the risk of arteriosclerosis and detecting a risk of arteriosclerosis corresponding to the presence or absence of, or the numerical value of an environmental factor. This allows the apparatus to determine the risk of arteriosclerotic disease further precisely by inputting information on environmental factors which act as risk factors.

When the apparatus for determining the risk of arteriosclerotic disease comprises a plurality of detection means, the risk of arteriosclerotic disease can also be determined based on the additional value of plural risks of carotid artery detected by the plural detection means.

The apparatus for determining the risk of arteriosclerotic disease of the present invention may further comprise:
a data table on the risk of arteriosclerosis in which carotid arterial intima-media thickness is listed corresponding to a risk of arteriosclerosis, and
detection means for checking an inputted carotid arterial intima-media thickness of a subject against the carotid arterial intima-media thickness in the data table on the risk of arteriosclerosis and detecting a risk of arteriosclerosis corresponding to the carotid arterial intima-media thickness.

The apparatus for determining the risk of arteriosclerotic disease of the present invention may be so configured that, when the carotid artery risk detected by the detection means and an additional value of carotid artery risks extracted by the plural extraction means is expressed as an increment in the carotid arterial intima-media thickness, the risk of arteriosclerotic disease may be determined based on the additional value thereof, using an inputted carotid arterial intima-media thickness of the subject as intact.

The apparatus for determining the risk of arteriosclerotic disease of the present invention may further comprise determination means for determining the risk of arteriosclerotic disease based on an additional value of an inputted carotid arterial intima-media thickness of a subject with one of the carotid artery risk detected by the detection means and an additional value of carotid artery risks extracted by the plural extraction means. The vascular coat pressure measuring means may be a vascular coat pressure measuring device other than a computer, or a device including a vascular coat pressure measuring device and an analytical computer in combination. The vascular coat pressure measuring device can be appropriately selected from known vascular coat pressure measuring devices according to the purpose. When the vascular coat pressure measuring means includes a computer, the computer of the vascular coat pressure measuring means may be integrated with the computer for storing the data table s and detecting the risk.

(A Recording Medium for Storing the Program for Determining the Risk of Arteriosclerotic Disease)

The recording medium of the present invention comprises a program for determining the risk of arteriosclerotic disease, wherein the program comprises the steps of checking inputted combinations of plural genetic polymorphisms of a subject against the combinations of plural genetic polymorphisms in a data table on the risk of arteriosclerosis in which combinations of plural genetic polymorphisms are listed with corresponding risks of arteriosclerosis, the data table being recorded in a computer, and detecting the risk of arteriosclerosis corresponding to the combination of genetic polymorphisms when there is a combination of genetic polymorphisms matching between the two as a result of checking.

The data table on the risk of arteriosclerosis in which combinations of plural genetic polymorphisms are listed with corresponding risks of arteriosclerosis may be one having the same functions as described in the apparatus for determining the risk of arteriosclerotic disease.

The program for determining the risk of arteriosclerotic disease is preferably so configured that, when cases having a carotid arterial intima-media thickness at least 0.2 mm larger than the average of carotid arterial intima-media thickness of healthy subjects are defined as arteriosclerotic disease cases and the other cases are defined as non-arteriosclerotic disease cases, cases having at least 1 unit of the risk of arteriosclerosis occupy 30% or more, more preferably 50% or more, and further preferably 60% or more, of a population of arteriosclerotic disease cases comprising at least 150 cases, and cases having at least 1 unit of the risk of arteriosclerosis occupy 15% or less of a population of non-arteriosclerotic disease cases comprising at least 150 cases. This program is very useful from the viewpoint of risk determination.

### EXAMPLES

The present invention will be further illustrated with reference to several examples below, which are not intended to limit the scope of the present invention.

### Example 1

Relation between Genetic Polymorphisms and IMT

The carotid arterial intima-media thickness of 200 healthy subjects and 200 patients with Type II diabetes was measured to give an average IMT and a peak IMT (PIMT) on each of the subjects. Separately, DNAs were extracted from the blood sampled from the healthy subjects and patients with diabetes by the phenol-chloroform method. DNAs including genetic polymorphisms of ACE, AGT, SERPINE1, APOE, APOB, PON1, LOC113690, MTHFR, IRS1 and FABP2 shown in Table 3 were amplified using the obtained DNAs as a template, according to a known ASP-PCR method using genetic polymorphism-specific primers. The individual genetic polymorphisms were determined by checking the presence or absence of PCR products by means of agarose electrophoresis.

The carotid arterial intima-media thickness was measured in the following manner. The thickness at three or more points was successively measured from two directions with a high-resolution ultrasonic tomography apparatus. The average of measurement values of one subject was defined as the average IMT and the maximum thereof was defined as the peak IMP. ΔIMT and ΔPIMT inherent to a genotype having risk factors homozygously were calculated on the single genetic polymorphism from the data according to the linear multiple regression analysis procedure. ΔIMT herein represents an increment in the average carotid arterial intima-media thickness, and ΔPIMT represents an increment in the maximum carotid arterial intima-media thickness. The results are shown in Table 5.

TABLE 5

| | Associated | | Racial | | |
|---|---|---|---|---|---|
| No. | factor | Symbol | difference | ΔPIMT | ΔIMT |
| | Genetic polymorphisms relating to the renin-angiotensin system | | | | |
| 4 | ACE | ACE | | 0.54 | 0.08 |
| 5 | angioten- | AGT | | 0.12 | 0.094 |
| | sinogen | | | | |
| | Genetic polymorphisms relating to platelet | | | | |
| | function-coagulation system | | | | |
| 15 | PAI-1 | SERPINE1 | | 0.3 | 0.12 |
| | Genetic polymorphisms relating to lipid | | | | |
| 16 | apopro- | APOE | | 0.5 | 0.13 |
| | tein E | | | | |
| 21 | apopro- | APOB | | 0.52 | 0.13 |
| | tein B | | | | |
| 24 | HUMPONA | PON1 | | 0.179 | 0.108 |
| | Other factors | | | | |
| 29 | p22phox | LOC113690 | | | * 0.15 |
| 30 | MTHFR | MTHFR | Japanese = | 0.49 | * 0.29 |
| | | | Westerner | | |
| 33 | IRS-1 | IRS1 | Westerner >> | 0.36 | 0.07 |
| | | | Japanese | | |
| 35 | fatty acid | FABP2 | | 0.48 | 0.08 |
| | binding | | | | |
| | protein | | | | |

ΔPIMT and ΔIMT are expressed in the unit millimeter (mm).

In the table, “*” represents a significant relation (P<0.05) with ΔIMT. “Number” represents the genetic polymorphism with the same number in Tables 3 and 4.

These results show that each of MTHFR and p22phox genetic polymorphisms respectively has a significant relation with ΔIMT increase.

Next, ΔIMT inherent to a combination of genotypes having risk factors homozygously was calculated on two genetic polymorphisms of PON1 and MTHFR or of PON1 and SERPINE1. ΔIMT was *0.301 mm in the case with a genotype having the risk factors relating to PON1 and MTHFR homozygously. ΔIMT was 0.16 mm in the case with a genotype having the risk factors relating to PON1 and SERPINE1 homozygously.

These results show that when plural genetic polymorphisms, such as PON1 and MTHFR, relating to different functions are combined, the combination further affects ΔIMT more than a single genetic polymorphism, and that the risk can be set more precisely based on this. The combination of PON1 and SERPINE1 works to increase ΔIMT more than the single genetic polymorphism, but does not increase to such an extent as to yield a significant difference. It is considered that this combination does not so increase ΔIMT as compared with, for example, the combination shown in following Example 2, because these genetic polymorphisms roughly belong to the same group tightly relating to arteriosclerosis while they belong to different groups in Table 3.

### Example 2

Relation Between Combination of Three Genetic Polymorphisms and IMT, and Method for Determining the Risk of Arteriosclerotic Disease

Next, three genetic polymorphisms were detected, IMTs were measured, and ΔIMT inherent to the combination of the three genetic polymorphisms by the multiple regression analysis in 200 healthy subjects and 200 patients with Type II diabetes.

MTHFR genetic polymorphism with MTHFR as an associated factor, ACE genetic polymorphism with ACE as an associated factor and SERPINE1 genetic polymorphism with PAI-1 as an associated factor were selected as the combination of plural genetic polymorphisms. In each of the genetic polymorphisms, genotypes were classified as genotype (+) including the risk factors homozygously and as genotype (−) other than the above. The groups in the three different genotype polymorphism were combined and ΔIMT inherent to the combination of genetic polymorphisms was calculated and defined as the risk of arteriosclerosis. The results are shown in Table 6.

TABLE 6

| Genotype of genetic polymorphisms | | | | | |
|---|---|---|---|---|---|
| (A genotype having the risk factors | | | | | |
| homozygously is indicated as +, and | | | | | |
| the other genotypes are indicated as −) | | | | | |
| MTHFR | ACE | SERPINE1 | ΔPIMT | ΔIMT | p Value |
| + | − | − | not | 0.089 | 0.617 |
| | | | measured | | |
| − | + | − | not | −0.018 | 0.94 |
| | | | measured | | |
| − | − | + | not | 0.145 | 0.179 |
| | | | measured | | |
| + | + | − | 0.69 | 0.451 | 0.125 |
| + | − | + | 0.34 | 0.318 | *0.0135 |
| − | + | + | 0.22 | 0.127 | 0.3319 |
| + | + | + | not | 0.771 | **0.00007 |
| | | | measured | | |

Specifically, the risk of arteriosclerosis inherent to the combination of genotypes including the risk factors on all of the polymorphisms MTHFR, ACE and SERPINE1 homozygously was set at 0.771. The risk of arteriosclerosis inherent to the combination of genotypes including the risk factors on MTHFR and ACE polymorphisms homozygously but the risk factors on SERPINE1 polymorphism not homozygously was set at 0.451. Likewise, the risk of arteriosclerosis inherent to the combination of genotypes including the risk factors on MTHFR and SERPINE1 polymorphisms homozygously but the risk factors on ACE polymorphism not homozygously was set at 0.318. Likewise, the risk of arteriosclerosis inherent to the combination of genotypes including the risk factors on ACE and SERPINE1 polymorphisms homozygously but the risk factors on MTHFR polymorphism not homozygously was set at 0.127. Further, the risks of arteriosclerosis inherent to the combination of genotypes including the risk factors on one of MTHFR, ACE and SERPINE1 homozygously were set at 0.089, −0.018 and 0.145, respectively. The risk of arteriosclerosis inherent to the combination of genotypes including the risk factors on all of the three polymorphisms not homozygously was set at 0. The genotype of a subject on ACE, SERPINE1 and MTHFR polymorphisms was adopted to the predetermined risks of arteriosclerosis, and the risk of arteriosclerosis of the subject was decided. Specifically, if the subject has a genotype on the three polymorphisms including the risk factors homozygously, the risk of arteriosclerosis of the subject is decided to be 0.771. When the risk evaluation process is a single process, this value can be used as intact as the risk of arteriosclerotic disease of the subject.

These results show that the method for determining the risk of arteriosclerotic disease of the present invention, for example, can previously set these ΔIMT values as the risk of arteriosclerosis inherent to the combination of plural genetic polymorphisms, decide the risk of arteriosclerosis of the subject from the genotype of the plural genetic polymorphisms and determine the risk of arteriosclerotic disease highly precisely.

### Example 3

Relation Between Risk Factors Other than Genetic Polymorphisms and IMT

The relation of each of age, sexuality, diabetes morbidity period and hemoglobin Alc level with ΔIMT was analyzed, respectively, in 200 healthy subjects and 200 patients with Type II diabetes by the multiple regression analysis. The determined partial regression coefficients are shown below.

| Environmental factor | Partial regression coefficient | |
|---|---|---|
| Age | +0.015 | mm/yr |
| Male | +0.178 | mm |
| Diabetes morbidity period | +0.006 | mm/yr |
| Hemoglobin Alc level | +0.024 | mm/% |

These environmental factors act as risk factors independently. The method for determining the risk of arteriosclerotic disease of the present invention may further include a risk evaluation process on these factors.

For example, when the method includes a risk evaluation process relating to the age and sexuality among the environmental factors, and a subject to be determined is 30 years old, the risk of arteriosclerosis inherent to the age is set at “Age”×0.015 and the risk of carotid artery arteriosclerosis of the subject inherent to the age is 0.45. If the subject is a male, the risk of arteriosclerosis inherent to the sexuality among the environmental factors is 0.178 (0 in the case of a female).

If the subject has a genotype including the risk factors on MTHFR and SERPINE1 polymorphisms homozygously and including the risk factors on ACE not homozygously among the three genetic polymorphisms in Example 2, the risk of arteriosclerosis inherent to the combination was set at 0.318 (Table 6), and the risk of arteriosclerotic disease of the subject is determined according to the following equation.
0.318+0.45+0.178=0.946

### Example 4

Next, further investigations were made in a larger population based on the above results on various combinations of a wide variety of genetic polymorphisms which are speculated to have a relation with, for example, arteriosclerosis. Sets of arteriosclerosis-associated genetic polymorphisms were decided and whether or not the disease onset can be predicted was examined.

A total of 49 genetic polymorphisms and 47 genes shown in Tables 2-1 and 2-2 were finally investigated. Among 57 genetic polymorphisms initially investigated, those with a polymorphism incidence of 1% or less were excluded.

These genetic polymorphisms were classified under individual arteriosclerosis onset factors, respectively, as following Groups (A) to (G) and were classified as shown in Tables 7-1 and 7-2. The numbers thereof are as follows.

Insulin resistance-vascular endothelial function-related Group (A): 5

| | | |
|---|---|---|
| Inflammatory response | Group (B): | 9 |
| Adhesion factor | Group (C): | 4 |
| Sympathetic nerve-blood pressure | Group (D): | 8 |
| Coagulation-fibrinolytic system | Group (E): | 11 |
| Lipid | Group (F): | 8 |
| Others | Group (G): | 4 |

TABLE 7-1

| Polymorphism | | Name of Genetic Polymorphism | | | Reference | |
|---|---|---|---|---|---|---|
| No. | Symbol | Name of Gene | site | ref. SNP ID | Number | Group |
| 1 | N1 | e NOS | T-786C + 4repeat | rs2070744 | 1 | A |
| 2 | NOS3 | e NOS | G894T(Glu298Asp) | rs1799983 | | A |
| 3 | IRS1 | IRS-1 | G3494A(Gly971Arg) | rs1801278 | | A |
| 4 | GSY | glycogen synthase | A/G(M416V) | rs5447 | 2 | A |
| 5 | | p22phox | C242T | | 3 | A |
| 6 | IL62 | Interleukin-6 | C-634G | rs1800796 | | B |
| 7 | N10 | TGF -beta 1 | T29C(Leu10Pro) | 329th of | 4 | B |
| | | | | AY330201 | | |
| 8 | TNFa2 | Tumor necrosis factor- α | G-308A | rs1800629 | 25 | B |
| 9 | MMP9 | MMP-9 = gelatinase B | C-1562T | rs3918242 | 5 | B |
| 10 | MMP12 | MMP-12 | A-82G | rs2276109 | 6 | B |
| 11 | MCP1 | MCP-1(chemokine) | A-2518G | rs1024611 | 7 | B |
| 12 | MPO | myeloperoxidase | G-463A | rs2333227 | 8 | B |
| 13 | FR1 | Flactalkine receptor | G84635A(Val249Ile) | rs3732379 | | B |
| | | CX3CR1 | | | | |
| 14 | CRP1 | C-reactive protein | G1059C | rs1800947 | 26 | B |
| 15 | CX37 | connexin37(gap junction | C1019T(Pro319Ser) | rs1764391 | 21 | C |
| | | protein) | | | | |
| 16 | ESL2 | E-selectin | A561C(Ser128Arg) | rs5361 | 9 | C |
| 17 | ICAM1 | intercellular adhesin | G/A(E469K) | rs5498 | | C |
| | | molecule 1 | | | | |
| 18 | ESL1 | E-selectin | G98T | rs1805193 | 22 | C |
| 19 | DRD2 | Dopamine D2 receptor | C/G(Ser311Cys) | rs1801028 | | D |
| 20 | ACE ID | ACE | I/D type | 1451 to 1738th | 23 | D |
| | | | | deletion of | | |
| | | | | X62855 | | |
| 21 | AGTR1-3 | AT1 receptor | A1166C | rs5186 | | D |
| 22 | AGT1 | Angiotensinogen | T704C(M235T) | JST050962, | 10 | D |
| | | | | rs699 | | |
| 23 | 3AR | beta 3 adreno receptor | T/C(Trp64Arg) | rs4994 | 27 | D |
| 24 | β 2AR4 | β 2-Adrenergic Receptor | C79G | rs1042714 | | D |
| 25 | β 2AR-1 | β 2-Adrenergic Receptor | A46G(Arg16Gly) | rs1042713 | | D |
| 26 | HANP1 | Human Atrial Natriuuretic | T2238C | rs5065 | | D |
| | | Peptide | | | | |
| 27 | GP3A | GP IIB IIIa | C1565T (PIA2) | rs5918 | 24 | E |
| 28 | G1A3 | GP Ia | A1648G | rs1801106 | 11 | E |
| 29 | GP1ba | Human Platelet Antigen-2 | C1018T(Thr145Met) | rs6065 | 28 | E |
| 30 | GP61 | Glycoprotein VI | T/C(Ser219Pro) | rs1613662 | | E |

TABLE 7-2

| Polymorphism | | Name of Genetic Polymorphism | | | Reference | |
|---|---|---|---|---|---|---|
| No. | Symbol | Name of Gene | site | ref. SNP ID | Number | Group |
| 31 | | Glycoprotien I a | C807T | | 12 | E |
| 32 | Tbm3 | thrombomodulin | G33A | 1487th of | 13 | E |
| | | | | M74564 | | |
| 33 | | Factor XII | C46T | rs1801020 | | E |
| 34 | FGA1 | alpha fibrinogen | A/G(Thr312Ala) | rs6050 | 29 | E |
| 35 | FGB3 | beta Fibrinogen | C148T | rs1800787 | 14 | E |
| 36 | PAI | PAI-I | 4G/5G at -668 | rs1799889 | 15 | E |
| 37 | MTHFR | MTHFR | C677T | rs1801133 | | E |
| 38 | ABCA1 | ABCA 1 | G1051A(Arg219Lys) | rs2230806 | 16 | F |
| 39 | PONA2 | HUMPONA | A172T(Met 55Leu) | rs3202100 | | F |
| 40 | PONA1 | PON1 | A584G(Gln192Arg) | rs662, | | F |
| | | | | (2003/8/7 site | | |
| | | | | correction 575 | | |
| | | | | →584) | | |
| 41 | PAR2 | PPAR gamma | C/G(Pro12Ala) | rs1801282 | | F |
| 42 | HL1 | hepatic lipase | C-480T | rs1800588 | 17 | F |
| 43 | APE2 | Apolipoprotein E | T/C(Cys112Arg) | rs429358 | 30 | F |
| 44 | MTP1 | microsomal trigyceride | G-493T | rs1800591 | 31 | F |
| | | transfer protein | | | | |
| 45 | LPL3 | Lipoprotein lipase | C/G(Ser 447 STOP) | rs328 | | F |
| 46 | ESRa1 | alfa estrogen receptor | T/C(P vuII) | rs2234693 | 18 | G |
| 47 | S2AR | serotonin 2A receptor | T102C | rs6313 | 19 | G |
| 48 | GCLM1 | glutamate-cysteine | C588T | 2670th of | 32 | G |
| | | ligase, modifier subunit | | U72210 | | |
| 49 | MMP71 | matrilysin promoter | A-181G | 1022th of | 20 | G |
| | | | | L22525 | | |

The names of references corresponding to Reference Numbers in Tables 2-1, 2-2, 7-1 and 7-2 are as follows.
1. Yoshimuura M, Nakayama M, Shimasaki Y, Ogawa H, Kugiyama K, Nakamura S, Ito T, Mizuno Y, Harada E, Yasue H, Miyamoto Y, Saito Y, Nakao K. A T-786->C mutation in the 5′-flanking region of the endothelial nitric oxide synthase gene and coronary arterial vasomotility. Am J Cardiol. 2000 85(6):7104.
2. Shimomura H, Sanke T, Ueda K, Hanabusa T, Sakagashira S, Nanjo K. A missense mutation of the muscle glycogen synthase gene (M416V) is associated with insulin resistance in the Japanese population. Diabetologia. 40(8):947-52.
3. Renner W, Schallmoser K, Gallippi P, Krauss C, Toplak H, Wascher T C, Pilger E. C242T polymorphism of the p22 phox gene is not -associated with peripheral arterial occlusive disease. Atherosclerosis. 152(1):175-9.
4. Rosmond R, Chagnon M, Bouchard C, Bjomtorp P. Increased Abdominal Obesity, Insulin and Glucose Levels in Nondiabetic Subjects with a T29C Polymorphism of the Transforming Growth Factor-beta(1) Gene. Horm Res. 59(4):191-4.
5. Blankenberg S, Rupprecht H J, Poirier O, Bickel C, Smieja M, Hafner G, Meyer J, Cambien F, Tiret L. Plasma concentrations and genetic variation of matrix metalloproteinase 9 and prognosis of patients with cardiovascular disease. Circulation. 107(12):1579-85.
6. Hofmann M A, Drury S, Hudson B I, Gleason M R, Qu W, Lu Y, Lalla E, Chitnis S, Monteiro J, Stickland M H, Bucciarelli L G, Moser B, Moxley G, Itescu S, Grant P J, Gregersen P K, Stern D M, Schmidt A M. RAGE and arthritis: the G82S polymorphism amplifies the inflammatory response. Genes Immun. 3(3):123-35.
7. Omori K, Kazama J J, Song J, Goto S, Takada T, Saito N, Sakatsume M, Narita I, Gejyo F. Association of the MCP-1 gene polymorphism A-2518G with carpal-tunnel syndrome in hemodialysis patients. Amyloid. 9(3):175-82.
8. Cascorbi I, Henning S, Brockmoller J, Gephart J, Meisel C, Muller J M, Loddenkemper R, Roots I. Substantially reduced risk of cancer of the aerodigestive tract in subjects with variant—463A of the myeloperoxidase gene. Cancer Res. (3):644-9.
9. Rauchhaus M, Gross M, Schulz S, Francis D P, Greiser P, Norwig A, Weidhase L, Coats A J, Dietz R, Anker S D, Glaser C. The E-selectin SER128ARG gene polymorphism and restenosis after successful coronary angioplasty. Int J Cardiol. 83(3):249-57.
10. Reinhardt D, Sigusch H H, Vogt S F, Zeiss C, Farker K, Hoffmann A, Muller S. A common variant of the angiotensinogen gene and the risk of coronary artery disease in a German population. Pharmazie. 55(1):69-71.
11. Kroll H, Gardemann A, Fechter A, Haberbosch W, Santoso S. The impact of the glycoprotein Ia collagen receptor subunit A1648G gene polymorphism on coronary artery disease and acute myocardial infarction. Thromb Haemost. 83(3):392-6.
12. von Beckerath N, Koch W, Mehilli J, Bottiger C, Schomig A, Kastrati A. Glycoprotein Ia gene C807T polymorphism and risk for major adverse cardiac events within the first 30 days after coronary artery stenting. Blood. 95(11):3297-301.
13. Park H Y, Nabika T, Jang Y, Kwon H M, Cho S Y, Masuda J. Association of G-33A polymorphism in the thrombomodulin gene with myocardial infarction in Koreans. Hypertens Res. 25(3):389-94.
14. Blake G J, Schmitz C, Lindpaintner K, Ridker P M. Mutation in the promoter region of the beta-fibrinogen gene and the risk of future myocardial infarction, stroke and venous thrombosis. Eur Heart J. 22(24):2262-6.
15. Jeng J R. Association of PAI-1 gene promoter 4 g/5 g polymorphism with plasma PAI-1 activity in Chinese patients with and without hypertension. Am J Hypertens. 16(4):290-6.
16. Cenarro A, Artieda M, Castillo S, Mozas P, Reyes G, Tejedor D, Alonso R, Mata P, Pocovi M, Civeira F. A common variant in the ABCA1 gene is associated with a lower risk for premature coronary heart disease in familial hypercholesterolaemia. J Med Genet. 40(3):163-8.
17. Jansen H, Chu G, Ehnholm C, Dallongeville J, Nicaud V, Talmud P J. The T allele of the hepatic lipase promoter variant C480T is associated with increased fasting lipids and HDL and increased preprandial and postprandial LpCIII:B: European Atherosclerosis Research Study (EARS) II. Arterioscler Thromb Vasc Biol. 19(2):303-8.
18. Yamada Y, Ando F, Niino N, Ohta S, Shimokata H. Association of polymorphisms of the estrogen receptor alpha gene with bone mineral density of the femoral neck in elderly Japanese women. J Mol Med. 80(7):452-60.
19. Bjork J M, Moeller F G, Dougherty D M, Swann A C, Machado M A, Hanis C L. Serotonin 2a receptor T102C polymorphism and impaired impulse control. Am J Med Genet. 114(3):336-9.
20. Jormsjo S, Whatling C, Walter D H, Zeiher A M, Hamsten A, Eriksson P. Allele-specific regulation of matrix metalloproteinase-7 promoter activity is associated with coronary artery luminal dimensions among hypercholesterolemic patients. Arterioscler Thromb Vasc Biol. 21(11):1834-9.
21. Yeh H I, Chou Y, Liu H F, Chang S C, Tsai C H. Connexin37 gene polymorphism and coronary artery disease in Taiwan. Int J Cardiol. 81(2-3):251-5.
22. Zheng F, Chevalier J A, Zhang L Q, Virgil D, Ye S Q, Kwiterovich P O. An HphI polymorphism in the E-selectin gene is associated with premature coronary artery disease. Clin Genet. 59(1):58-64
23. Wang A Y, Chan J C, Wang M, Poon E, Lui S F, Li P K, Sanderson J. Cardiac hypertrophy and remodeling in relation to ACE and angiotensinogen genes genotypes in Chinese dialysis patients. Kidney Int. 63(5):1899-907.
24. Pihusch R, Buchholz T, Lohse P, Rubsamen H, Rogenhofer N, Hasbargen U, Hiller E, Thaler C J. Thrombophilic gene mutations and recurrent spontaneous abortion: prothrombin mutation increases the risk in the first trimester. Am J Reprod Immunol. 46(2):124-31.
25. Krikovszky D, Vasarhelyi B, Toth-Heyn P, Korner A, Tulassay T, Madacsy L. Association between G-308A polymorphism of the tumor necrosis factor-alpha gene and 24-hour ambulatory blood pressure values in type 1 diabetic adolescents. Clin Genet. 62(6):474-7.
26. Cao H, Hegele R A. Human C-reactive protein (CRP) 1059G/C polymorphism. J Hum Genet. 45(2):100-1.
27. Okumura K, Matsui H, Ogawa Y, Takahashi R, Matsubara K, Imai H, Imamura A, Mizuno T, Tsuzuki M, Kitamura Y. The polymorphism of the beta3-adrenergic receptor gene is associated with reduced low-density lipoprotein particle size. Metabolism. 52(3):356-61.
28. Simsek S, Bleeker P M, van der Schoot C E, von dem Borne A E. Association of a variable number of tandem repeats (VNTR) in glycoprotein Ib alpha and HPA-2 alloantigens. Thromb Haemost. 72(5):757-61.
29. Carter A M, Catto A J, Kohler H P, Ariens R A, Stickland M H, Grant P J. alpha-fibrinogen Thr312Ala polymorphism and venous thromboembolism. Blood. 96(3):1177-9.
30. Huber G, Marz W, Martin J R, Malherbe P, Richards J G, Sueoka N, Ohm T, Hoffmann M M. Characterization of transgenic mice expressing apolipoprotein E4(C112R) and apolipoprotein E4(L28P; C112R). Neuroscience. 101(1):211-8.
31. Couture P, Otvos J D, Cupples L A, Wilson P W, Schaefer E J, Ordovas J M. Absence of association between genetic variation in the promoter of the microsomal triglyceride transfer protein gene and plasma lipoproteins in the Framingham Offspring Study. Atherosclerosis. 148(2):337-43.
32. Nakamura S, Kugiyama K, Sugiyama S, Miyamoto S, Koide S, Fukushima H, Honda O, Yoshimura M, Ogawa H. Polymorphism in the 5′-flanking region of human glutamate-cysteine ligase modifier subunit gene is associated with myocardial infarction. Circulation. 25;105(25):2968-73.

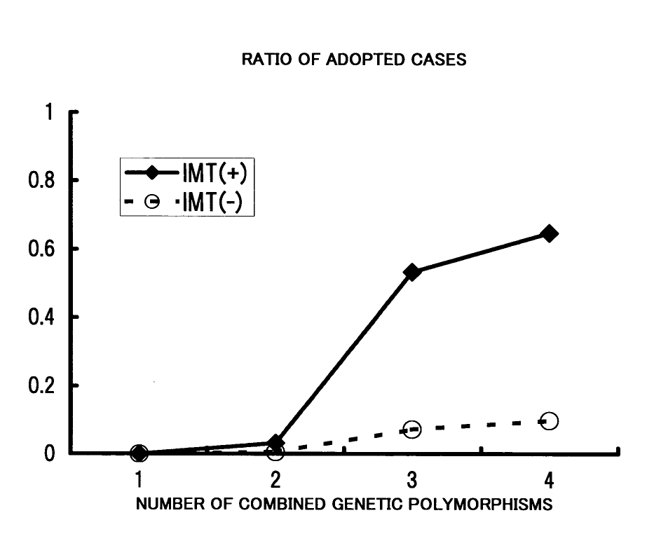

The gene arrangements corresponding to SEQ ID NO shown in Tables 2-1, 2-2, 7-1 and 7-2 are shown in FIGS. 1 and 2.

A case control study was carried out in a population of patients with diabetes having no myocardial infarction clinical history on about 437 cases with early arteriosclerosis, taking about 195 cases without early arteriosclerosis as a control. In a population of cases with myocardial infarction clinical history, myocardial infarction-related genes having a relation with myocardial infarction may also be detected, to thereby deteriorate the sensitivity of a set of genetic polymorphisms specific to arteriosclerosis. To avoid this, the study was carried out on the population of cases having no myocardial infarction clinical history. Thus, the detected sets of arteriosclerosis-associated genetic polymorphisms were specific to arteriosclerosis and contribute to prediction of the risk of arteriosclerosis.

IMTs were measured by the measurement method of Example 1. Subjects having measured IMT 0.2 mm or more larger (SD=0.1) than the average IMT of the healthy subjects were classified into a group of subjects with early arteriosclerosis. The significance level was set at an odds ratio of 10 or more and a chi-square of 6.635 (P<0.01) or more, and sets of arteriosclerosis-associated genetic polymorphisms were decided (Tables 8-1 to 8-4).

TABLE 8-1

| | Polymorphism | | Polymorphism | | Polymorphism | | | Odds | Chi- |
|---|---|---|---|---|---|---|---|---|---|
| Disease | No. | Category | No. | Category | No. | Category | Incidence | Ratio | Square |
| IMT | 3 | 12 | 46 | 12 | | | 0.034 | 99 | 6.63 |
| IMT | 1 | 3 | 21 | 1 | 28 | 12 | 0.067 | 13.1 | 10.51 |
| IMT | 1 | 3 | 28 | 12 | 29 | 1 | 0.064 | 12.4 | 9.82 |
| IMT | 1 | 3 | 28 | 12 | 40 | 12 | 0.053 | 10.3 | 7.82 |
| IMT | 1 | 3 | 28 | 12 | 34 | 23 | 0.057 | 10.7 | 8.25 |
| IMT | 3 | 12 | 4 | 1 | 7 | 23 | 0.036 | 99 | 6.88 |
| IMT | 3 | 12 | 7 | 23 | 17 | 12 | 0.041 | 99 | 7.56 |
| IMT | 3 | 12 | 7 | 23 | 31 | 12 | 0.038 | 99 | 7.35 |
| IMT | 3 | 12 | 7 | 23 | 34 | 12 | 0.045 | 99 | 8.38 |
| IMT | 3 | 12 | 8 | 3 | 46 | 12 | 0.034 | 99 | 6.64 |
| IMT | 3 | 12 | 9 | 12 | 46 | 12 | 0.035 | 99 | 6.64 |
| IMT | 3 | 12 | 12 | 23 | 31 | 12 | 0.055 | 10.3 | 7.86 |
| IMT | 3 | 12 | 12 | 23 | 46 | 12 | 0.039 | 99 | 7.13 |
| IMT | 3 | 12 | 13 | 23 | 46 | 12 | 0.034 | 99 | 6.64 |
| IMT | 3 | 12 | 14 | 23 | 46 | 12 | 0.035 | 99 | 6.73 |
| IMT | 3 | 12 | 15 | 12 | 25 | 12 | 0.052 | 10.1 | 7.68 |
| IMT | 3 | 12 | 15 | 12 | 46 | 12 | 0.034 | 99 | 6.64 |
| IMT | 3 | 12 | 22 | 12 | 31 | 12 | 0.055 | 10.5 | 8.05 |
| IMT | 3 | 12 | 24 | 12 | 46 | 12 | 0.035 | 99 | 6.71 |
| IMT | 3 | 12 | 25 | 12 | 31 | 12 | 0.047 | 99 | 9.06 |
| IMT | 3 | 12 | 26 | 3 | 46 | 12 | 0.035 | 99 | 6.72 |
| IMT | 3 | 12 | 28 | 23 | 31 | 12 | 0.051 | 10 | 7.61 |
| IMT | 3 | 12 | 28 | 23 | 46 | 12 | 0.035 | 99 | 6.67 |
| IMT | 3 | 12 | 29 | 12 | 46 | 12 | 0.035 | 99 | 6.72 |
| IMT | 3 | 12 | 30 | 12 | 46 | 12 | 0.034 | 99 | 6.66 |
| IMT | 3 | 12 | 31 | 12 | 41 | 1 | 0.052 | 10.4 | 7.93 |
| IMT | 3 | 12 | 41 | 1 | 42 | 12 | 0.052 | 10.4 | 7.94 |
| IMT | 3 | 12 | 41 | 1 | 46 | 12 | 0.034 | 99 | 6.65 |
| IMT | 3 | 12 | 43 | 23 | 46 | 12 | 0.035 | 99 | 6.67 |
| IMT | 3 | 12 | 44 | 12 | 46 | 12 | 0.035 | 99 | 6.67 |
| IMT | 3 | 12 | 42 | 12 | 47 | 23 | 0.038 | 99 | 7.33 |
| IMT | 3 | 12 | 46 | 12 | 49 | 12 | 0.035 | 99 | 6.66 |
| IMT | 4 | 1 | 7 | 12 | 28 | 12 | 0.054 | 10.5 | 8.04 |
| IMT | 4 | 1 | 28 | 12 | 40 | 12 | 0.055 | 10.7 | 8.27 |
| IMT | 4 | 23 | 25 | 1 | 40 | 12 | 0.043 | 99 | 8.2 |
| IMT | 6 | 1 | 7 | 1 | 20 | 1 | 0.036 | 99 | 6.65 |
| IMT | 7 | 1 | 37 | 3 | 45 | 1 | 0.037 | 99 | 7.05 |
| IMT | 11 | 3 | 20 | 3 | 32 | 12 | 0.05 | 99 | 9.29 |
| IMT | 11 | 1 | 35 | 1 | 38 | 3 | 0.05 | 99 | 9.19 |
| IMT | 11 | 1 | 38 | 3 | 40 | 12 | 0.05 | 99 | 9.29 |
| IMT | 17 | 1 | 36 | 12 | 37 | 3 | 0.039 | 99 | 7.18 |
| IMT | 17 | 1 | 37 | 3 | 48 | 1 | 0.04 | 99 | 7.3 |
| IMT | 17 | 23 | 28 | 12 | 40 | 12 | 0.039 | 99 | 7.26 |
| IMT | 20 | 3 | 22 | 1 | 24 | 23 | 0.059 | 11.3 | 8.74 |

TABLE 8-2

| | Poly- | | Poly- | | Poly- | | Poly- | | | | |
|---|---|---|---|---|---|---|---|---|---|---|---|
| | morphism | | morphism | | morphism | | morphism | | | Odds | Chi- |
| Disease | No. | Category | No. | Category | No. | Category | No. | Category | Incidence | Ratio | square |
| IMT | 20 | 3 | 24 | 23 | 33 | 12 | | | 0.054 | 10.7 | 8.24 |
| IMT | 20 | 3 | 24 | 23 | 38 | 12 | | | 0.053 | 10.2 | 7.8 |
| IMT | 20 | 3 | 24 | 23 | 48 | 1 | | | 0.059 | 11.5 | 8.97 |
| IMT | 17 | 1 | 20 | 3 | 32 | 12 | | | 0.049 | 99 | 9 |
| IMT | 20 | 3 | 32 | 12 | 40 | 12 | | | 0.067 | 13.3 | 10.67 |
| IMT | 20 | 3 | 32 | 12 | 38 | 3 | | | 0.037 | 99 | 6.9 |
| IMT | 21 | 1 | 28 | 12 | 40 | 12 | | | 0.052 | 10.2 | 7.78 |
| IMT | 11 | 12 | 25 | 1 | 38 | 3 | | | 0.059 | 11.1 | 8.55 |
| IMT | 17 | 1 | 25 | 1 | 38 | 3 | | | 0.04 | 99 | 7.24 |
| IMT | 23 | 12 | 25 | 1 | 38 | 3 | | | 0.039 | 99 | 7.13 |
| IMT | 25 | 1 | 36 | 1 | 38 | 3 | | | 0.046 | 99 | 8.55 |
| IMT | 25 | 1 | 38 | 3 | 40 | 12 | | | 0.065 | 12.4 | 9.85 |
| IMT | 28 | 12 | 32 | 3 | 40 | 12 | | | 0.053 | 10.1 | 7.7 |
| IMT | 7 | 12 | 28 | 12 | 33 | 12 | | | 0.044 | 99 | 8.4 |
| IMT | 28 | 12 | 38 | 23 | 40 | 12 | | | 0.053 | 10.2 | 7.77 |
| IMT | 31 | 3 | 32 | 3 | 47 | 1 | | | 0.043 | 99 | 8.19 |
| IMT | 4 | 12 | 31 | 3 | 45 | 23 | | | 0.054 | 10.5 | 8.02 |
| IMT | 15 | 1 | 31 | 3 | 45 | 23 | | | 0.046 | 99 | 8.91 |
| IMT | 31 | 3 | 45 | 23 | 46 | 12 | | | 0.035 | 99 | 6.64 |
| IMT | 34 | 3 | 36 | 12 | 39 | 12 | | | 0.038 | 99 | 7.06 |
| IMT | 34 | 3 | 37 | 1 | 47 | 1 | | | 0.036 | 99 | 6.66 |
| IMT | 17 | 12 | 34 | 3 | 39 | 12 | | | 0.037 | 99 | 6.77 |
| IMT | 34 | 3 | 39 | 12 | 41 | 1 | | | 0.04 | 99 | 7.55 |
| IMT | 34 | 3 | 39 | 12 | 45 | 1 | | | 0.04 | 99 | 7.58 |
| IMT | 16 | 1 | 34 | 1 | 40 | 1 | | | 0.035 | 99 | 6.7 |
| IMT | 18 | 1 | 34 | 1 | 40 | 1 | | | 0.036 | 99 | 6.64 |
| IMT | 24 | 1 | 34 | 1 | 40 | 1 | | | 0.039 | 99 | 7.29 |
| IMT | 34 | 1 | 40 | 1 | 45 | 1 | | | 0.036 | 99 | 6.73 |
| IMT | 12 | 12 | 39 | 12 | 43 | 3 | | | 0.036 | 99 | 6.68 |
| IMT | 1 | 23 | 11 | 3 | 31 | 23 | 39 | 12 | 0.039 | 99 | 7.28 |
| IMT | 2 | 1 | 7 | 3 | 20 | 3 | 38 | 3 | 0.049 | 99 | 8.76 |
| IMT | 2 | 1 | 25 | 12 | 38 | 3 | 39 | 12 | 0.043 | 99 | 7.54 |
| IMT | 2 | 1 | 33 | 23 | 38 | 3 | 39 | 12 | 0.041 | 99 | 7.4 |
| IMT | 2 | 1 | 11 | 3 | 31 | 23 | 39 | 12 | 0.038 | 99 | 6.64 |
| IMT | 2 | 1 | 35 | 1 | 38 | 3 | 39 | 12 | 0.042 | 99 | 7.5 |
| IMT | 4 | 23 | 20 | 3 | 33 | 12 | 40 | 12 | 0.045 | 99 | 8.68 |
| IMT | 4 | 23 | 23 | 3 | 33 | 12 | 40 | 12 | 0.055 | 10.8 | 8.32 |
| IMT | 4 | 23 | 33 | 12 | 40 | 12 | 47 | 12 | 0.059 | 99 | 11.49 |
| IMT | 4 | 23 | 11 | 3 | 20 | 3 | 40 | 12 | 0.037 | 99 | 6.93 |
| IMT | 4 | 23 | 20 | 3 | 31 | 23 | 40 | 12 | 0.035 | 99 | 6.77 |
| IMT | 4 | 23 | 29 | 1 | 38 | 3 | 40 | 12 | 0.036 | 99 | 6.67 |
| IMT | 5 | 1 | 11 | 3 | 39 | 12 | 43 | 3 | 0.053 | 10.1 | 7.65 |
| IMT | 5 | 1 | 12 | 12 | 20 | 3 | 23 | 3 | 0.048 | 99 | 8.83 |
| IMT | 5 | 1 | 15 | 1 | 20 | 3 | 32 | 12 | 0.054 | 10.4 | 7.99 |

TABLE 8-3

| | Poly- | | Poly- | | Poly- | | Poly- | | | | |
|---|---|---|---|---|---|---|---|---|---|---|---|
| | morphism | | morphism | | morphism | | morphism | | | Odds | Chi- |
| Disease | No. | Category | No. | Category | No. | Category | No. | Category | Incidence | Ratio | square |
| IMT | 5 | 1 | 20 | 3 | 23 | 3 | 31 | 3 | 0.038 | 99 | 7.25 |
| IMT | 5 | 1 | 20 | 3 | 37 | 23 | 39 | 12 | 0.063 | 12.5 | 9.92 |
| IMT | 5 | 1 | 12 | 12 | 25 | 1 | 44 | 1 | 0.037 | 99 | 6.68 |
| IMT | 5 | 1 | 25 | 1 | 39 | 12 | 45 | 1 | 0.035 | 99 | 6.65 |
| IMT | 6 | 12 | 11 | 3 | 37 | 23 | 39 | 12 | 0.038 | 99 | 6.99 |
| IMT | 6 | 12 | 11 | 3 | 39 | 12 | 43 | 3 | 0.043 | 99 | 7.97 |
| IMT | 6 | 1 | 15 | 1 | 17 | 3 | 25 | 1 | 0.037 | 99 | 6.73 |
| IMT | 6 | 1 | 17 | 3 | 33 | 12 | 40 | 12 | 0.06 | 11.5 | 8.97 |
| IMT | 6 | 1 | 11 | 3 | 20 | 1 | 42 | 12 | 0.043 | 99 | 7.95 |
| IMT | 6 | 1 | 20 | 1 | 23 | 3 | 40 | 12 | 0.036 | 99 | 6.77 |
| IMT | 6 | 23 | 9 | 1 | 22 | 23 | 25 | 1 | 0.039 | 99 | 7.08 |
| IMT | 6 | 23 | 22 | 23 | 25 | 1 | 42 | 12 | 0.042 | 99 | 7.65 |
| IMT | 6 | 23 | 20 | 3 | 25 | 1 | 46 | 12 | 0.052 | 99 | 9.83 |
| IMT | 7 | 3 | 9 | 12 | 20 | 3 | 38 | 3 | 0.054 | 99 | 10.03 |
| IMT | 7 | 3 | 13 | 3 | 20 | 3 | 38 | 3 | 0.05 | 99 | 9.47 |
| IMT | 7 | 3 | 14 | 3 | 20 | 3 | 38 | 3 | 0.053 | 99 | 10.09 |
| IMT | 7 | 3 | 20 | 3 | 22 | 12 | 38 | 3 | 0.053 | 99 | 9.45 |
| IMT | 7 | 3 | 20 | 3 | 23 | 3 | 38 | 3 | 0.046 | 99 | 8.55 |
| IMT | 7 | 3 | 20 | 3 | 27 | 3 | 38 | 3 | 0.055 | 99 | 10.35 |
| IMT | 7 | 3 | 20 | 3 | 29 | 1 | 38 | 3 | 0.042 | 99 | 7.57 |
| IMT | 7 | 3 | 20 | 3 | 30 | 1 | 38 | 3 | 0.053 | 99 | 10.07 |
| IMT | 7 | 3 | 20 | 3 | 33 | 12 | 40 | 12 | 0.062 | 99 | 12.11 |
| IMT | 7 | 3 | 20 | 3 | 35 | 1 | 38 | 3 | 0.046 | 99 | 8.58 |
| IMT | 7 | 3 | 20 | 3 | 38 | 3 | 49 | 12 | 0.053 | 99 | 10.04 |
| IMT | 7 | 23 | 17 | 23 | 33 | 1 | 40 | 12 | 0.036 | 99 | 6.68 |
| IMT | 9 | 1 | 12 | 12 | 34 | 3 | 36 | 12 | 0.042 | 99 | 7.44 |
| IMT | 9 | 1 | 20 | 3 | 31 | 3 | 36 | 12 | 0.048 | 99 | 9.13 |
| IMT | 9 | 1 | 31 | 3 | 33 | 12 | 40 | 12 | 0.045 | 99 | 8.61 |
| IMT | 9 | 1 | 12 | 12 | 24 | 1 | 34 | 3 | 0.039 | 99 | 6.93 |
| IMT | 9 | 12 | 25 | 1 | 39 | 12 | 45 | 1 | 0.04 | 99 | 7.56 |
| IMT | 9 | 12 | 25 | 1 | 39 | 12 | 46 | 12 | 0.038 | 99 | 7.21 |
| IMT | 11 | 3 | 16 | 1 | 31 | 23 | 39 | 12 | 0.038 | 99 | 7.29 |
| IMT | 11 | 3 | 17 | 12 | 31 | 23 | 39 | 12 | 0.042 | 99 | 7.78 |
| IMT | 11 | 3 | 18 | 1 | 31 | 23 | 39 | 12 | 0.036 | 99 | 6.73 |
| IMT | 11 | 3 | 22 | 12 | 31 | 23 | 39 | 12 | 0.045 | 99 | 8.1 |
| IMT | 11 | 3 | 23 | 23 | 31 | 23 | 39 | 12 | 0.039 | 99 | 7.26 |
| IMT | 11 | 3 | 31 | 23 | 36 | 12 | 39 | 12 | 0.041 | 99 | 7.66 |
| IMT | 11 | 3 | 39 | 12 | 42 | 12 | 43 | 3 | 0.045 | 99 | 8.42 |
| IMT | 11 | 3 | 39 | 12 | 43 | 3 | 47 | 12 | 0.042 | 99 | 7.87 |
| IMT | 12 | 12 | 20 | 3 | 23 | 3 | 36 | 12 | 0.048 | 99 | 8.76 |
| IMT | 12 | 12 | 23 | 3 | 32 | 12 | 41 | 1 | 0.039 | 99 | 7.05 |
| IMT | 12 | 12 | 40 | 12 | 42 | 1 | 47 | 12 | 0.041 | 99 | 7.63 |
| IMT | 12 | 12 | 25 | 1 | 34 | 12 | 44 | 1 | 0.046 | 99 | 8.16 |
| IMT | 13 | 3 | 25 | 12 | 38 | 3 | 39 | 12 | 0.041 | 99 | 7.64 |

TABLE 8-4

| | Poly- | | Poly- | | Poly- | | Poly- | | | | |
|---|---|---|---|---|---|---|---|---|---|---|---|
| | morphism | | morphism | | morphism | | morphism | | | Odds | Chi- |
| Disease | No. | Category | No. | Category | No. | Category | No. | Category | Incidence | Ratio | square |
| IMT | 13 | 3 | 36 | 12 | 38 | 3 | 39 | 12 | 0.042 | 99 | 7.99 |
| IMT | 13 | 3 | 38 | 3 | 39 | 12 | 43 | 3 | 0.044 | 99 | 8.15 |
| IMT | 14 | 3 | 15 | 1 | 20 | 3 | 32 | 12 | 0.062 | 12.1 | 9.59 |
| IMT | 14 | 3 | 20 | 3 | 23 | 3 | 31 | 3 | 0.05 | 99 | 9.75 |
| IMT | 15 | 1 | 25 | 1 | 31 | 3 | 32 | 3 | 0.038 | 99 | 7.1 |
| IMT | 15 | 1 | 17 | 23 | 31 | 3 | 40 | 12 | 0.054 | 10.3 | 7.87 |
| IMT | 15 | 1 | 31 | 3 | 33 | 12 | 40 | 12 | 0.034 | 99 | 6.64 |
| IMT | 15 | 1 | 20 | 3 | 23 | 3 | 32 | 12 | 0.055 | 10.6 | 8.1 |
| IMT | 15 | 1 | 20 | 3 | 32 | 12 | 36 | 12 | 0.057 | 10.9 | 8.43 |
| IMT | 17 | 1 | 20 | 3 | 25 | 1 | 31 | 23 | 0.039 | 99 | 7.28 |
| IMT | 20 | 3 | 23 | 3 | 31 | 3 | 32 | 3 | 0.04 | 99 | 7.58 |
| IMT | 20 | 3 | 23 | 3 | 31 | 3 | 38 | 23 | 0.039 | 99 | 7.19 |
| IMT | 20 | 23 | 28 | 12 | 40 | 12 | 42 | 23 | 0.041 | 99 | 7.79 |
| IMT | 20 | 3 | 28 | 12 | 29 | 1 | 46 | 23 | 0.036 | 99 | 6.8 |
| IMT | 20 | 3 | 29 | 1 | 37 | 23 | 39 | 12 | 0.062 | 12.1 | 9.51 |
| IMT | 20 | 3 | 29 | 1 | 39 | 12 | 42 | 12 | 0.063 | 12.4 | 9.86 |
| IMT | 20 | 3 | 29 | 1 | 39 | 12 | 43 | 3 | 0.063 | 12.2 | 9.6 |
| IMT | 20 | 3 | 22 | 1 | 23 | 3 | 32 | 12 | 0.057 | 10.6 | 8.14 |
| IMT | 20 | 3 | 36 | 12 | 37 | 23 | 39 | 12 | 0.056 | 10.8 | 8.29 |
| IMT | 20 | 3 | 36 | 12 | 39 | 12 | 45 | 1 | 0.06 | 11.6 | 9.07 |
| IMT | 20 | 3 | 38 | 3 | 39 | 12 | 43 | 3 | 0.036 | 99 | 6.68 |
| IMT | 20 | 3 | 23 | 3 | 32 | 12 | 46 | 12 | 0.059 | 11.3 | 8.77 |
| IMT | 20 | 3 | 37 | 23 | 39 | 12 | 46 | 12 | 0.053 | 99 | 10.37 |
| IMT | 20 | 3 | 39 | 12 | 42 | 12 | 46 | 12 | 0.057 | 11.3 | 8.81 |
| IMT | 20 | 3 | 39 | 12 | 45 | 1 | 46 | 12 | 0.055 | 10.8 | 8.34 |
| IMT | 20 | 3 | 39 | 12 | 46 | 12 | 47 | 12 | 0.057 | 11.2 | 8.73 |
| IMT | 20 | 3 | 37 | 23 | 39 | 12 | 48 | 1 | 0.058 | 11.1 | 8.61 |
| IMT | 23 | 3 | 37 | 3 | 38 | 3 | 48 | 1 | 0.038 | 99 | 6.74 |
| IMT | 23 | 3 | 31 | 23 | 39 | 12 | 47 | 12 | 0.057 | 11.1 | 8.62 |
| IMT | 23 | 3 | 37 | 23 | 39 | 12 | 47 | 12 | 0.063 | 12.3 | 9.74 |
| IMT | 25 | 1 | 34 | 12 | 38 | 3 | 44 | 1 | 0.057 | 10.5 | 8.02 |
| IMT | 25 | 1 | 33 | 23 | 39 | 12 | 45 | 1 | 0.037 | 99 | 7.06 |
| IMT | 25 | 1 | 39 | 12 | 45 | 1 | 47 | 12 | 0.038 | 99 | 7.08 |
| IMT | 25 | 1 | 33 | 23 | 39 | 12 | 46 | 12 | 0.035 | 99 | 6.76 |
| IMT | 28 | 12 | 29 | 1 | 33 | 12 | 40 | 12 | 0.038 | 99 | 7.25 |
| IMT | 28 | 12 | 29 | 1 | 40 | 12 | 42 | 23 | 0.041 | 99 | 7.77 |
| IMT | 28 | 12 | 34 | 23 | 40 | 12 | 42 | 23 | 0.041 | 99 | 7.68 |
| IMT | 28 | 12 | 33 | 12 | 40 | 12 | 48 | 1 | 0.039 | 99 | 7.27 |
| IMT | 28 | 12 | 40 | 12 | 42 | 23 | 48 | 1 | 0.044 | 99 | 8.31 |
| IMT | 31 | 3 | 32 | 3 | 33 | 12 | 37 | 23 | 0.055 | 10.5 | 8 |
| IMT | 35 | 1 | 38 | 3 | 39 | 12 | 43 | 3 | 0.042 | 99 | 7.74 |
| IMT | 35 | 1 | 37 | 3 | 44 | 23 | 48 | 1 | 0.037 | 99 | 6.73 |
| IMT | 35 | 1 | 39 | 12 | 43 | 3 | 44 | 23 | 0.041 | 99 | 7.83 |
| IMT | 36 | 1 | 37 | 3 | 40 | 12 | 42 | 23 | 0.041 | 99 | 7.66 |

The odds ratio (Odd) herein is an indicator showing how often a corresponding event occurs as compared with the control group. An “odds ratio of 2” indicates that, for example, one has a tendency to develop arteriosclerosis twice that in the control group. “odds ratio of 99” indicates that the event does not occur in the control group. Chi-square (Chi) is an indicator showing the significant difference where the event occurs and a chi square of 6.635 or more corresponds to P<0.01.

No genetic polymorphism that can explain early arteriosclerosis (Odd>10 and Chi>6.635) was found among the searched 49 genetic polymorphisms as a result of analysis on a single genetic polymorphism (49×4=196 genotypes). The genotypes of genetic polymorphisms were classified in the following manner. When the genotype is, for example, represented by MPO (G463A), the homozygosis (AA) of the polymorphism having an anterior base in alphabetic order of substituted bases was indicated as genotype 1, its heterozygosis (GA) was indicated as genotype 2, the homozygous (GG) of the polymorphism having a posterior base in alphabetic order of substituted bases was indicated as genotype 3. (A is anterior to G in alphabetic order. ) The analyses were carried out on the four groups of genotype 1, genotypes 1-2, genotypes 2-3, and, genotype 3. In this procedure, a group which shows at least a positive significant difference when combined with other genotypes of genetic polymorphisms was adopted. For example, if both the genotype 1 and genotypes 1-2 showed a significance, one having a higher significance was adopted.

Combinations of two genetic polymorphisms were then searched (49×48/2×4×4=17186 sets of genetic polymorphisms). One set of arteriosclerosis-associated genetic polymorphisms which shows Odd>10 and Chi>6.635 and can explain the relation with early arteriosclerosis was extracted. Of 437 cases with early arteriosclerosis, 14 cases could be explained by this set of genetic polymorphisms. In contrast, none of 195 non-arteriosclerosis cases had this set of genetic polymorphisms. The combination of the groups was ag.

Then, combinations of three genetic polymorphisms were searched (49×48×47/6×4×4×4<500,000 sets of genetic polymorphisms). As a result, 72 sets of arteriosclerosis-associated genetic polymorphisms which show Odd>10 and Chi >6.635 and can explain the relation with early arteriosclerosis were extracted. Of 437 cases with early arteriosclerosis, 233 cases could be explained by these sets of genetic polymorphisms. In contrast, only 14 cases of 195 non-early arteriosclerosis cases had any of these sets of genetic polymorphisms. Sets of genetic polymorphisms which had been already extracted in the search on the combinations of two genetic polymorphisms were not extracted in the search on the combinations of three genetic polymorphisms. Regarding combinations of individual groups, 21 combinations of 34 combinations included only one genetic polymorphism belonging to one of the Groups (A) to (G).

Next, combinations of four genetic polymorphisms were searched (49×48×47×46/24×4×4×4×4<15,000,000 sets of genetic polymorphisms). As a result, 103 sets of arteriosclerosis-associated genetic polymorphisms that can explain early arteriosclerosis (Odd>10 and Chi>6.635) were extracted. Of 437 cases with early arteriosclerosis, 283 cases could be explained by these sets. In contrast, only 19 cases of 195 non-early arteriosclerosis cases had any of these sets of genetic polymorphisms. Sets including the sets of genetic polymorphisms which had been already extracted in the search on the combinations of two and three genetic polymorphisms were not extracted in the search on the combinations of four genetic polymorphisms. Regarding combinations of individual groups, 200 combinations of 706 combinations included only one genetic polymorphism belonging to one of the Groups (A) to (G).

FIG. 1 shows the ratio of cases that could be explained by the set of arteriosclerosis-associated genetic polymorphisms among early arteriosclerosis cases, and the ratio of cases that do not develop symptom even having the set of arteriosclerosis-associated genetic polymorphisms among cases without early arteriosclerosis. FIG. 1 shows that the ratio may possibly achieve a state of equilibrium at a number of the combined genetic polymorphisms of five sets. Accordingly, the number of genetic polymorphisms enough to constitute a combination is about 3 to about 5.

### Example 5

Next, based on the above results, further investigations were made in a wilder range of genetic polymorphisms on various combinations of the genetic polymorphisms. Up to two sets of genetic polymorphisms were combined, and sets of arteriosclerosis-associated genetic polymorphisms were decided and whether or not the disease onset can be predicted was examined.

A case control study was carried out in a population of patients with diabetes having no myocardial infarction clinical history on about 405 cases with early arteriosclerosis, taking about 367 cases without early arteriosclerosis as a control. IMTs were measured by the measurement method of Example 1. Subjects having measured IMT 0.2 mm or more larger (SD=0.1) than the average IMT of the healthy subjects were classified into a group of subjects with early arteriosclerosis. According to the present example, 126 genetic polymorphisms containing 49 genetic polymorphisms extracted in the Example 4 were investigated. The significance level was set at an odds ratio of 3 or more and a chi-square of 6.635 (P<0.01) or more, and sets of arteriosclerosis-associated genetic polymorphisms, which are combinations of two genetic polymorphisms, were extracted (Tables 9-1 and 9-2).

TABLE 9-1

| Poly- | | | Poly- | | | | | |
|---|---|---|---|---|---|---|---|---|
| morphism | | Name of Genetic | morphism | | Name of Genetic | | Chi- | Odds |
| No. | Category | Polymorphism | No. | Category | Polymorphism | Frequency | square | Ratio |
| 42 | 12 | hepatic_lipase | 61 | 1 | RAGE(Gly82Ser) | 0.016 | 11.8 | 99 |
| 41 | 1 | PPAR_gamma | 61 | 1 | RAGE(Gly82Ser) | 0.014 | 9.7 | 99 |
| 18 | 23 | E-selectin | 58 | 1 | LTA(C804A(Thr26Asn)) | 0.014 | 9.6 | 99 |
| 18 | 23 | E-selectin | 57 | 3 | LTA(A252G) | 0.013 | 9.4 | 99 |
| 61 | 1 | RAGE(Gly82Ser) | 70 | 12 | Thrombopoietin(A5713G) | 0.013 | 9 | 99 |
| 7 | 12 | TGF beta | 61 | 1 | RAGE(Gly82Ser) | 0.012 | 8.8 | 99 |
| 2 | 23 | Enos 298 | 11 | 1 | MCP-1(A-2518G) | 0.011 | 7.9 | 99 |
| 27 | 3 | GPIIbIIIa | 61 | 1 | RAGE(Gly82Ser) | 0.018 | 9.1 | 11.9 |
| 61 | 1 | RAGE(Gly82Ser) | 63 | 12 | CYP2C9_3(Leu359Ile) | 0.018 | 9 | 11.8 |
| 61 | 1 | RAGE(Gly82Ser) | 68 | 23 | IL-10(C-819T) | 0.017 | 8.3 | 11.1 |
| 10 | 12 | MMP-12 | 61 | 1 | RAGE(Gly82Ser) | 0.016 | 8.1 | 10.8 |
| 47 | 12 | serotonin_2A_receptor | 61 | 1 | RAGE(Gly82Ser) | 0.016 | 8 | 10.8 |
| 6 | 12 | interleukin6(C-634G) | 61 | 1 | RAGE(Gly82Ser) | 0.015 | 7.3 | 10 |
| 51 | 23 | FactorXII | 61 | 1 | RAGE(Gly82Ser) | 0.015 | 7.1 | 9.8 |
| 38 | 3 | ABCA1 | 20 | 1 | ACE | 0.02 | 7.6 | 6.3 |
| 20 | 1 | ACE | 52 | 3 | glycoproteinIa(C807T) | 0.02 | 7.5 | 6.3 |
| 37 | 3 | MTHFR(C677T) | 58 | 1 | LTA(C804A(Thr26Asn)) | 0.029 | 10.4 | 6 |
| 20 | 1 | ACE | 54 | 1 | GP Ia(G873A) | 0.019 | 7 | 6 |
| 37 | 3 | MTHFR(C677T) | 57 | 3 | LTA(A252G) | 0.027 | 9.2 | 5.5 |
| 1 | 23 | Enos786 | 57 | 3 | LTA(A252G) | 0.134 | 12.7 | 4.4 |
| 60 | 3 | Adiponectin(G276T) | 66 | 23 | Methionine | 0.03 | 8.3 | 4.4 |
| | | | | | synthase(A2756G(Asp919gly)) | | | |
| 36 | 12 | PAI-1 | 62 | 1 | Thrombospondin-1(A2210G) | 0.482 | 88.9 | 4.3 |
| 57 | 3 | LTA(A252G) | 69 | 3 | IL-18(G-137C) | 0.111 | 10.4 | 4.3 |
| 58 | 1 | LTA(C804A(Thr26Asn)) | 69 | 3 | IL-18(G-137C) | 0.111 | 10.4 | 4.3 |
| 7 | 12 | TGF beta | 62 | 1 | Thrombospondin-1(A2210G) | 0.395 | 76.9 | 4 |
| 49 | 1 | matrilyn | 62 | 1 | Thrombospondin-1(A2210G) | 0.488 | 79 | 3.9 |
| | | promoter(A-181G) | | | | | | |
| 62 | 1 | Thrombospondin-1(A2210G) | 71 | 1 | LDL receptor related | 0.451 | 77.9 | 3.9 |
| | | | | | protein(C766T) | | | |
| 21 | 1 | AT2-receptor | 62 | 1 | Thrombospondin-1(A2210G) | 0.482 | 77 | 3.8 |
| 62 | 1 | Thrombospondin-1(A2210G) | 68 | 23 | IL-10(C-819T) | 0.492 | 76.7 | 3.8 |
| 55 | 12 | bradykinin B2 | 62 | 1 | Thrombospondin-1(A2210G) | 0.381 | 68.1 | 3.8 |
| | | receptor(C-58T) | | | | | | |
| 57 | 3 | LTA(A252G) | 64 | 1 | interleukin 1 beta(C3953T) | 0.139 | 11.5 | 3.8 |

TABLE 9-2

| Poly- | | | Poly- | | | | | |
|---|---|---|---|---|---|---|---|---|
| morphism | | Name of Genetic | morphism | | | | Chi- | Odds |
| No. | Category | Polymorphism | No. | Category | Name of Genetic Polymorphism | Frequency | square | Ratio |
| 50 | 1 | p22phox | 62 | 1 | Thrombospondin-1(A2210G) | 0.481 | 73.6 | 3.7 |
| 24 | 1 | beta2 Adrenoreceptor(C79T) | 62 | 1 | Thrombospondin-1(A2210G) | 0.482 | 72.6 | 3.7 |
| 53 | 1 | apolipoproteinE(E3 | 57 | 3 | LTA(A252G) | 0.14 | 10.7 | 3.7 |
| | | inexon 4(Arg 158Cys) | | | | | | |
| 51 | 23 | FactorXII | 62 | 1 | Thrombospondin-1(A2210G) | 0.499 | 72.8 | 3.6 |
| 62 | 1 | Thrombospondin-1(A2210G) | 65 | 12 | IL-18(C-607A) | 0.439 | 68.2 | 3.6 |
| 8 | 3 | TNFalfa(G-238A) | 57 | 3 | LTA(A252G) | 0.149 | 11.6 | 3.6 |
| 57 | 3 | LTA(A252G) | 59 | 23 | Thrombospondin4(G1186C(Ala387Pro)) | 0.147 | 11.4 | 3.6 |
| 1 | 23 | Enos786 | 58 | 1 | LTA(C804A(Thr26Asn)) | 0.135 | 10.6 | 3.6 |
| 68 | 23 | IL-10(C-819T) | 72 | 1 | PGC-1(G1302A(Thr394Thr)) | 0.044 | 9.9 | 3.6 |
| 29 | 23 | HPA-2(Thr145Met) | 40 | 1 | PON1(Gly192Arg) | 0.031 | 7 | 3.6 |
| 42 | 12 | hepatic_lipase | 62 | 1 | Thrombospondin-1(A2210G) | 0.425 | 67 | 3.5 |
| 17 | 12 | ICAM1(E469K) | 62 | 1 | Thrombospondin-1(A2210G) | 0.457 | 66.4 | 3.5 |
| 47 | 12 | serotonin_2A_receptor | 62 | 1 | Thrombospondin-1(A2210G) | 0.401 | 64 | 3.5 |
| 14 | 23 | CRP(G1059C) | 57 | 3 | LTA(A252G) | 0.145 | 11.2 | 3.5 |
| 41 | 1 | PPAR_gamma | 72 | 1 | PGC-1(G1302A(Thr394Thr)) | 0.043 | 9.2 | 3.5 |
| 6 | 12 | interleukin6(C-634G) | 62 | 1 | Thrombospondin-1(A2210G) | 0.493 | 64.2 | 3.4 |
| 25 | 12 | beta-adrenergic | 62 | 1 | Thrombospondin-1(A2210G) | 0.42 | 64 | 3.4 |
| | | receptor(A46G) | | | | | | |
| 57 | 3 | LTA(A252G) | 65 | 12 | IL-18(C-607A) | 0.076 | 16 | 3.4 |
| 26 | 23 | HANP(T2238C) | 57 | 3 | LTA(A252G) | 0.143 | 10.7 | 3.4 |
| 62 | 1 | Thrombospondin-1(A2210G) | 67 | 23 | von Willebrand Factor (G-1051A) | 0.415 | 56.9 | 3.2 |
| 58 | 1 | LTA(C804A(Thr26Asn)) | 65 | 12 | IL-18(C-607A) | 0.078 | 15.1 | 3.2 |
| 58 | 1 | LTA(C804A(Thr26Asn)) | 64 | 1 | interleukin 1 beta(C3953T) | 0.137 | 9.2 | 3.2 |
| 56 | 3 | resistin(ATG repeat) | 57 | 3 | LTA(A252G) | 0.137 | 9 | 3.2 |
| 36 | 12 | PAI-1 | 72 | 1 | PGC-1(G1302A(Thr394Thr)) | 0.041 | 7.9 | 3.2 |
| 35 | 23 | beta Fib(C148T) | 58 | 1 | LTA(C804A(Thr26Asn)) | 0.039 | 7.5 | 3.2 |
| 58 | 1 | LTA(C804A(Thr26Asn)) | 59 | 23 | Thrombospondin4(G1186C(Ala387Pro)) | 0.147 | 9.7 | 3.1 |
| 8 | 3 | TNFalfa(G-238A) | 58 | 1 | LTA(C804A(Thr26Asn)) | 0.147 | 9.4 | 3.1 |
| 35 | 23 | beta Fib(C148T) | 57 | 3 | LTA(A252G) | 0.039 | 7.3 | 3.1 |
| 26 | 23 | HANP(T2238C) | 58 | 1 | LTA(C804A(Thr26Asn)) | 0.145 | 9.2 | 3 |
| 14 | 23 | CRP(G1059C) | 58 | 1 | LTA(C804A(Thr26Asn)) | 0.144 | 9.1 | 3 |
| 53 | 1 | apolipoproteinE(E3 | 58 | 1 | LTA(C804A(Thr26Asn)) | 0.137 | 8.2 | 3 |
| | | inexon 4(Arg 158Cys) | | | | | | |
| 21 | 1 | AT2-receptor | 72 | 1 | PGC-1(G1302A(Thr394Thr)) | 0.044 | 7.9 | 3 |
| 20 | 1 | ACE | 44 | 23 | microsomal triglyceride transfer | 0.043 | 7.5 | 3 |
| | | | | | protein(G-493T) | | | |

The polymorphism number in Table 9-1 and 9-2 represents genetic polymorphisms in table 10-1 and table 10-2. The polymorphism numbers 1-49 are in common with polymorphism numbers extracted in Example 4. The polymorphism numbers 50-72 are newly extracted genetic polymorphisms.

TABLE 10-1

| Polymorphism | | Name of Genetic Polymorphism | | |
|---|---|---|---|---|
| No. | Symbol | Name of Gene | site | ref. SNP ID |
| 1 | N1 | e NOS | T-786C + 4repeat | rs2070744 |
| 2 | NOS3 | e NOS | G894T(Glu298Asp) | rs1799983 |
| 6 | IL62 | Interleukin-6 | C-634G | rs1800796 |
| 7 | N10 | TGF -beta 1 | T29C(Leu10Pro) | 329th of AY330201 |
| 8 | TNFa2 | Tumor necrosis factor- α | G-308A | rs1800629 |
| 10 | MMP12 | MMP-12 | A-82G | rs2276109 |
| 11 | MCP1 | MCP-1(chemokine) | A-2518G | rs1024611 |
| 14 | CRP1 | C-reactive protein | G1059C | rs1800947 |
| 16 | ESL2 | E-selectin | A561C(Ser128Arg) | rs5361 |
| 17 | ICAM1 | intercellular adhesin molecule 1 | G/A(E469K) | rs5498 |
| 18 | ESL1 | E-selectin | G98T | rs1805193 |
| 20 | ACE ID | ACE | I/D type | 1451 to 1738th |
| | | | | deletion of X62855 |
| 21 | AGTR1-3 | AT1 receptor | A1166C | rs5186 |
| 24 | β 2AR4 | β 2-Adrenergic Receptor | C79G | rs1042714 |
| 25 | β 2AR-1 | β 2-Adrenergic Receptor | A46G(Arg16Gly) | rs1042713 |
| 26 | HANP1 | Human Atrial Natriuuretic | T2238C | rs5065 |
| 27 | GP3A | GP IIB IIIa | C1565T (PIA2) | rs5918 |
| 29 | GP1ba | Human Platelet Antigen-2 | C1018T(Thr145Met) | rs6065 |
| 35 | FGB3 | beta Fibrinogen | C148T | rs1800787 |
| 36 | PAI | PAI-I | 4G/5G at -668 | rs1799889 |
| 37 | MTHFR | MTHFR | C677T | rs1801133 |
| 38 | ABCA1 | ABCA 1 | G1051A(Arg219Lys) | rs2230806 |
| 40 | PONA1 | PON1 | A584G(Gln192Arg) | rs662, (2003/8/7 |
| | | | | site correction 575→ |
| | | | | 584) |
| 41 | PAR2 | PPAR gamma | C/G(Pro12Ala) | rs1801282 |
| 42 | HL1 | hepatic lipase | C-480T | rs1800588 |
| 44 | MTP1 | microsomal trigyceride transfer | G-493T | rs1800591 |
| | | protein | | |
| 47 | S2AR | serotonin 2A receptor | T102C | rs6313 |
| 49 | MMP71 | matrilysin promoter | A-181G | 1022th of L22525 |
| 50 | N7 | p 22phox | C242T(His72Tyr) | rs4673 |
| 51 | CF12 | Factor XII | C46T(Arg 353Gln) | rs1801020 |
| 52 | GP1a1 | Glycoprotien I a | C807T | rs1126643 |
| 53 | APE3 | Apolipoprotein E ε 3 in exon 4 | C/T(Arg158Cy) | rs7412 |
| 54 | 1A2 | Glycoprotien I a | G873A | rs1062535 |
| 55 | BKR1 | bradykinin B2receptor | C-58T | rs1799722 |
| 56 | REG1 | Resistin | ATG 6 | rs3833230 |
| | | | repeat(1:6/6, 2:6/7, | |
| | | | 3:7/7, 4:7/8, 5:8/8) | |
| 57 | LTA1 | Lymphotoxin-alfa | A252G | 1069th of M16441 |
| 58 | LTA2 | Lymphotoxin-alfa | C804A(Thr26Asn) | rs1041981 |
| 59 | TS41 | Thrombospondin-4 | G1186C(Ala387Pro) | rs1866389 |
| 60 | APM12 | ADIPONECTIN | G276T | rs1501299, IMS- |
| | | | | JST013728 |

TABLE 10-2

| Polymorphism | | Name of Genetic Polymorphism | | |
|---|---|---|---|---|
| No. | Symbol | Name of Gene | site | ref. SNP ID |
| 61 | RAGE3 | RAGE | G/A(Gly82Ser ) | rs2070600 |
| 62 | TS11 | Thrombospondin-1 | A2210G(Asn700Ser | 55322th of AC037198 |
| 63 | 2C9-3 | CYP2C9*3 | A1075C(Leu359Ile) | rs1057910 |
| 64 | IL1B2 | IL-1 β | C3953T | rs1143634 |
| 65 | IL-181 | Interleukin-18 | C-607A | rs1946518 |
| 66 | MS1 | Methionine synthase | A2756G(Asp919Gly) | rs1805087 |
| 67 | VWF2 | von Willebrand Factor | G-1051A | rs7965413 |
| 68 | IL102 | Interleukin-10 | C-819T | rs1800871 |
| 69 | IL-182 | Interleukin-18 | G-137C | rs187238 |
| 70 | TPO1 | Thrombopoietin | A5713G | rs6141 |
| 71 | LRP1 | LDL receptor related protein | C766T | 516th of AF058399 |
| 72 | PGC11 | Peroxisome proliferation | G1302A(Thr394Thr) | rs2970847 |
| | | activated receptor γ | | |
| | | coactivator-1(PGC-1) | | |

In Example 5, wider range of genetic polymorphisms was searched. Thus, even in a combination of two genetic polymorphisms, 13 sets of genetic polymorphisms which show an odds ratio of 10 or more (16 genetic polymorphisms relate to these 13 sets), 12 sets of genetic polymorphisms which show an odds ratio of 4 or more to less than 10 (12 genetic polymorphisms relate to these 12 sets except the 16 genetic polymorphisms), and 39 sets of genetic polymorphisms which show an odds ratio of 3 or more to less than 4 (22 genetic polymorphisms relate to these 39 sets except the 16+2 genetic polymorphisms.) were extracted.

When the subject having one or more of these sets of genetic polymorphisms is determined as one having a risk, 335 cases (82.7%) of 405 cases with early arteriosclerosis could be explained by these sets of genetic polymorphisms. In contrast, only 102 cases (27.8%) of 367 cases with non-early arteriosclerosis cases had any of these sets of genetic polymorphisms and were determined as one having a risk. Specifically, if the subject in this population are examined on all genetic polymorphisms shown in table 10-1 and table 10-2 and the risk is determined based on whether or not the subject has any of sets of genetic polymorphisms shown in table 9-1 and table 9-2, the risk can be determined only based on genetic information. Thus, it was found that even when sets of genetic polymorphisms having a relatively low odds ratio, such as an odds ratio of 3 or more, were extracted and the number of combination of genetic polymorphisms are reduced to about two, the result of extraction is effective.

Compared the genetic polymorphisms extracted in the Example 5 (table 10-1 and table 10-2) with the genetic polymorphisms extracted in the Example 4 (table 2-1 and table 2-2), 28 genetic polymorphisms were extracted from the genetic polymorphisms shown in table 2 and the rest of the 23 were extracted from the genetic polymorphisms newly investigated in the present example. Thus, it was found that even when 50% or more of genetic polymorphisms shown in table 2-1 and table 2-2 (or table 10-1 and table 10-2) were used and newly extracted genetic polymorphisms were added, the risk could be accurately determined.

Next, 5 genetic polymorphisms (the polymorphism Nos. 49, 25, 29, 2, and 56) were randomly removed from the genetic polymorphisms shown in table 10-1 and table 10-2 and the risk was determined in the same way. 331 cases (81.7%) of 405 cases with early arteriosclerosis could be explained by these sets of genetic polymorphisms. In contrast, only 102 cases (27.8%) of 367 cases with non-early arteriosclerosis had any of these sets of genetic polymorphisms and determined as having a risk. Also, another 5 genetic polymorphisms (the polymorphism Nos. 62, 57, 58, 65, and 36) were removed from the genetic polymorphisms shown in table 10-1 and table 10-2 and the risk was determined in the same way. 324 cases (80.0%) of 405 cases with early arteriosclerosis could be explained by these sets of genetic polymorphisms. In contrast, only 102 cases (27.8%) of 367 cases with non-early arteriosclerosis had any of these sets of genetic polymorphisms and determined as having a risk. Further, another 5 genetic polymorphisms (the polymorphism Nos. 54, 27, 63, 10, and 70) were removed from the genetic polymorphisms shown in table 10-1 and table 10-2 and the risk was determined in the same way. 326 cases (80.5%) of 405 cases with early arteriosclerosis could be explained by these sets of genetic polymorphisms. In contrast, only 102 cases (27.8%) of 367 cases with non-early arteriosclerosis had any of these sets of genetic polymorphisms and determined as having a risk. Thus, it was found that even when about 5 genetic polymorphisms were removed from the genetic polymorphisms shown in table 10-1 and table 10-2, the risk could be determined with certain accuracy. Based on the results of the tables 1 and 9, it is obvious that if the genetic polymorphisms in the sets of genetic polymorphisms having low odds and relatively low frequency of appearance are removed, the number of genetic polymorphisms to be examined can be reduced according to convenience without lowering the accuracy relatively.

According to the Examples 1 to 5, it was verified that the present invention enabled the determination of the risk of arteriosclerotic disease which cannot be carried out when odds of a single genetic polymorphism are accumulated, and further enabled the determination of the risk of arteriosclerotic disease with extreme accuracy only by examining about 50 genetic polymorphisms.

The present invention provides a method for determining the risk of arteriosclerotic disease which can accurately determine the tendency to develop an arteriosclerotic disease or tendency for the advance of the disease as the risk of arteriosclerotic disease and can be utilized in prevention and treatment of arteriosclerosis. In addition, the present invention provides a method for revealing an arteriosclerotic disease-associated factor, a method for measuring the risk of arteriosclerotic disease, a method for detecting genetic polymorphism, a genetic marker, a kit for analyzing genetic polymorphism, an array for determining the risk of arteriosclerotic disease, apparatus for determining the risk of arteriosclerotic disease and a program for determining the risk of arteriosclerotic disease which are used in, for example, determination of the risk.

1. A method for determining the risk of arteriosclerotic disease, comprising a risk evaluation process for evaluating the risk of arteriosclerosis caused by genetic polymorphisms, based on the risk of arteriosclerosis inherent to a combination of plural genetic polymorphisms, from the genotype of a subject on the genetic polymorphisms,
wherein the combination of plural genetic polymorphisms includes at least one combination of plural genetic polymorphisms having a significant positive correlation with the carotid arterial intima-media thickness.

2. A method for determining the risk of arteriosclerotic disease according to claim 1, wherein the risk of arteriosclerosis inherent to the combination of plural genetic polymorphisms is set in accordance with whether or not the combination has a significant positive correlation with the carotid arterial intima-media thickness.

3. A method for determining the risk of arteriosclerotic disease according to claim 1, wherein the risk of arteriosclerosis inherent to the combination of plural genetic polymorphisms is set in accordance with the odds ratio where the combination has a significant positive correlation with the carotid arterial intima-media thickness.

4. A method for determining the risk of arteriosclerotic disease according to claim 1, wherein the risk of arteriosclerosis inherent to the combination of plural genetic polymorphisms is set in accordance with an an amount of increase in the carotid arterial intima-media thickness.

5. A method for determining the risk of arteriosclerotic disease according to claim 1, wherein the combination of plural genetic polymorphisms comprises at least one set of arteriosclerosis-associated genetic polymorphisms selected from sets of arteriosclerosis-associated genetic polymorphisms shown in following Tables 9-1 and 9-2.
TABLE 9-1

| Poly- | | | Poly- | | | | | |
|---|---|---|---|---|---|---|---|---|
| morphism | | Name of Genetic | morphism | | Name of Genetic | | Chi- | Odds |
| No. | Category | Polymorphism | No. | Category | Polymorphism | Frequency | square | Ratio |
| 42 | 12 | hepatic_lipase | 61 | 1 | RAGE(Gly82Ser) | 0.016 | 11.8 | 99 |
| 41 | 1 | PPAR_gamma | 61 | 1 | RAGE(Gly82Ser) | 0.014 | 9.7 | 99 |
| 18 | 23 | E-selectin | 58 | 1 | LTA(C804A(Thr26Asn)) | 0.014 | 9.6 | 99 |
| 18 | 23 | E-selectin | 57 | 3 | LTA(A252G) | 0.013 | 9.4 | 99 |
| 61 | 1 | RAGE(Gly82Ser) | 70 | 12 | Thrombopoietin(A5713G) | 0.013 | 9 | 99 |
| 7 | 12 | TGF beta | 61 | 1 | RAGE(Gly82Ser) | 0.012 | 8.8 | 99 |
| 2 | 23 | Enos 298 | 11 | 1 | MCP-1(A-2518G) | 0.011 | 7.9 | 99 |
| 27 | 3 | GPIIbIIIa | 61 | 1 | RAGE(Gly82Ser) | 0.018 | 9.1 | 11.9 |
| 61 | 1 | RAGE(Gly82Ser) | 63 | 12 | CYP2C9_3(Leu359Ile) | 0.018 | 9 | 11.8 |
| 61 | 1 | RAGE(Gly82Ser) | 68 | 23 | IL-10(C-819T) | 0.017 | 8.3 | 11.1 |
| 10 | 12 | MMP-12 | 61 | 1 | RAGE(Gly82Ser) | 0.016 | 8.1 | 10.8 |
| 47 | 12 | serotonin_2A_receptor | 61 | 1 | RAGE(Gly82Ser) | 0.016 | 8 | 10.8 |
| 6 | 12 | interleukin6(C-634G) | 61 | 1 | RAGE(Gly82Ser) | 0.015 | 7.3 | 10 |
| 51 | 23 | FactorXII | 61 | 1 | RAGE(Gly82Ser) | 0.015 | 7.1 | 9.8 |
| 38 | 3 | ABCA1 | 20 | 1 | ACE | 0.02 | 7.6 | 6.3 |
| 20 | 1 | ACE | 52 | 3 | glycoproteinIa(C807T) | 0.02 | 7.5 | 6.3 |
| 37 | 3 | MTHFR(C677T) | 58 | 1 | LTA(C804A(Thr26Asn)) | 0.029 | 10.4 | 6 |
| 20 | 1 | ACE | 54 | 1 | GP Ia(G873A) | 0.019 | 7 | 6 |
| 37 | 3 | MTHFR(C677T) | 57 | 3 | LTA(A252G) | 0.027 | 9.2 | 5.5 |
| 1 | 23 | Enos786 | 57 | 3 | LTA(A252G) | 0.134 | 12.7 | 4.4 |
| 60 | 3 | Adiponectin(G276T) | 66 | 23 | Methionine | 0.03 | 8.3 | 4.4 |
| | | | | | synthase(A2756G(Asp919gly)) | | | |
| 36 | 12 | PAI-1 | 62 | 1 | Thrombospondin-1(A2210G) | 0.482 | 88.9 | 4.3 |
| 57 | 3 | LTA(A252G) | 69 | 3 | IL-18(G-137C) | 0.111 | 10.4 | 4.3 |
| 58 | 1 | LTA(C804A(Thr26Asn)) | 69 | 3 | IL-18(G-137C) | 0.111 | 10.4 | 4.3 |
| 7 | 12 | TGF beta | 62 | 1 | Thrombospondin-1(A2210G) | 0.395 | 76.9 | 4 |
| 49 | 1 | matrilyn promoter(A-181G) | 62 | 1 | Thrombospondin-1(A2210G) | 0.488 | 79 | 3.9 |
| 62 | 1 | Thrombospondin-1(A2210G) | 71 | 1 | LDL receptor related | 0.451 | 77.9 | 3.9 |
| | | | | | protein(C766T) | | | |
| 21 | 1 | AT2-receptor | 62 | 1 | Thrombospondin-1(A2210G) | 0.482 | 77 | 3.8 |
| 62 | 1 | Thrombospondin-1(A2210G) | 68 | 23 | IL-10(C-819T) | 0.492 | 76.7 | 3.8 |
| 55 | 12 | bradykinin B2 | 62 | 1 | Thrombospondin-1(A2210G) | 0.381 | 68.1 | 3.8 |
| | | receptor(C-58T) | | | | | | |
| 57 | 3 | LTA(A252G) | 64 | 1 | interleukin 1 beta(C3953T) | 0.139 | 11.5 | 3.8 |

TABLE 9-2

| Poly- | | | Poly- | | | | | |
|---|---|---|---|---|---|---|---|---|
| morphism | | Name of Genetic | morphism | | | | Chi- | Odds |
| No. | Category | Polymorphism | No. | Category | Name of Genetic Polymorphism | Frequency | square | Ratio |
| 50 | 1 | p22phox | 62 | 1 | Thrombospondin-1(A2210G) | 0.481 | 73.6 | 3.7 |
| 24 | 1 | beta2 | 62 | 1 | Thrombospondin-1(A2210G) | 0.482 | 72.6 | 3.7 |
| | | Adrenoreceptor(C79T) | | | | | | |
| 53 | 1 | apolipoproteinE(E3 | 57 | 3 | LTA(A252G) | 0.14 | 10.7 | 3.7 |
| | | inexon 4(Arg 158Cys) | | | | | | |
| 51 | 23 | FactorXII | 62 | 1 | Thrombospondin-1(A2210G) | 0.499 | 72.8 | 3.6 |
| 62 | 1 | Thrombospondin-1(A2210G) | 65 | 12 | IL-18(C-607A) | 0.439 | 68.2 | 3.6 |
| 8 | 3 | TNFalfa(G-238A) | 57 | 3 | LTA(A252G) | 0.149 | 11.6 | 3.6 |
| 57 | 3 | LTA(A252G) | 59 | 23 | Thrombospondin4(G1186C(Ala387Pro)) | 0.147 | 11.4 | 3.6 |
| 1 | 23 | Enos786 | 58 | 1 | LTA(C804A(Thr26Asn)) | 0.135 | 10.6 | 3.6 |
| 68 | 23 | IL-10(C-819T) | 72 | 1 | PGC-1(G1302A(Thr394Thr)) | 0.044 | 9.9 | 3.6 |
| 29 | 23 | HPA-2(Thr145Met) | 40 | 1 | PON1(Gly192Arg) | 0.031 | 7 | 3.6 |
| 42 | 12 | hepatic_lipase | 62 | 1 | Thrombospondin-1(A2210G) | 0.425 | 67 | 3.5 |
| 17 | 12 | ICAM1(E469K) | 62 | 1 | Thrombospondin-1(A2210G) | 0.457 | 66.4 | 3.5 |
| 47 | 12 | serotonin_2A_receptor | 62 | 1 | Thrombospondin-1(A2210G) | 0.401 | 64 | 3.5 |
| 14 | 23 | CRP(G1059C) | 57 | 3 | LTA(A252G) | 0.145 | 11.2 | 3.5 |
| 41 | 1 | PPAR_gamma | 72 | 1 | PGC-1(G1302A(Thr394Thr)) | 0.043 | 9.2 | 3.5 |
| 6 | 12 | interleukin6(C-634G) | 62 | 1 | Thrombospondin-1(A2210G) | 0.493 | 64.2 | 3.4 |
| 25 | 12 | beta-adrenergic | 62 | 1 | Thrombospondin-1(A2210G) | 0.42 | 64 | 3.4 |
| | | receptor(A46G) | | | | | | |
| 57 | 3 | LTA(A252G) | 65 | 12 | IL-18(C-607A) | 0.076 | 16 | 3.4 |
| 26 | 23 | HANP(T2238C) | 57 | 3 | LTA(A252G) | 0.143 | 10.7 | 3.4 |
| 62 | 1 | Thrombospondin-1(A2210G) | 67 | 23 | von Willebrand Factor (G-1051A) | 0.415 | 56.9 | 3.2 |
| 58 | 1 | LTA(C804A(Thr26Asn)) | 65 | 12 | IL-18(C-607A) | 0.078 | 15.1 | 3.2 |
| 58 | 1 | LTA(C804A(Thr26Asn)) | 64 | 1 | interleukin 1 beta(C3953T) | 0.137 | 9.2 | 3.2 |
| 56 | 3 | resistin(ATG repeat) | 57 | 3 | LTA(A252G) | 0.137 | 9 | 3.2 |
| 36 | 12 | PAI-1 | 72 | 1 | PGC-1(G1302A(Thr394Thr)) | 0.041 | 7.9 | 3.2 |
| 35 | 23 | beta Fib(C148T) | 58 | 1 | LTA(C804A(Thr26Asn)) | 0.039 | 7.5 | 3.2 |
| 58 | 1 | LTA(C804A(Thr26Asn)) | 59 | 23 | Thrombospondin4(G1186C(Ala387Pro)) | 0.147 | 9.7 | 3.1 |
| 8 | 3 | TNFalfa(G-238A) | 58 | 1 | LTA(C804A(Thr26Asn)) | 0.147 | 9.4 | 3.1 |
| 35 | 23 | beta Fib(C148T) | 57 | 3 | LTA(A252G) | 0.039 | 7.3 | 3.1 |
| 26 | 23 | HANP(T2238C) | 58 | 1 | LTA(C804A(Thr26Asn)) | 0.145 | 9.2 | 3 |
| 14 | 23 | CRP(G1059C) | 58 | 1 | LTA(C804A(Thr26Asn)) | 0.144 | 9.1 | 3 |
| 53 | 1 | apolipoproteinE(E3 | 58 | 1 | LTA(C804A(Thr26Asn)) | 0.137 | 8.2 | 3 |
| | | inexon 4(Arg 158Cys) | | | | | | |
| 21 | 1 | AT2-receptor | 72 | 1 | PGC-1(G1302A(Thr394Thr)) | 0.044 | 7.9 | 3 |
| 20 | 1 | ACE | 44 | 23 | microsomal triglyceride transfer | 0.043 | 7.5 | 3 |
| | | | | | protein(G-493T) | | | |

In Tables 9-1 and 9-2, “Polymorphism No.” represents a genetic polymorphism having the same number in following Tables 10; the number of “Category” represents a genotype constituting the combination among genotypes of genetic polymorphisms, in which “1” represents homozygosis of the polymorphism having an anterior base in alphabetic order of substituted bases of genetic polymorphism; “2” represents heterozygosis of the polymorphism;
“3” represents homozygosis of the polymorphism having a posterior base in alphabetic order of substituted bases of genetic polymorphism;
“1 2” represents a genotype as a collection of 1 and 2; and “2 3” represents a genotype as a collection of 10 and 3 in the sites of genetic polymorphisms shown in Tables 10-1 and 10-2.
TABLE 10-1

| Polymorphism | | Name of Genetic Polymorphism | | |
|---|---|---|---|---|
| No. | Symbol | Name of Gene | site | ref. SNP ID |
| 1 | N1 | e NOS | T-786C + 4repeat | rs2070744 |
| 2 | NOS3 | e NOS | G894T(Glu298Asp) | rs1799983 |
| 6 | IL62 | Interieukin-6 | C-634G | rs1800796 |
| 7 | N10 | TGF -beta 1 | T29C(Leu10Pro) | 329th of AY330201 |
| 8 | TNFa2 | Tumor necrosis factor- α | G-308A | rs1800629 |
| 10 | MMP12 | MMP-12 | A-82G | rs2276109 |
| 11 | MCP1 | MCP-1(chemokine) | A-2518G | rs1024611 |
| 14 | CRP1 | C-reactive protein | G1059C | rs1800947 |
| 16 | ESL2 | E-selectin | A561C(Ser128Arg) | rs5361 |
| 17 | ICAM1 | intercellular adhesin molecule 1 | G/A(E469K) | rs5498 |
| 18 | ESL1 | E-selectin | G98T | rs1805193 |
| 20 | ACE ID | ACE | I/D type | 1451 to 1738th |
| | | | | deletion of X62855 |
| 21 | AGTR1-3 | AT1 receptor | A1166C | rs5186 |
| 24 | β 2AR4 | β 2-Adrenergic Receptor | C79G | rs1042714 |
| 25 | β 2AR-1 | β 2-Adrenergic Receptor | A46G(Arg16Gly) | rs1042713 |
| 26 | HANP1 | Human Atrial Natriuuretic | T2238C | rs5065 |
| 27 | GP3A | GP IIB IIIa | C1565T (PIA2) | rs5918 |
| 29 | GP1ba | Human Platelet Antigen-2 | C1018T(Thr145Met) | rs6065 |
| 35 | FGB3 | beta Fibrinogen | C148T | rs1800787 |
| 36 | PAI | PAI-I | 4G/5G at -668 | rs1799889 |
| 37 | MTHFR | MTHFR | C677T | rs1801133 |
| 38 | ABCA1 | ABCA1 | G1051A(Arg219Lys) | rs2230806 |
| 40 | PONA1 | PON1 | A584G(Gln192Arg) | rs662, (2003/8/7 |
| | | | | site correction 575→ |
| | | | | 584) |
| 41 | PAR2 | PPAR gamma | C/G(Pro12Ala) | rs1801282 |
| 42 | HL1 | hepatic lipase | C-480T | rs1800588 |
| 44 | MTP1 | microsomal trigyceride transfer | G-493T | rs1800591 |
| | | protein | | |
| 47 | S2AR | serotonin 2A receptor | T102C | rs6313 |
| 49 | MMP71 | matrilysin promoter | A-181G | 1022th of L22525 |
| 50 | N7 | p 22phox | C242T(His72Tyr) | rs4673 |
| 51 | CF12 | Factor XII | C46T(Arg 353Gln) | rs1801020 |
| 52 | GP1a1 | Glycoprotien I a | C807T | rs1126643 |
| 53 | APE3 | Apolipoprotein E ε 3 in exon 4 | C/T(Arg158Cy) | rs7412 |
| 54 | 1A2 | Glycoprotien I a | G873A | rs1062535 |
| 55 | BKR1 | bradykinin B2receptor | C-58T | rs1799722 |
| 56 | REG1 | Resistin | ATG 6 | rs3833230 |
| | | | repeat(1:6/6, 2:6/7, | |
| | | | 3:7/7, 4:7/8, 5:8/8) | |
| 57 | LTA1 | Lymphotoxin-alfa | A252G | 1069th of M16441 |
| 58 | LTA2 | Lymphotoxin-alfa | C804A(Thr26Asn) | rs1041981 |
| 59 | TS41 | Thrombospondin-4 | G1186C(Ala387Pro) | rs1866389 |
| 60 | APM12 | ADIPONECTIN | G276T | rs1501299, IMS- |
| | | | | JST013728 |

TABLE 10-2

| Polymorphism | | Name of Genetic Polymorphism | | |
|---|---|---|---|---|
| No. | Symbol | Name of Gene | site | ref. SNP ID |
| 61 | RAGE3 | RAGE | G/A(Gly82Ser ) | rs2070600 |
| 62 | TS11 | Thrombospondin-1 | A2210G(Asn700Ser) | 55322th of AC037198 |
| 63 | 2C9-3 | CYP2C9*3 | A1075C(Leu359Ile) | rs1057910 |
| 64 | IL1B2 | IL-1 β | C3953T | rs1143634 |
| 65 | IL-181 | Interleukin-18 | C-607A | rs1946518 |
| 66 | MS1 | Methionine synthase | A2756G(Asp919Gly) | rs1805087 |
| 67 | VWF2 | von Willebrand Factor | G-1051A | rs7965413 |
| 68 | IL102 | Interleukin-10 | C-819T | rs1800871 |
| 69 | IL-182 | Interleukin-18 | G-137C | rs187238 |
| 70 | TPO1 | Thrombopoietin | A5713G | rs6141 |
| 71 | LRP1 | LDL receptor related protein | C766T | 516th of AF058399 |
| 72 | PGC11 | Peroxisome proliferation | G1302A(Thr394Thr) | rs2970847 |
| | | activated receptor γ | | |
| | | coactivator-1(PGC-1) | | |

6. A method for determining the risk of arteriosclerotic disease according to claim 5, wherein at least 50% of the genetic polymorphisms shown in Table 10-1 and table 10-2, are used.

7. A method for determining the risk of arteriosclerotic disease according to claim 5, wherein at least 90% of the genetic polymorphisms shown in Table 10-1 and table 10-2, are used.

8. A method for determining the risk of arteriosclerotic disease according to claim 6, wherein the combination of plural genetic polymorphisms further includes at least one set of arteriosclerosis-associated genetic polymorphisms selected from sets of arteriosclerosis-associated genetic polymorphisms shown in following Tables 1-1, 1-2, 1-3 and 1-4.
TABLE 1-1

| Polymorphism | | Polymorphism | | Polymorphism | | Polymorphism | |
|---|---|---|---|---|---|---|---|
| No. | Category | No. | Category | No. | Category | No. | Category |
| 3 | 12 | 46 | 12 | | | | |
| 1 | 3 | 21 | 1 | 28 | 12 | | |
| 1 | 3 | 28 | 12 | 29 | 1 | | |
| 1 | 3 | 28 | 12 | 40 | 12 | | |
| 1 | 3 | 28 | 12 | 34 | 23 | | |
| 3 | 12 | 4 | 1 | 7 | 23 | | |
| 3 | 12 | 7 | 23 | 17 | 12 | | |
| 3 | 12 | 7 | 23 | 31 | 12 | | |
| 3 | 12 | 7 | 23 | 34 | 12 | | |
| 3 | 12 | 8 | 3 | 46 | 12 | | |
| 3 | 12 | 9 | 12 | 46 | 12 | | |
| 3 | 12 | 12 | 23 | 31 | 12 | | |
| 3 | 12 | 12 | 23 | 46 | 12 | | |
| 3 | 12 | 13 | 23 | 46 | 12 | | |
| 3 | 12 | 14 | 23 | 46 | 12 | | |
| 3 | 12 | 15 | 12 | 25 | 12 | | |
| 3 | 12 | 15 | 12 | 46 | 12 | | |
| 3 | 12 | 22 | 12 | 31 | 12 | | |
| 3 | 12 | 24 | 12 | 46 | 12 | | |
| 3 | 12 | 25 | 12 | 31 | 12 | | |
| 3 | 12 | 26 | 3 | 46 | 12 | | |
| 3 | 12 | 28 | 23 | 31 | 12 | | |
| 3 | 12 | 28 | 23 | 46 | 12 | | |
| 3 | 12 | 29 | 12 | 46 | 12 | | |
| 3 | 12 | 30 | 12 | 46 | 12 | | |
| 3 | 12 | 31 | 12 | 41 | 1 | | |
| 3 | 12 | 41 | 1 | 42 | 12 | | |
| 3 | 12 | 41 | 1 | 46 | 12 | | |
| 3 | 12 | 43 | 23 | 46 | 12 | | |
| 3 | 12 | 44 | 12 | 46 | 12 | | |
| 3 | 12 | 42 | 12 | 47 | 23 | | |
| 3 | 12 | 46 | 12 | 49 | 12 | | |
| 4 | 1 | 7 | 12 | 28 | 12 | | |
| 4 | 1 | 28 | 12 | 40 | 12 | | |
| 4 | 23 | 25 | 1 | 40 | 12 | | |
| 6 | 1 | 7 | 1 | 20 | 1 | | |
| 7 | 1 | 37 | 3 | 45 | 1 | | |
| 11 | 3 | 20 | 3 | 32 | 12 | | |
| 11 | 1 | 35 | 1 | 38 | 3 | | |
| 11 | 1 | 38 | 3 | 40 | 12 | | |
| 17 | 1 | 36 | 12 | 37 | 3 | | |
| 17 | 1 | 37 | 3 | 48 | 1 | | |
| 17 | 23 | 28 | 12 | 40 | 12 | | |
| 20 | 3 | 22 | 1 | 24 | 23 | | |

TABLE 1-2

| Polymorphism | | Polymorphism | | Polymorphism | | Polymorphism | |
|---|---|---|---|---|---|---|---|
| No. | Category | No. | Category | No. | Category | No. | Category |
| 20 | 3 | 24 | 23 | 33 | 12 | | |
| 20 | 3 | 24 | 23 | 38 | 12 | | |
| 20 | 3 | 24 | 23 | 48 | 1 | | |
| 17 | 1 | 20 | 3 | 32 | 12 | | |
| 20 | 3 | 32 | 12 | 40 | 12 | | |
| 20 | 3 | 32 | 12 | 38 | 3 | | |
| 21 | 1 | 28 | 12 | 40 | 12 | | |
| 11 | 12 | 25 | 1 | 38 | 3 | | |
| 17 | 1 | 25 | 1 | 38 | 3 | | |
| 23 | 12 | 25 | 1 | 38 | 3 | | |
| 25 | 1 | 36 | 1 | 38 | 3 | | |
| 25 | 1 | 38 | 3 | 40 | 12 | | |
| 28 | 12 | 32 | 3 | 40 | 12 | | |
| 7 | 12 | 28 | 12 | 33 | 12 | | |
| 28 | 12 | 38 | 23 | 40 | 12 | | |
| 31 | 3 | 32 | 3 | 47 | 1 | | |
| 4 | 12 | 31 | 3 | 45 | 23 | | |
| 15 | 1 | 31 | 3 | 45 | 23 | | |
| 31 | 3 | 45 | 23 | 46 | 12 | | |
| 34 | 3 | 36 | 12 | 39 | 12 | | |
| 34 | 3 | 37 | 1 | 47 | 1 | | |
| 17 | 12 | 34 | 3 | 39 | 12 | | |
| 34 | 3 | 39 | 12 | 41 | 1 | | |
| 34 | 3 | 39 | 12 | 45 | 1 | | |
| 16 | 1 | 34 | 1 | 40 | 1 | | |
| 18 | 1 | 34 | 1 | 40 | 1 | | |
| 24 | 1 | 34 | 1 | 40 | 1 | | |
| 34 | 1 | 40 | 1 | 45 | 1 | | |
| 12 | 12 | 39 | 12 | 43 | 3 | | |
| 1 | 23 | 11 | 3 | 31 | 23 | 39 | 12 |
| 2 | 1 | 7 | 3 | 20 | 3 | 38 | 3 |
| 2 | 1 | 25 | 12 | 38 | 3 | 39 | 12 |
| 2 | 1 | 33 | 23 | 38 | 3 | 39 | 12 |
| 2 | 1 | 11 | 3 | 31 | 23 | 39 | 12 |
| 2 | 1 | 35 | 1 | 38 | 3 | 39 | 12 |
| 4 | 23 | 20 | 3 | 33 | 12 | 40 | 12 |
| 4 | 23 | 23 | 3 | 33 | 12 | 40 | 12 |
| 4 | 23 | 33 | 12 | 40 | 12 | 47 | 12 |
| 4 | 23 | 11 | 3 | 20 | 3 | 40 | 12 |
| 4 | 23 | 20 | 3 | 31 | 23 | 40 | 12 |
| 4 | 23 | 29 | 1 | 38 | 3 | 40 | 12 |
| 5 | 1 | 11 | 3 | 39 | 12 | 43 | 3 |
| 5 | 1 | 12 | 12 | 20 | 3 | 23 | 3 |
| 5 | 1 | 15 | 1 | 20 | 3 | 32 | 12 |

TABLE 1-3

| Polymorphism | | Polymorphism | | Polymorphism | | Polymorphism | |
|---|---|---|---|---|---|---|---|
| No. | Category | No. | Category | No. | Category | No. | Category |
| 5 | 1 | 20 | 3 | 23 | 3 | 31 | 3 |
| 5 | 1 | 20 | 3 | 37 | 23 | 39 | 12 |
| 5 | 1 | 12 | 12 | 25 | 1 | 44 | 1 |
| 5 | 1 | 25 | 1 | 39 | 12 | 45 | 1 |
| 6 | 12 | 11 | 3 | 37 | 23 | 39 | 12 |
| 6 | 12 | 11 | 3 | 39 | 12 | 43 | 3 |
| 6 | 1 | 15 | 1 | 17 | 3 | 25 | 1 |
| 6 | 1 | 17 | 3 | 33 | 12 | 40 | 12 |
| 6 | 1 | 11 | 3 | 20 | 1 | 42 | 12 |
| 6 | 1 | 20 | 1 | 23 | 3 | 40 | 12 |
| 6 | 23 | 9 | 1 | 22 | 23 | 25 | 1 |
| 6 | 23 | 22 | 23 | 25 | 1 | 42 | 12 |
| 6 | 23 | 20 | 3 | 25 | 1 | 46 | 12 |
| 7 | 3 | 9 | 12 | 20 | 3 | 38 | 3 |
| 7 | 3 | 13 | 3 | 20 | 3 | 38 | 3 |
| 7 | 3 | 14 | 3 | 20 | 3 | 38 | 3 |
| 7 | 3 | 20 | 3 | 22 | 12 | 38 | 3 |
| 7 | 3 | 20 | 3 | 23 | 3 | 38 | 3 |
| 7 | 3 | 20 | 3 | 27 | 3 | 38 | 3 |
| 7 | 3 | 20 | 3 | 29 | 1 | 38 | 3 |
| 7 | 3 | 20 | 3 | 30 | 1 | 38 | 3 |
| 7 | 3 | 20 | 3 | 33 | 12 | 40 | 12 |
| 7 | 3 | 20 | 3 | 35 | 1 | 38 | 3 |
| 7 | 3 | 20 | 3 | 38 | 3 | 49 | 12 |
| 7 | 23 | 17 | 23 | 33 | 1 | 40 | 12 |
| 9 | 1 | 12 | 12 | 34 | 3 | 36 | 12 |
| 9 | 1 | 20 | 3 | 31 | 3 | 36 | 12 |
| 9 | 1 | 31 | 3 | 33 | 12 | 40 | 12 |
| 9 | 1 | 12 | 12 | 24 | 1 | 34 | 3 |
| 9 | 12 | 25 | 1 | 39 | 12 | 45 | 1 |
| 9 | 12 | 25 | 1 | 39 | 12 | 46 | 12 |
| 11 | 3 | 16 | 1 | 31 | 23 | 39 | 12 |
| 11 | 3 | 17 | 12 | 31 | 23 | 39 | 12 |
| 11 | 3 | 18 | 1 | 31 | 23 | 39 | 12 |
| 11 | 3 | 22 | 12 | 31 | 23 | 39 | 12 |
| 11 | 3 | 23 | 23 | 31 | 23 | 39 | 12 |
| 11 | 3 | 31 | 23 | 36 | 12 | 39 | 12 |
| 11 | 3 | 39 | 12 | 42 | 12 | 43 | 3 |
| 11 | 3 | 39 | 12 | 43 | 3 | 47 | 12 |
| 12 | 12 | 20 | 3 | 23 | 3 | 36 | 12 |
| 12 | 12 | 23 | 3 | 32 | 12 | 41 | 1 |
| 12 | 12 | 40 | 12 | 42 | 1 | 47 | 12 |
| 12 | 12 | 25 | 1 | 34 | 12 | 44 | 1 |
| 13 | 3 | 25 | 12 | 38 | 3 | 39 | 12 |

TABLE 1-4

| Polymorphism | | Polymorphism | | Polymorphism | | Polymorphism | |
|---|---|---|---|---|---|---|---|
| No. | Category | No. | Category | No. | Category | No. | Category |
| 13 | 3 | 36 | 12 | 38 | 3 | 39 | 12 |
| 13 | 3 | 38 | 3 | 39 | 12 | 43 | 3 |
| 14 | 3 | 15 | 1 | 20 | 3 | 32 | 12 |
| 14 | 3 | 20 | 3 | 23 | 3 | 31 | 3 |
| 15 | 1 | 25 | 1 | 31 | 3 | 32 | 3 |
| 15 | 1 | 17 | 23 | 31 | 3 | 40 | 12 |
| 15 | 1 | 31 | 3 | 33 | 12 | 40 | 12 |
| 15 | 1 | 20 | 3 | 23 | 3 | 32 | 12 |
| 15 | 1 | 20 | 3 | 32 | 12 | 36 | 12 |
| 17 | 1 | 20 | 3 | 25 | 1 | 31 | 23 |
| 20 | 3 | 23 | 3 | 31 | 3 | 32 | 3 |
| 20 | 3 | 23 | 3 | 31 | 3 | 38 | 23 |
| 20 | 23 | 28 | 12 | 40 | 12 | 42 | 23 |
| 20 | 3 | 28 | 12 | 29 | 1 | 46 | 23 |
| 20 | 3 | 29 | 1 | 37 | 23 | 39 | 12 |
| 20 | 3 | 29 | 1 | 39 | 12 | 42 | 12 |
| 20 | 3 | 29 | 1 | 39 | 12 | 43 | 3 |
| 20 | 3 | 22 | 1 | 23 | 3 | 32 | 12 |
| 20 | 3 | 36 | 12 | 37 | 23 | 39 | 12 |
| 20 | 3 | 36 | 12 | 39 | 12 | 45 | 1 |
| 20 | 3 | 38 | 3 | 39 | 12 | 43 | 3 |
| 20 | 3 | 23 | 3 | 32 | 12 | 46 | 12 |
| 20 | 3 | 37 | 23 | 39 | 12 | 46 | 12 |
| 20 | 3 | 39 | 12 | 42 | 12 | 46 | 12 |
| 20 | 3 | 39 | 12 | 45 | 1 | 46 | 12 |
| 20 | 3 | 39 | 12 | 46 | 12 | 47 | 12 |
| 20 | 3 | 37 | 23 | 39 | 12 | 48 | 1 |
| 23 | 3 | 37 | 3 | 38 | 3 | 48 | 1 |
| 23 | 3 | 31 | 23 | 39 | 12 | 47 | 12 |
| 23 | 3 | 37 | 23 | 39 | 12 | 47 | 12 |
| 25 | 1 | 34 | 12 | 38 | 3 | 44 | 1 |
| 25 | 1 | 33 | 23 | 39 | 12 | 45 | 1 |
| 25 | 1 | 39 | 12 | 45 | 1 | 47 | 12 |
| 25 | 1 | 33 | 23 | 39 | 12 | 46 | 12 |
| 28 | 12 | 29 | 1 | 33 | 12 | 40 | 12 |
| 28 | 12 | 29 | 1 | 40 | 12 | 42 | 23 |
| 28 | 12 | 34 | 23 | 40 | 12 | 42 | 23 |
| 28 | 12 | 33 | 12 | 40 | 12 | 48 | 1 |
| 28 | 12 | 40 | 12 | 42 | 23 | 48 | 1 |
| 31 | 3 | 32 | 3 | 33 | 12 | 37 | 23 |
| 35 | 1 | 38 | 3 | 39 | 12 | 43 | 3 |
| 35 | 1 | 37 | 3 | 44 | 23 | 48 | 1 |
| 35 | 1 | 39 | 12 | 43 | 3 | 44 | 23 |
| 36 | 1 | 37 | 3 | 40 | 12 | 42 | 23 |

In Tables 1-1, 1-2, 1-3 and 1-4, “Polymorphism No.” represents a genetic polymorphism having the same number in following Tables 2-1 and 2-2; the number of “Category” represents a genotype constituting the combination among genotypes of genetic polymorphisms, in which “1” represents homozygosis of the polymorphism having an anterior base in alphabetic order of substituted bases of genetic polymorphism; “2” represents heterozygosis of the polymorphism; “3” represents homozygosis of the polymorphism having a posterior base in alphabetic order of substituted bases of genetic polymorphism;
“1 2” represents a genotype as a collection of 1 and 2; and “2 3” represents a genotype as a collection of 2 and 3 in the names of genetic polymorphisms shown in Tables 2-1 and 2-2.
TABLE 2-1

| Polymorphism | | Name of Genetic Polymorphism | | | Reference |
|---|---|---|---|---|---|
| No. | Symbol | Name of Gene | site | ref. SNP ID | Number |
| 1 | N1 | e NOS | T-786C + 4repeat | rs2070744 | 1 |
| 2 | NOS3 | e NOS | G894T(Glu298Asp) | rs1799983 | |
| 3 | IRS1 | IRS-1 | G3494A(Gly971Arg) | rs1801278 | |
| 4 | GSY | glycogen synthase | A/G(M416V) | rs5447 | 2 |
| 5 | | p22phox | C242T | | 3 |
| 6 | IL62 | Interleukin-6 | C-634G | rs1800796 | |
| 7 | N10 | TGF -beta 1 | T29C(Leu10Pro) | 329th of | 4 |
| | | | | AY330201 | |
| 8 | TNFa2 | Tumor necrosis factor- α | G-308A | rs1800629 | 25 |
| 9 | MMP9 | MMP-9 = gelatinase B | C-1562T | rs3918242 | 5 |
| 10 | MMP12 | MMP-12 | A-82G | rs2276109 | 6 |
| 11 | MCP1 | MCP-1(chemokine) | A-2518G | rs1024611 | 7 |
| 12 | MPO | myeloperoxidase | G-463A | rs2333227 | 8 |
| 13 | FR1 | Flactalkine receptor CX3CR1 | G84635A(Val249Ile) | rs3732379 | |
| 14 | CRP1 | C-reactive protein | G1059C | rs1800947 | 26 |
| 15 | CX37 | connexin37(gap junction protein) | C1019T(Pro319Ser) | rs1764391 | 21 |
| 16 | ESL2 | E-selectin | A561C(Ser128Arg) | rs5361 | 9 |
| 17 | ICAM1 | intercellular adhesin molecule 1 | G/A(E469K) | rs5498 | |
| 18 | ESL1 | E-selectin | G98T | rs1805193 | 22 |
| 19 | DRD2 | Dopamine D2 receptor | C/G(Ser311Cys) | rs1801028 | |
| 20 | ACE ID | ACE | I/D type | 1451 to | 23 |
| | | | | 1738th | |
| | | | | deletion of | |
| | | | | X62855 | |
| 21 | AGTR1 -3 | AT1 receptor | A1166C | rs5186 | |
| 22 | AGT1 | Angiotensinogen | T704C(M235T) | JST050962, | 10 |
| | | | | rs699 | |
| 23 | 3AR | beta 3 adreno receptor | T/C(Trp64Arg) | rs4994 | 27 |
| 24 | β 2AR4 | β 2-Adrenergic Receptor | C79G | rs1042714 | |
| 25 | β 2AR-1 | β 2-Adrenergic Receptor | A46G(Arg16Gly) | rs1042713 | |
| 26 | HANP1 | Human Atrial Natriuuretic Peptide | T2238C | rs5065 | |
| 27 | GP3A | GP IIB IIIa | C1565T(PIA2) | rs5918 | 24 |
| 28 | G1A3 | GP Ia | A1648G | rs1801106 | 11 |
| 29 | GP1ba | Human Platelet Antigen-2 | C1018T(Thr145Met) | rs6065 | 28 |
| 30 | GP61 | Glycoprotein VI | T/C(Ser219Pro) | rs1613662 | |

TABLE 2-2

| Polymorphism | | Name of Genetic Polymorphism | | | Reference |
|---|---|---|---|---|---|
| No. | Symbol | Name of Gene | site | ref. SNP ID | Number |
| 31 | | Glycoprotien I a | C807T | | 12 |
| 32 | Tbm3 | thrombomodulin | G33A | 1487th of | 13 |
| | | | | M74564 | |
| 33 | | Factor XII | C46T | rs1801020 | |
| 34 | FGA1 | alpha fibrinogen | A/G(Thr312Ala) | rs6050 | 29 |
| 35 | FGB3 | beta Fibrinogen | C148T | rs1800787 | 14 |
| 36 | PAI | PAI-I | 4G/5G at -668 | rs1799889 | 15 |
| 37 | MTHFR | MTHFR | C677T | rs1801133 | |
| 38 | ABCA1 | ABCA 1 | G1051A(Arg219Lys) | rs2230806 | 16 |
| 39 | PONA2 | HUMPONA | A172T(Met55Leu) | rs3202100 | |
| 40 | PONA1 | PON1 | A584G(Gln192Arg) | rs662, | |
| | | | | (2003/8/7 | |
| | | | | site correction | |
| | | | | 575→584) | |
| 41 | PAR2 | PPAR gamma | C/G(Pro12Ala) | rs1801282 | |
| 42 | HL1 | hepatic lipase | C-480T | rs1800588 | 17 |
| 43 | APE2 | Apolipoprotein E | T/C(Cys112Arg) | rs429358 | 30 |
| 44 | MTP1 | microsomal trigyceride | G-493T | rs1800591 | 31 |
| | | transfer protein | | | |
| 45 | LPL3 | Lipoprotein lipase | C/G(Ser 447 STOP) | rs328 | |
| 46 | ESRa1 | alfa estrogen receptor | T/C(P vuII) | rs2234693 | 18 |
| 47 | S2AR | serotonin 2A receptor | T102C | rs6313 | 19 |
| 48 | GCLM1 | glutamate-cysteine ligase, | C588T | 2670th of | 32 |
| | | modifier subunit | | U72210 | |
| 49 | MMP71 | matrilysin promoter | A-181G | 1022th of | 20 |
| | | | | L22525 | |

9. A method for determining the risk of arteriosclerotic disease according to claim 1, wherein the combination of plural genetic polymorphisms includes at least one set of arteriosclerosis-associated genetic polymorphisms selected from sets of arterosclerosis-associated genetic polymorphisms shown in following Tables 1-1, 1-2, 1-3 and 1-4.
In Tables 1-1,1-2, 1-3 and 1-4, “Polymorphism No.” represents a genetic polymorphism having the same number in following Tables 2-1 and 2-2; the number of “Category” represents a genotype constituting the combination among genotypes of genetic polymorphisms, in which “1” represents homozygosis of the polymorphism having an anterior base in alphabetic order of substituted bases of genetic polymorphism; “2” represents heterozygosis of the polymorphism; “3” represents homozygosis of the polymorphism having a posterior base in alphabetic order of substituted bases of genetic polymorphism; “1 2” represents a genotype as a collection of 1 and 2; and “2 3” represents a genotype as a collection of 2 and 3 in the names of genetic polymorphisms shown in Tables 2-1 and 2-2.

10. A method for determining the risk of arteriosclerotic disease according to claim 9, wherein at least 50% of the genetic polymorphisms shown in Tables 2-1 and 2-2, are used.

11. A method for determining the risk of arteriosclerotic disease according to claim 1, wherein the combination of plural genetic polymorphisms is selected so that,
when cases having a carotid arterial intima-media thickness at least 0.2 mm larger than the average of carotid arterial intima-media thickness of healthy subjects are defined as arteriosclerotic disease cases and the other cases are defined as non-arteriosclerotic disease cases,
cases having a combination of plural genetic polymorphisms showing a significant positive correlation with the carotid arterial intima-media thickness occupy 30% or more of a population of arteriosclerotic disease cases comprising at least 150 cases, and cases having a combination of plural genetic polymorphisms showing a significant positive correlation with the carotid arterial intima-media thickness occupy 15% or less of a population of non-arteriosclerotic disease cases comprising at least 150 cases.

12. A method for determining the risk of arteriosclerotic disease according to claim 11, wherein the population of arteriosclerotic disease cases and the population of non-arteriosclerotic disease cases are each a population of patients with diabetes having no myocardial infarction clinical history.

13. A method for determining the risk of arteriosclerotic disease according to claim 1, wherein the combination of plural genetic polymorphisms is selected so that,
when cases having a carotid arterial intima-media thickness at least 0.2 mm larger than the average of carotid arterial intima-media thickness of healthy subjects are defined as arteriosclerotic disease cases and the other cases are defined as non-arteriosclerotic disease cases,
cases having a combination of plural genetic polymorphisms showing a significant positive correlation with the carotid arterial intima-media thickness occupy 70% or more of a population of arteriosclerotic disease cases comprising at least 150 cases, and cases having a combination of plural genetic polymorphisms showing a significant positive correlation with the carotid arterial intima-media thickness occupy 35% or less of a population of non-arteriosclerotic disease cases comprising at least 150 cases.

14. A method for determining the risk of arteriosclerotic disease according to claim 1, wherein the combination of plural genetic polymorphisms is a combination of 5 or less of genetic polymorphisms.

15. A method for determining the risk of arteriosclerotic disease according to claim 1, further comprising a risk evaluation process for evaluating the risk of arteriosclerosis caused by an environmental factor from the information of the subject on the environmental factor based on the risk of arteriosclerosis inherent to the environmental factor.

16. A method for determining the risk of arteriosclerotic disease according to claim 1, comprising plural risk evaluation processes and further comprising a process for calculating the risk of arteriosclerotic disease by considering all the risks of arteriosclerosis determined in the individual risk evaluation processes.

17. A method for determining the risk of arteriosclerotic disease according to claim 1, further comprising a detection process for detecting the genotype of the subject on the plural genetic polymorphisms before a risk evaluation process.

18. A method for detecting genetic polymorphisms comprising a process for detecting a genotype of a subject on genetic polymorphisms constituting at least one set of arteriosclerosis-associated genetic polymorphisms selected from the sets of arteriosclerosis-associated genetic polymorphisms shown in Tables 9-1 and 9-2,
wherein the detection result is used for determining the risk of arteriosclerotic disease.

19. A method for detecting genetic polymorphisms comprising a process for detecting a genotype of a subject on genetic polymorphisms constituting at least one set of arteriosclerosis-associated genetic polymorphisms selected from the sets of arteriosclerosis-associated genetic polymorphisms shown in Tables 1-1, 1-2, 1-3 and 1-4,
wherein the detection result is used for determining the risk of arteriosclerotic disease.

20. A genetic marker comprising genetic polymorphisms constituting at least one set of arteriosclerosis-associated genetic polymorphisms selected from
the sets of arteriosclerosis-associated genetic polymorphisms shown in Tables 9-1 and 9-2.

21. A genetic marker comprising genetic polymorphisms constituting at least one set of arteriosclerosis-associated genetic polymorphisms selected from
the sets of arteriosclerosis-associated genetic polymorphisms shown in Tables 1-1, 1-2, 1-3 and 1-4.

22. A kit for analyzing genetic polymorphisms comprising a pair of primers capable of specifically amplifying genes constituting at least one set of arteriosclerosis-associated genetic polymorphisms selected from the sets of arteriosclerosis-associated genetic polymorphisms shown in Tables 9-1 and 9-2 or a nucleic acid probe capable of specifically hybridizing the genes,
wherein the kit is so configured as to detect at least one of the sets of arteriosclerosis-associated genetic polymorphisms shown in Tables 9-1 and 9-2.

23. A kit for anlalyzing genetic polymorphisms according to claim 22, wherein the sets of arteriosclerosis-associated genetic polymorphisms comprises at least 50% of the genetic polymorphisms shown in Tables 10-1 and 10-2.

24. A kit for anlalyzing genetic polymorphisms according to claim 22, wherein the sets of arteriosclerosis-associated genetic polymorphisms comprises at least 90% of the genetic polymorphisms shown in Tables 10-1 and 10-2.

25. A kit for analyzing genetic polymorphisms according to claim 22, comprising a primer or a probe for detecting at least two genetic polymorphisms selected from the genetic polymorphisms shown in Tables 9-1 and 9-2,
when cases having a carotid arterial intima-media thickness at least 0.2 mm larger than the average of carotid arterial intima-media thickness of healthy subjects are defined as arteriosclerotic disease cases and the other cases are defined as non-arteriosclerotic disease cases,
cases having a combination of genotypes having a significant positive correlation with the carotid arterial intima-media thickness occupy 70% or more of a population of arteriosclerotic disease cases comprising at least 150 cases, and
occupy 35% or less of a population of non-arteriosclerotic disease cases comprising at least 150 cases, the combination of genotypes being in sets of arteriosclerosis-associated genetic polymorphisms shown in Tables 1-1 to 1-4 which are capable of including the selected genotypes.

26. A kit for analyzing genetic polymorphisms comprising a pair of primers capable of specifically amplifying genes constituting at least one set of arteriosclerosis-associated genetic polymorphisms selected from the sets of arteriosclerosis-associated genetic polymorphisms shown in Tables 1-1, 1-2,1-3 and 1-4 or a nucleic acid probe capable of specifically hybridizing the genes,
wherein the kit is so configured as to detect at least one of the sets of arteriosclerosis-associated genetic polymorphisms shown in Tables 1-1, 1-2, 1-3 and 1-4.

27. A kit for anlalyzing genetic polymorphisms according to claim 26, wherein the sets of arteriosclerosis-associated genetic polymorphisms comprises at least 50% of the genetic polymorphisms shown in Tables 2-1 and 2-2.

28. A kit for anlalyzing genetic polymorphisms according to claim 26, wherein the sets of arteriosclerosis-associated genetic polymorphisms comprises at least 90% of the genetic polymorphisms shown in Tables 2-1 and 2-2.

29. A kit for analyzing genetic polymorphisms according to claim 26, comprising at least any of primers and probes for detecting at least two genetic polymorphisms selected from the genetic polymorphisms shown in Tables 2-1 and 2-2,
wherein, when cases having a carotid arterial intima-media thickness at least 0.2 mm larger than the average of carotid arterial intima-media thickness of healthy subjects are defined as arteriosclerotic disease cases and the other cases are defined as non-arteriosclerotic disease cases,
cases having at least one combination of genotypes having a significant positive correlation with the carotid arterial intima-media thickness occupy 30% or more of a population of arteriosclerotic disease cases comprising at least 150 cases, and occupy 15% or less of a population of non-arteriosclerotic disease cases comprising at least 150 cases, the combination of genotypes being in sets of arteriosclerosis-associated genetic polymorphisms shown in Tables 1-1, 1-2,1-3 and 1-4 which are capable of including the selected genotypes.

30. An array for determining the risk of arteriosclerotic disease comprising a probe for detecting genetic polymorphisms constituting at least one set of arteriosclerosis-associated genetic polymorphisms selected from the sets of arteriosclerosis-associated genetic polymorphisms shown in Tables 9-1 and 9-2.

31. An array for determining the risk of arteriosclerotic disease comprising a probe for detecting genetic polymorphisms constituting at least one set of arteriosclerosis-associated genetic polymorphisms selected from the sets of arteriosclerosis-associated genetic polymorphisms shown in Tables 1-1, 1-2,1-3 and 1-4.

32. An apparatus for determining the risk of arteriosclerotic disease using a computer, comprising:
a data table on the risk of arteriosclerosis in which combinations of plural genetic polymorphisms are listed with corresponding risks of arteriosclerosis, and
detection means for checking inputted combinations of plural genetic polymorphisms of a subject against the combinations of plural genetic polymorphisms in the data table on the risk of arteriosclerosis and, when there is a combination of genetic polymorphisms matching between the two, detecting the risk of arteriosclerosis corresponding to the combination of genetic polymorphisms.

33. An apparatus for determining the risk of arteriosclerotic disease according to claim 32, wherein 1 unit is coordinated as the risk of arteriosclerosis with a combination of plural genetic polymorphisms having a significant positive correlation with the carotid arterial intima-media thickness in the data table on the risk of arteriosclerosis in which the combinations of plural genetic polymorphisms are listed with corresponding risks of arteriosclerosis.

34. An apparatus for determining the risk of arteriosclerotic disease according to claim 32, wherein an odds ratio for the carotid arterial intima-media thickness of exceeding a normal range is coordinated as the risk of arteriosclerosis with a combination of plural genetic polymorphisms having a significant positive correlation with the carotid arterial intima-media thickness in the data table on the risk of arteriosclerosis in which the combination of plural genetic polymorphisms are listed with corresponding risks of arteriosclerosis.

35. An apparatus for determining the risk of arteriosclerotic disease according to claim 32, wherein an increase in carotid arterial intima-media thickness is coordinated as the risk of arteriosclerosis with a combination of plural genetic polymorphisms having a significant positive correlation with the carotid arterial intima-media thickness in the data table on the risk of arteriosclerosis in which the combination of plural genetic polymorphisms are listed with corresponding risks of arteriosclerosis.

36. An apparatus for determining the risk of arteriosclerotic disease according to claim 32, further comprising:
a data table on the risk of arteriosclerosis in which the presence or absence of, or the numerical value of an environmental factor is listed with a corresponding risk of arteriosclerosis; and
detection means for checking an inputted presence or absence of, or the numerical value of an environmental factor of a subject against the presence or absence of, or the numerical value of an environmental factor in the data table on the risk of arteriosclerosis, and detecting a risk of arteriosclerosis corresponding to the presence or absence of, or the numerical value of the environmental factor.

37. A recording medium readable by computer, comprising a program for determining the risk of arteriosclerotic disease recorded thereon,
wherein the program comprises the steps of:
checking inputted combinations of plural genetic polymorphisms of a subject against the combinations of plural genetic polymorphisms in a data table on the risk of arteriosclerosis in which combinations of plural genetic polymorphisms are listed with corresponding risks of arteriosclerosis, the data table being recorded in a computer; and
detecting the risk of arteriosclerosis corresponding to the combination of genetic polymorphisms when there is a combination of genetic polymorphisms matching between the two as a result of checking.


SEQUENCE LISTING (8 sequences)

SEQ ID NO: 1 - DNA, 90 bp, Homo sapiens
acagaccacagacctggtccccaaaagaaatggaggcaataggttttgaggggcatgrggacggggttcagcctccagggtvvtacacac

SEQ ID NO: 2 - DNA, 55 bp, Homo sapiens
cgcggacatggaggacgtgygcggccgcctggtgcagtaccgcggcgaggtgcag

SEQ ID NO: 3 - DNA, 75 bp, Homo sapiens
gtgggaggcaacctgctggtcatcgtggccatcgctyggacgccaagactgcagaccatgaccaacgtgttcgtg

SEQ ID NO: 4 - DNA, 66 bp, Homo sapiens
gaaatgtgaacatgtgggactttgtgctstcaccagatgagattaacaccatctatcttggcgggc

SEQ ID NO: 5 - DNA, 73 bp, Homo sapiens
gaccctgcccccagggctcctgaygcccacacccaagctggagaagctcagtctggctaacaacaacttgact

SEQ ID NO: 6 - DNA, 54 bp, Homo sapiens
gggagctctggacctggaagtrctggaagctggaactctgggagctctggaact

SEQ ID NO: 7 - DNA, 61 bp, Homo sapiens
ctttaacattattttgaagtgattggtkgtggtatgaattaacagtttaaatttaaatcct

SEQ ID NO: 8 - DNA, 70 bp, Homo sapiens
ggcggccgtgggcggagccgcgacctgaacgcygggagacctcaccaggcggaggcggtgtctacctcac